(19) United States
(12) Patent Application Publication
(10) Pub. No.: US 2026/0204382 A1
(43) Pub. Date: Jul. 16, 2026

(54) EVALUATING MORPHOLOGY AND OPERATION OF A JOINT

(71) Applicant: Children's Medical Center Corporation, Boston, MA (US)

(72) Inventors: Ata Kiapour, Wayland, MA (US); Mohammadreza Movahhedi, Natick, MA (US); Nazgol Tavabi, Boston, MA (US); Eduardo N. Novais, Boston, MA (US); Young-Jo C. Kim, Boston, MA (US)

(73) Assignee: Children's Medical Center Corporation, Boston, MA (US)

(21) Appl. No.: 19/134,203

(22) PCT Filed: Nov. 30, 2023
(86) PCT No.: PCT/US2023/081781

§ 371 Date: May 29, 2025

Related U.S. Application Data
(60) Provisional application No. 63/428,895, filed on Nov. 30, 2022.

(51) Int. Cl. G16H 20/30; A61B 6/50; A61B 8/08; G06T 7/00; G06T 7/11; G06T 7/246; G06T 7/73; G06T 11/26; G06T 13/40; G06T 17/00

(52) U.S. Cl. CPC G16H 20/30; A61B 6/505; A61B 8/0875; G06T 7/0012; G06T 7/11; G06T 7/251; G06T 7/75; G06T 11/26; G06T 13/40; G06T 17/00; G06T 2200/04; G06T 2207/20036; G06T 2207/30008; G06T 2210/41

(57) ABSTRACT

Described herein are example techniques for evaluating morphology and/or operation of a joint of an animal. In embodiments, a computer model of the joint is prepared and analyzed. An amount of translation movement and an amount of rotation movement to be simulated are determined and used in simulated movement of the bone(s) of the joint across many different positions. The positions that are simulated with the computer model may correspond to different positions of the bone during operation of the joint, such as (in the case of a human hip joint) positions a bone may take during walking, running, or other movements. These positions may be automatically simulated based on the amounts of translation and rotation. In some embodiments, determined information regarding the morphology and/or operation of the joint from the simulation may be used to determine a recommendation of whether and/or how to treat improper operation of the joint.

### CROSS REFERENCE TO RELATED APPLICATION

This application claims the benefit under 35 U.S.C. § 119 (e) of U.S. Provisional Patent Application No. 63/428,895 filed Nov. 30, 2022, the contents of which application are incorporated herein by reference in their entirety.

### BACKGROUND

The hip joint (scientifically referred to as the femoroacetabular joint) is the joint between the head of the femur and the acetabulum of the pelvis. The hip joint supports the weight of the body in both static (e.g., standing) and dynamic (e.g., walking/running, for example) postures. The hip joint plays roles in retaining balance and in maintaining the pelvic inclination angle.

The hip joint may succumb to many disorders, which may result from and/or be associated with nervous issues, osteoarthritic issues, infections, traumatic events, and/or genetic issues. Patients may receive clinical care for hip disorders that limit range of motion, which can include, but are not limited to, femoroacetabular impingement (FAI), hip instability (e.g., hip dysplasia), and the like. Many adolescent and adult patients with FAI or hip instability are considered to be at a high risk of hip osteoarthritis (OA) and/or an early need for total hip replacement.

### SUMMARY

In one embodiment, there is provided a method comprising evaluating morphology and/or operation of a joint of an animal using a computer model of the joint. The joint comprises a first bone and a second bone that move relative to one another during use of the joint by the animal. The computer model of the joint comprises a first model of the first bone and a second model of the second bone. The evaluating the morphology and/or operation of the joint comprises determining the morphology of tissue of the joint, wherein determining the morphology comprises size/dimensions, orientations, relative positions, and/or alignments of one or more anatomical features of the first bone and/or the second bone, determining a first amount of translation movement of the first bone to simulate, and determining a second amount of rotation movement of the first bone to simulate. The evaluating further comprises simulating movement of the first bone of the joint across a plurality of positions with respect to the second bone. The simulating of the first bone across the plurality of positions comprises simulating translation movement of the first bone in accordance with the first amount and simulating rotation movement of the first bone in accordance with the second amount. The evaluating further comprises determining, based on the simulating of the movement of the first bone of the joint, information about operation of the joint. The method further comprises, in response to determining that the morphology of the joint is improper and/or the information about operation of the joint indicates improper operation, determining a recommendation on whether and/or how to treat the joint based on the information about the morphology and/or operation of the joint and outputting the recommendation on whether and/or how to treat the joint. In some embodiments, the evaluating further includes comparing the measured joint/tissue morphology and function with data from demographically matched control subjects to determine personalized injury risk and treatment options.

In another embodiment, there is provided a method comprising generating different 3D and 2D representations of the joints/tissues from imaging data, which can be augmented/annotated with quantitative information on the joint/tissue morphology and function (e.g., impingement and coverage) that can be exported to reports or web-based interfaces for evaluations by the users.

In another embodiment, there is provided at least one computer-readable storage medium having encoded thereon executable instructions that, when executed by at least one processor, cause the at least one processor to carry out a method. The method comprises evaluating morphology and/or operation of a joint of an animal using a computer model of the joint. The joint comprises a first bone and a second bone that move relative to one another during use of the joint by the animal. The computer model of the joint comprises a first model of the first bone and a second model of the second bone. The evaluating the morphology and/or operation of the joint comprises determining the morphology of tissue of the joint, wherein determining the morphology comprises size/dimensions, orientations, relative positions, and/or alignments of one or more anatomical features of the first bone and/or the second bone, determining a first amount of translation movement of the first bone to simulate, and determining a second amount of rotation movement of the first bone to simulate. The evaluating further comprises simulating movement of the first bone of the joint across a plurality of positions with respect to the second bone. The simulating of the first bone across the plurality of positions comprises simulating translation movement of the first bone in accordance with the first amount and simulating rotation movement of the first bone in accordance with the second amount. The evaluating further comprises determining, based on the simulating of the movement of the first bone of the joint, information about operation of the joint.

In a further embodiment, there is provided an apparatus. The apparatus comprises at least one processor and at least one storage medium having encoded thereon executable instructions that, when executed by the at least one processor, cause the at least one processor to carry out a method. The method comprises evaluating morphology and/or operation of a joint of an animal using a computer model of the joint. The joint comprises a first bone and a second bone that move relative to one another during use of the joint by the animal. The computer model of the joint comprises a first model of the first bone and a second model of the second bone. The evaluating the morphology and/or operation of the joint comprises determining the morphology of tissue of the joint, wherein determining the morphology comprises size/dimensions, orientations, relative positions, and/or alignments of one or more anatomical features of the first bone and/or the second bone, determining a first amount of translation movement of the first bone to simulate, and determining a second amount of rotation movement of the first bone to simulate. The evaluating further comprises simulating movement of the first bone of the joint across a plurality of positions with respect to the second bone. The simulating of the first bone across the plurality of positions comprises simulating translation movement of the first bone in accordance with the first amount and simulating rotation movement of the first bone in accordance with the second amount. The evaluating further comprises determining, based on the simulating of the movement of the first bone of the joint, information about operation of the joint. The method further comprises, in response to determining that the morphology of the joint is improper and/or the information about operation of the joint indicates improper operation, determining a recommendation on whether and/or how to treat the joint based on the information about the morphology and/or operation of the joint and outputting the recommendation on whether and/or how to treat the joint.

In another embodiment, there is provided a method comprising generating different 3D and 2D representations of the joints/tissues from imaging data, which can be augmented/annotated with quantitative information on the joint/tissue morphology and function (e.g., impingement and coverage) that can be exported to reports or web-based interfaces for evaluations by the users.

The foregoing is a non-limiting summary of the invention, which is defined by the attached claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a diagram of a system with which some embodiments may operate;

FIG. 2 is a flowchart of a process that may be implemented in some embodiments to analyze a joint;

FIG. 3 illustrates examples of common hip pathologies;

FIG. 4 illustrates examples of common surgical procedures to reduce impingement and improve hip stability;

FIG. 5 is a flowchart of a process that may be performed in some embodiments by a platform operating in accordance with some techniques described herein;

FIG. 6 illustrates an example of a technique for analysis of medical imagery, in accordance with some embodiments;

FIG. 7 illustrates pelvis- and femur-based coordinate systems that may be used in some embodiments;

FIG. 8 specifies femoral head and neck and pelvic acetabulum on the main femur and pelvis models, in accordance with some embodiments;

FIG. 9 illustrates a technique that may be used in some embodiments to determine the femoral head (FH) axis based on the inertia axes of femoral head model;

FIG. 10 illustrates a technique that may be used in some embodiments to determine the femoral neck (FN) axis based on the bottom and top surfaces of the femoral neck model;

FIG. 11 illustrates a technique that may be used in some embodiments to determine the shaft axis;

FIG. 12 illustrates a technique that may be used in some embodiments to determine the posterior condyle axis;

FIG. 13 illustrates a technique that may be used in some embodiments for specifying acetabular labrum and face-clocks;

FIG. 14 illustrates a technique that may be used in some embodiments to determine femoral head quadrants and clock-face;

FIG. 15 illustrates a technique that may be used in some embodiments to find the sacrum top plane;

FIG. 16 illustrates a technique that may be used in some embodiments for femoral version measurement;

FIG. 17 illustrates a technique that may be used in some embodiments for femoral mechanical-anatomical angle and femoral offset measurement;

FIG. 18 illustrates a technique that may be used in some embodiments for femoral neck-shaft angle measurement;

FIG. 19 illustrates a technique that may be used in some embodiments for femoral head-neck tilt angle measurement in coronal, sagittal, and axial views;

FIG. 20 illustrates a technique that may be used in some embodiments for femoral head-neck offset and Alpha angle measurement;

FIG. 21 illustrates a technique that may be used in some embodiments for CE angle measurement;

FIG. 22 illustrates a technique that may be used in some embodiments for acetabular version measurement;

FIG. 23 illustrates a technique that may be used in some embodiments for acetabular angle (AA) and acetabular index (AI) measurement;

FIG. 24 illustrates a technique that may be used in some embodiments for migration percentage measurement;

FIG. 25 illustrates a technique that may be used in some embodiments for femoral epiphyseal morphology and dimensions;

FIG. 26 illustrates a technique that may be used in some embodiments for acetabular surface thickness map and acetabulum dimensions;

FIG. 27 illustrates a technique that may be used in some embodiments to show 3D acetabular deficiency compared to demographically matched controls;

FIG. 28 illustrates a technique that may be used in some embodiments for sacro-Pelvic parameters measurement;

FIG. 29 illustrates a technique that may be used in some embodiments for joint space width measurements;

FIG. 30 illustrates comparisons between an experienced human examiner (manual) and the automated methods disclosed herein in measuring key morphological features of the hip joint;

FIG. 31 illustrates a technique that may be used in some embodiments to show 3D femoral head coverage compared to demographically matched controls;

FIG. 32 indicates associations between changes in hip center of rotation during hip flexion-extension as well as increased hip translation range and impingement free hip flexion-extension, as determined in accordance with some embodiments;

FIG. 33 indicates associations between changes in hip center of rotation during hip abduction-adduction as well as increased hip translation range and impingement free hip abduction-adduction, as determined in accordance with some embodiments;

FIG. 34 indicates associations between changes in hip center of rotation during hip axial rotation as well as increased hip translation range and impingement free hip axial rotation, as determined in accordance with some embodiments;

FIG. 35 indicates associations between changes in hip center of rotation during hip abduction-adduction at 90 degrees of flexion as well as increased hip translation range and impingement free hip abduction-adduction 90 degrees of flexion, as determined in accordance with some embodiments;

FIG. 36 indicates associations between changes in hip center of rotation during hip axial rotation at 90 degrees of flexion as well as increased hip translation range and impingement free hip axial rotation at 90 degrees of flexion, as determined in accordance with some embodiments;

FIG. 37 indicates associations between changes in hip center of rotation during hip external rotation at 30 degrees of extension as well as increased hip translation range and impingement free hip axial rotation at 90 degrees of flexion, as determined in accordance with some embodiments;

FIG. 38 indicates associations between changes in hip center of rotation during hip external rotation at 60 degrees of flexion+50 degrees of abduction as well as increased hip translation range and impingement free hip external rotation at 60 degrees of flexion+50 degrees of abduction, as determined in accordance with some embodiments;

FIG. 39 indicates associations between changes in hip center of rotation during hip internal rotation at 90 degrees of flexion+30 degrees of abduction as well as increased hip translation range and impingement free hip internal rotation at 90 degrees of flexion+30 degrees of abduction, as determined in accordance with some embodiments;

FIG. 40 indicates associations between increased hip translation range and impingement free range of motion, as determined in accordance with some embodiments;

FIG. 41 illustrates density plots indicating the impingement free range of motion across a tested range of motion, in accordance with some embodiments;

FIG. 42 illustrates impingement risk assessments per mode of motion which may help with identifying the motions (activities) leading to more impingement, in accordance with some embodiments;

FIGS. 43 and 44 illustrate positional impingement maps which highlight regions of impingement across femur and pelvis for a single simulated position, in accordance with some embodiments;

FIG. 45 illustrates global impingement maps highlighting the regions of impingement within the tested range of motion, in accordance with some embodiments;

FIG. 46 illustrates changes in femoral head coverage as percentage of overall area by increased hip translation during the neutral position compared to demographically matched controls in accordance with some embodiments;

FIG. 47 illustrates changes in femoral head coverage as percentage of overall area by increased hip translation during hip flexion compared to demographically matched controls (green is normal range, yellow is borderline abnormal range, and pink is abnormal range), in accordance with some embodiments;

FIG. 48 illustrates changes in femoral head coverage as percentage of overall area by increased hip translation during hip extension compared to demographically matched controls (green is normal range, yellow is borderline abnormal range, and pink is abnormal range), in accordance with some embodiments;

FIG. 49 illustrates changes in femoral head coverage as percentage of overall area by increased hip translation during hip external rotation compared to demographically matched controls (green is normal range, yellow is borderline abnormal range, and pink is abnormal range), in accordance with some embodiments;

FIG. 50 illustrates changes in femoral head coverage as percentage of overall area by increased hip translation during hip external rotation and extension compared to demographically matched controls (green is normal range, yellow is borderline abnormal range, and pink is abnormal range), in accordance with some embodiments;

FIG. 51 illustrates changes in femoral head coverage as percentage of overall area by increased hip translation during hip external rotation+extension and abduction compared to demographically matched controls (green is normal range, yellow is borderline abnormal range and pink is abnormal range), in accordance with some embodiments;

FIG. 52 illustrates a 3D representation of the regional femoral head coverage during a specific motion, in accordance with some embodiments;

FIGS. 53-62 illustrate examples of user interfaces for some implementations of a joint analysis platform, with which some embodiments may operate;

FIGS. 63 and 64 illustrate examples of reconstructed 3D models and associated measurements of joint morphology and function exported to electronic medical records (e.g., PACS radiology database) user interfaces for some implementations of a joint analysis platform, with which some embodiments may operate;

FIG. 65 illustrates an example of development of 3D models from MRI and definition of anatomical coordinate system, in accordance with some embodiments;

FIG. 66 illustrates an example of model registration to calculate the relative rotation and translation of the femur with respect to the neutral position, in accordance with some embodiments; and

FIG. 67 is a block diagram of a computing device with which some embodiments may operate.

### DETAILED DESCRIPTION

Described herein are examples of techniques for evaluating detailed morphology of a joint, including its associated tissue structures, and evaluating operation of a joint, such as the hip joint. In some embodiments described herein, a 3D computer model of the joint is prepared and analyzed, which includes a model of a first bone of a joint and a second bone of the joint. The 3D models may be automatically oriented into an anatomical coordinate system based on anatomical features of the joint. The aligned 3D bone models may be used to automatically measure detailed morphology of each bone and the joint, in 3D. An amount of translation movement and an amount of rotation movement to be simulated for the first bone of the joint are determined and used in simulated movement of the first bone of the joint across many different positions. The positions that are simulated with the computer model may correspond to different positions of the bone during operation of the joint, such as positions a bone may take during walking, running, or other movements. These positions may be automatically simulated based on the amounts of translation and rotation. The measured morphology and/or simulated hip movement may be used to determine information about operation of the joint, which may include information on whether the joint is pathologic (e.g., abnormal morphology) or operating in a pathologic manner. Such information on the joint may include information on whether movement of bones within the joint exhibits pathologic operation, such as whether the bones impinge during operation of the joint or whether the bones show instability (e.g., risk of dislocation) during operation. Such analyses may include information on locations and/or degree of impingement and joint coverage. Such analyses may also include information on degrees of morphology correction (e.g., bone resection or bony fragment realignment), and motion correction (e.g., rotation and/or translation) that would reduce or eliminate pathologic operation of the joint. In some embodiments, the determined information regarding the morphology and/or operation of the joint may be the output. In some embodiments, the determined information regarding the measured joint/tissue morphology and function may be compared with data from demographically matched control subjects to determine personalized injury risk and treatment options. In other embodiments, the determined information regarding the morphology and/or operation of the joint may additionally or alternatively include determining whether the information about the joint indicates improper (e.g., pathologic) morphology and/or operation and, in response to such a determination, determining and outputting a recommendation of whether and/or how to treat the joint. The determination of the recommendation may be based on the information about the joint. In some such embodiments, the determination may include whether to recommend surgery or physical therapy for the joint, based on the determination information about the joint (e.g., morphology, impingement, joint coverage, amount of rotation/translation needed to reduce or eliminate improper operation, or other information). In another embodiment, there is provided a method comprising generating different 3D and 2D representations of the joints/tissues from imaging data. The representations can be augmented/annotated with quantitative information on the joint/tissue morphology and function (e.g., impingement and coverage) that can be exported to reports or web-based interfaces for evaluations by the users (e.g., patients and clinicians).

Hip preservation surgery has for decades been the primary treatment for improving or restoring to normal hip joint function, minimizing risk of hip OA, and forestalling hip replacement (e.g., avoiding or delaying insertion of an artificial hip). Hip preservation may include arthroscopy or other surgical procedures in which tissues of the hip are modified, such as by modifying connective tissues, cartilage, bone, or other tissues. This may include modifying the shape of bone in the hip joint to avoid improper contact between the femoral head and the acetabulum, which may be a source of pain. Modifying the bone may include removing or reducing the size of conformational abnormalities in bone.

Success of hip preservation surgery is generally surgeon dependent, with better outcomes being directly tied to the skill of the surgeon and worse outcomes resulting from lower-skill surgeons. But the inventor has recognized and appreciated that even the best outcomes are still limited in success or worse, may even be unnecessary.

Planning for such conventional hip surgeries relies on clinical examinations and usually a two-dimensional (2D) assessment of morphological abnormalities, for identifying a source of difficulty (e.g., a source of pain in a hip joint). Conventional morphological assessments of the hip joint involve 2D measurements done on plain x-rays or even 3D imaging modalities such as CT scan or MRI. Such approaches fail to capture 3D abnormalities of the hip joint and bones.

Conventional modeling techniques may be used to estimate how a head of a femur may rotate within the acetabulum. Such conventional modeling relies on an assumption that a hip joint is a ball-and-socket joint and that the ball of the femoral head merely rotates within the socket of the acetabulum. Such models may be manually manipulated by a user using a computer user interface, with the user manually instructing each specific move of (e.g., clicking and dragging) the femur to simulate a patient moving, such as walking. During the manual simulated movement, the femur model rotates with respect to the pelvis model with the femoral head articulating inside the acetabulum. The user may then attempt to identify, through inspection during the manual manipulation of the simulation, whether and where the femur contacts the pelvis during rotation, or otherwise evaluate whether and how the rotation of the femur differs from a that of a conformationally normal hip joint. Based on an assumption that identified areas of contact between bones during rotation are the underlying cause of the hip difficulty/pain, or the lack of femoral head coverage by the acetabulum during rotation is the underlying cause of hip instability, surgical planning is done to eliminate that contact or adjust that coverage and surgery is performed according to the plan, in which hip joint tissues are modified.

The inventor has recognized and appreciated that despite that this type of surgical planning has been done for decades by the best orthopedic surgeons, and this type of manual manipulation of a computer model of a joint to rotate the femur has been done for surgical planning for many years, it is fundamentally flawed. The computer model rests on an inaccurate assumption. The medical system has for decades or longer believed that the femoral head merely rotates in place in the hip joint, replicating a perfect ball and socket joint. Innumerable surgeries on innumerable patients have been conducted under this assumption. But it is simply incorrect. The inventor has recognized and appreciated that even in normal, asymptomatic individuals, the femoral head does not solely rotate within the acetabulum, but also translates (glides) in addition to rotating. Such translation may include moving in one or more directions or along one or more axes.

Modeling in which a femoral head solely rotates will provide different information on points of contact and amount of coverage between bones than a model in which the femoral head translates as well as rotates. The translation will result in distances and coverages between bones changing and bones may not contact or covered in some areas, and/or contact or be covered in others, than would be predicted by a model in which the femoral head merely rotates. Because a model that solely includes rotation is inaccurate, surgical decisions guided by a model that solely includes rotation are also at risk of being inaccurate. Surgeries based on such models may remove tissues (including bone) that should not be removed or adjust tissues (including bones) that should not be adjusted or leave in place tissues (including bones) that should be removed or adjusted. As such, conventional modeling has been and still is less reliable or unreliable, and surgeries planned with such modeling have been and are misguided or incorrect.

Described herein are techniques for automatic assessment of joint (e.g., hip or shoulder) morphology and techniques for modeling joints (e.g., hip or shoulder), in which the bones of the model may translate in addition to rotate. By comprehensively assessing the morphology in 3D, a more accurate view of morphological abnormality may be generated, which may help in a more accurate diagnosis of the pathology and a better view of abnormality location and pattern, which are often missed in common 2D measurement techniques. The automatic 3D assessment also enables a much faster and reproducible measurement, which are key considerations in clinical operations and care. By modeling movement of a bone relative to one another in a joint along one or more axes, in addition to rotating relative to the other and around one or more axes, a more accurate and physiologically relevant view of movements of bones in a joint may be obtained. When a more accurate/physiologic view of movement of bones in a joint may be obtained, a more accurate/physiologic view of sources of difficulty in a joint may be obtained, such as a determination of whether and where there is impingement or under/over coverage in the joint during movement of the bones.

In some embodiments, in addition to modeling movement of a bone in a joint relative to another in the joint in the direction of one or more axes in addition to rotating, techniques described herein may enable a simulation of range of motion within the joint. In some such embodiments, a user (e.g., a clinician, such as a surgeon) may provide as input to a model information regarding an amount of translation and an amount of rotation to be simulated. The amounts that are input may be associated with a bone of the joint, such as an amount of translation and rotation of a bone of the joint. In the case of a hip joint, this may be or include an amount of translation and rotation of the femoral head within the hip joint. The amounts may be expressed in different manners, including as ranges of motion to be simulated, which in some embodiments may be expressed in units of distance for a limb to traverse and/or degrees of rotation of a limb relative to the joint. These received amounts may be used to simulate movement of the bone in the joint, such as by identifying a number of different positions (in accordance with the amounts of translation and rotation) into which to move a bone of the joint in the simulation across the received amounts. In some cases, this may include more than one billion simulated positions. These positions may be identified by the system and then the model automatically manipulated to place the bones into these positions, and the model may be analyzed to determine information on operation of the joint across the positions. For example, the model may be analyzed to determine whether the joint exhibits pathologic operation in the positions. This may include whether the bones of the joint impinge on one another across the positions or the femoral head is under/over covered by the acetabulum across the positions.

In some embodiments, generation of a computer model of a joint may be guided by information from a clinical examination of a joint of a patient. The information from the clinical examination of the joint may, in some cases, include information resulting from a physical exam and/or medical imaging of the patient. For example, the amount(s) that are input for translation and/or rotation may be, in some embodiments, based on a clinical examination of a patient. This may include a physical exam in which a patient moves a joint or a clinician moves a joint of a patient to determine range of motion of the patient. This may additionally or alternatively include imaging of a joint, such as imaging that may reveal structure and/or function of the joint, including amounts of translation and/or rotation. For example, in some embodiments, a clinical exam of a patient may include medical imaging that may reveal information on a structure of the joint, such as x-ray, magnetic resonance imaging (MRI), computed tomography (CT), or other imaging of the joint. This may also include medical imaging that may reveal information on a function of the joint, such as information on operation of the joint that may result from imaging of the joint during movement of the joint of the patient. For example, ultrasound imaging of a patient during movement of the joint may reveal information on translation of a bone in the joint during movement, such as translation of a femoral head during walking. Any suitable medical imaging may be done, as embodiments are not limited in this respect. In embodiments in which a clinical exam reveals information on translation and/or rotation of a joint of a patient, that clinical exam may guide the amounts determined or obtained by a simulator for translation and/or rotation of bones in a joint. For example, when a physical exam or medical imaging reveals a range of motion a patient is able to achieve with a joint, that range of motion may inform the amount(s) of rotation and/or translation to be simulated, such that the simulation corresponds to the joint of the patient.

In some such embodiments, medical imaging may reveal information on anatomical features of the joint, such as locations or dimensions of anatomical features. In some cases, a user (e.g., a clinician or patient) may input the information on the anatomical features to the model, such as by inputting information on locations or dimensions of anatomical features. In other cases, medical imaging may yield medical imagery, such as one or more medical images. In some embodiments, the medical imagery may be analyzed to extract information on anatomical features of the joint, such as by identifying locations and/or dimensions of anatomical features of the joint. Anatomical features may include known landmarks on bones. In the case of a hip joint, such landmarks may include femoral head, femoral neck, acetabular surface, femoral epicondyles, or other landmarks. The locations or dimensions may include positions (e.g., relative positions, such as distances between landmarks), shapes, and/or sizes of these landmarks. In some embodiments, medical imagery of the joint may be analyzed to identify different portions of anatomy depicted therein. For example, bones of a joint (e.g., for a hip joint, the femur or femoral head and the pelvis or acetabulum) may be identified, and/or other tissues in the joint may be identified. Analyzing the medical imagery to identify tissues depicted therein may include segmenting the medical imagery, which may result in an annotated medical image in which content of the image is identified as depicting different anatomy. For example, portions of the joint corresponding to one bone of the joint (e.g., femur) and to another bone of the joint (e.g., part of a pelvis) may be identified as corresponding to such bones. Other tissues such as cartilage or connective tissues may also be identified in segmentation. In some such cases, upon segmentation, locations and/or dimensions of anatomical features may be identified for the different tissues. Based on the information on anatomical features of the joint—such as the segmented medical imagery and/or the locations and/or dimensions of anatomical features—the computer-generated model may be customized to reflect anatomy of the patient. Simulation of movement of the joint may then correspond to movement of the joint of the patient.

In some embodiments, a computer model of a joint and simulated movement of the joint using the computer model of the joint may be used to guide treatment or care of a patient. For example, some pathologic joints may be candidates for surgery or for physical therapy. Conventionally, systems were unable to determine based on simulation whether a joint was a candidate for physical therapy, meaning many patients may have been unnecessarily subjected to surgery. In some embodiments, information on operation of a joint that results from simulation may be used to determine a recommendation for whether and/or how to treat a joint. This may be the case if analysis of the joint indicates improper operation, such as pathologic operation of the joint. For example, impingement of bones and/or under/over coverage of the joint during operation of the joint, or impingement and/or under/over coverage of the joint that satisfies one or more criteria (e.g., more than a threshold amount of impingement and/or under/over coverage) may indicate improper operation of the joint. In such a case, information on operation of the joint may be compared to one or more criteria for physical therapy. As an example of such a criterion, simulation of operation of the joint with the computer model may in some embodiments and for some joints reveal information such as whether there is an amount of translation that, if achieved by the patient, may reduce or eliminate improper operation of the joint. In some such cases, the amount of translation may be compared to a physical therapy criterion, such as by comparing a needed amount of translation to an amount of translation that can be reliably achieved with physical therapy. If the physical therapy criterion is met, physical therapy may be recommended. In some embodiments, when surgery is to be recommended, a recommendation for how to perform the surgery may be output. For example, a recommendation of tissues (e.g., bone) to be removed or adjusted to resolve impingement and/or under/over coverage of the joint may be output. This may include identifying portions of tissue (e.g., bone) that impinge and/or under/over covers and that could be removed to reduce or eliminate impingement and/or under/over coverage of the joint.

Accordingly, described herein are various techniques for evaluating operation of a joint of an animal, such as a hip joint of a human patient. Illustrative embodiments are described below in connection with the figures. It should be appreciated that embodiments are not limited to operating in accordance with the examples below, as other embodiments are possible. In addition, while for ease of readability examples provided below focus on the human hip joint, it should be appreciated that embodiments are not limited to operating with this joint. Embodiments may operate with any other type of joint, socket and/or integration of bones within an animal body (e.g., a human animal or non-human animal, including a human mammal or a non-human mammal) can be analyzed for purposes of performing the disclosed assessment without departing from the scope of the instant disclosure. Techniques described herein may be particularly advantageous when used with ball-and-socket joints such as the human hip and shoulder. Those skilled in the art should also appreciate that techniques used herein, when used with humans, may be used with humans at a variety of ages, sexes, or developmental conditions, including adults or children.

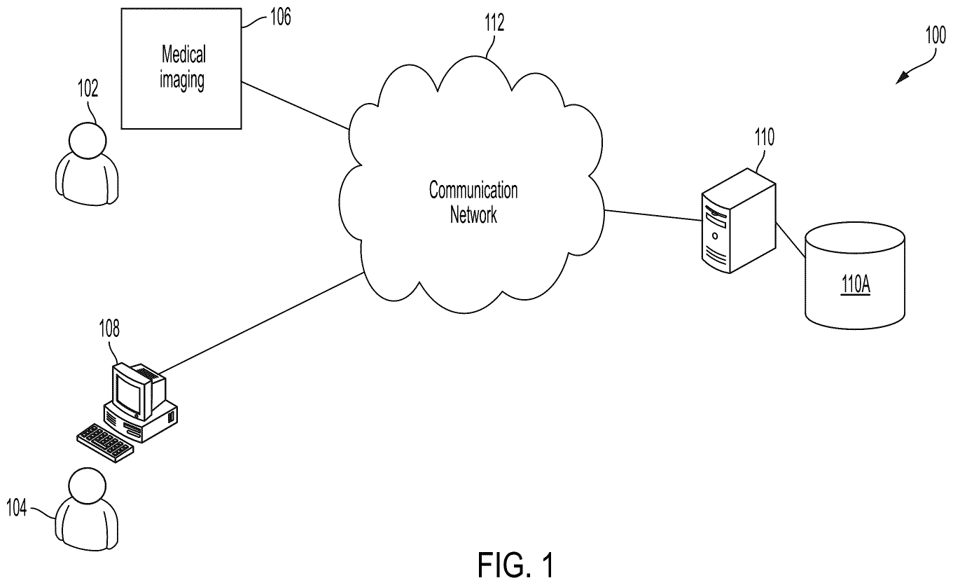

FIG. 1 illustrates a system 100 with which some embodiments may operate. System 100 may be used to evaluate operation of a joint of a patient 102, such as to evaluate operation of a hip joint of the patient 102. A clinician 104 (e.g., physician such as a surgeon or other doctor, a nurse, a nurse practitioner, physician's assistant, surgery tech, or other clinician) may use the system 100 to perform the evaluation and determine whether and how to treat the joint. The system 100 may include medical imaging equipment 106 to perform medical imaging of the patient 102, including of the joint of the patient 102. The medical imaging equipment 106 may be or include any suitable medical imaging equipment, including an MRI scanner, x-ray scanner, CT scanner, ultrasound scanner, or other medical imaging equipment. Embodiments are not limited to use of any particular form of medical imaging.

In some embodiments, a clinician 104 may obtain information on anatomical features of the joint of the patient 102, through physical examination and/or through use of the medical imaging equipment 106. The clinician 104 may input that information on anatomical features, including medical imagery and/or locations and dimensions of anatomical features of the joint, to a client interface of a joint analysis facility. The client interface of the joint analysis facility may execute on the computing device 108 and be operated by the clinician 104 or a colleague of the clinician 104. While illustrated in FIG. 1 as a personal computer, computing device 108 may be any suitable computing device including a desktop or laptop personal computer, a smartphone, a tablet computer, or other device. The client interface may be implemented as a web interface operable through a web browser, as an application program executing on device 108, or in another matter, as embodiments are not limited in this respect.

In accordance with techniques described herein, the clinician 104 may also operate the client interface of the joint analysis facility to provide information on the simulation of the joint to be performed. For example, the clinician 104 may input to the client interface via the device 108 an amount of rotation to be simulated for the joint and an amount of translation to be simulated for the joint. Other input may also be provided, examples of which are provided below.

The joint analysis facility may evaluate joint morphology and/or the operation of the joint of the patient 102 based on the information the facility receives regarding the joint. The joint analysis facility may operate on computing device 110, which may be implemented as one or more servers (rack mounted or otherwise), as an interconnected system of two or more servers sharing processing load such as a cloud computing system, or in any other suitable manner. In some embodiments, the joint analysis facility may receive medical imagery for the joint from the medical imaging equipment 106 and/or from the client interface operating on the computing device 108. In some such embodiments, the joint analysis facility may analyze the medical imagery to determine information on the joint, such as segmenting the medical imagery to determine the tissues (e.g., bones) depicted in the imagery and determine information (e.g., locations and dimensions) on anatomical features depicted in the imagery, such as using computerized object recognition techniques. In other embodiments, the joint analysis facility may additionally or alternatively receive information on anatomical features of the joint that was input by the clinician 104 via the client interface of the computing device 108. As should be appreciated from the foregoing, such information on anatomical features may include information on location and/or dimensions of anatomical features.

In accordance with techniques described herein, the joint analysis facility may generate a computer model of the joint of the patient 102 and analyze the joint using the computer model to determine information on operation of the joint. Such information may indicate whether operation of the joint is pathologic or otherwise improper. The joint analysis facility is not limited to generating the computer model in any particular manner. In some embodiments, the facility may generate a computer model from the medical imagery, such as by segmenting medical imagery and forming computer model objects from the medical imagery and then manipulating those objects. In other embodiments, the facility may have a default model of a joint with default locations and dimensions of anatomical features in the joint, which the facility may then adapt based on the locations and dimensions of anatomical features as determined from analysis of medical imagery and/or from input from the clinician 104. The default model may be a default for all patients. In some embodiments, the joint analysis facility may maintain a data store 110A of information on joints for different demographic groups, such as based on age, sex, gender, race, ethnicity, activity level (e.g., general exercise level or athleticism), or other factors. In some embodiments, a default model may be selected based on received demographic information regarding a patient. In some embodiments, information determined by the joint analysis facility regarding the joint (through the received medical imagery or user input, or from simulation) may be added to the data store 110A to develop further information regarding joints of patients of different demographic groups.

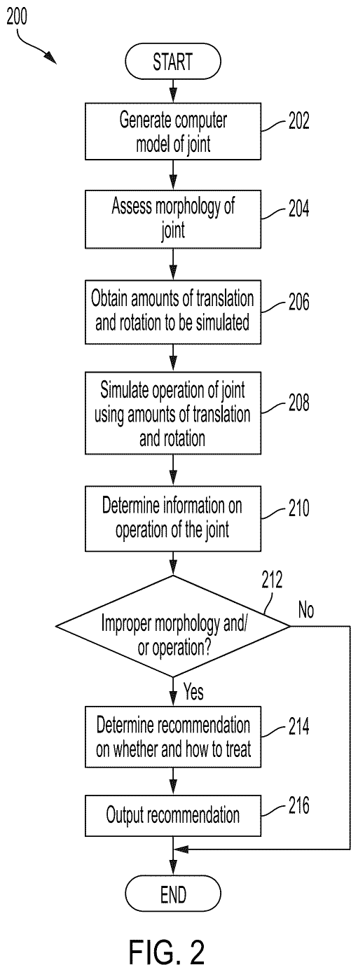

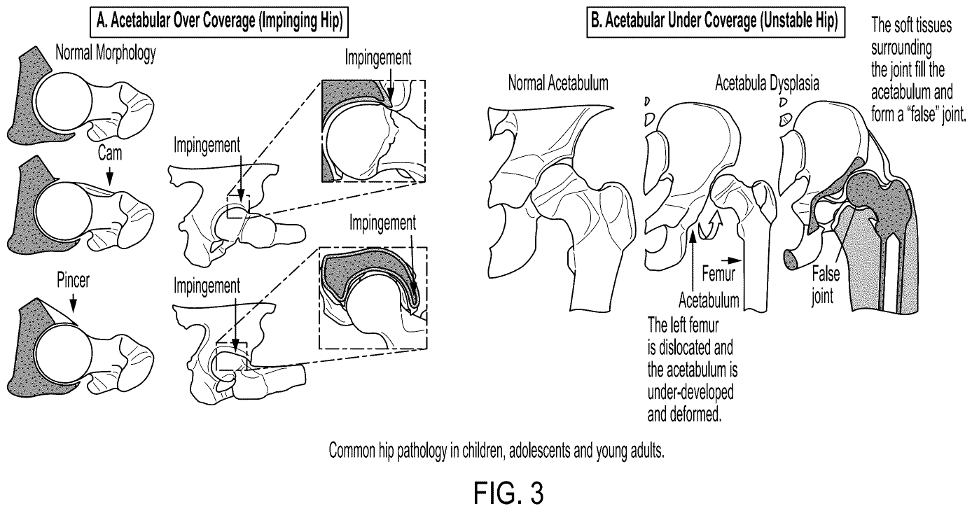

Once the computer model for the joint is developed, simulation may be performed in accordance with techniques described herein. For example, using the amounts of rotation and/or translation input by the clinician 104 via the client interface, the joint analysis facility may simulate movement of the joint, such as by simulating movement of tissues of the joint (e.g., bones) throughout positions defined by the amounts of rotation and translation. This may include automatically moving a bone of the joint through a number of positions defined by the rotation and translation, such as by moving a bone a designated small amount across a large number of iterations until the designated rotation and translation have been achieved. At each position, the joint analysis facility may determine information on operation of the joint, such as information indicating whether the joint is operating in a normal or in a pathologic manner. The facility may determine whether the joint is operating in a pathologic manner by determining whether bones of the joint impinge, determining an amount of coverage of one bone by another in the joint, or determining other information regarding movement of the bones relative to one another. Examples of techniques that a joint analysis facility may implement for this simulation and evaluation of a joint are described below in connection with FIG. 2 as well as in connection with FIGS. 3-68.

The joint analysis facility may generate information regarding operation of the joint through the simulation. Such information may include results of the simulation, which may include determined information on operation of the joint. For example, information on impingement may be generated, which may be a heat map or other representation on locations of the joint at which impingement has been detected through simulation. As another example, information on degree of bone coverage (e.g., femoral head coverage) may be indicated, such as a map indicating locations where there is more or less coverage, or coverage more than one or more thresholds or less than one or more thresholds. As another example, in some embodiments, information regarding operation of the joint may be used by the joint analysis facility to generate a determination of whether and/or how to treat the joint. For example, the facility may determine whether joint operation is pathologic or not (e.g., whether there is impingement and/or under/over coverage) and, accordingly, whether treatment of the joint is recommended or not. In addition, in some embodiments, if the facility determines that treatment is recommended, the facility may determine a treatment to recommend. For example, the facility may determine whether the joint is a candidate for physical therapy or for surgery and provide information regarding the determined treatment recommendation

The joint analysis facility may generate information regarding the morphology of the joint through comprehensive 3D analysis. Such information may include size/dimensions of anatomical features, and the relative position and orientation of anatomical features. For example, information on morphology may be generated, which may be a number (e.g., distance, dimensions, angles and percentages), heat maps or other representation of the relative size, locations and alignments of the joint and its components (e.g., bone and connective tissues). As another example, in some embodiments, information regarding joint morphology may be used by the joint analysis facility to generate a determination of whether and/or how to treat the joint. For example, the facility may determine whether joint operation is pathologic or not (e.g., size/alignment mismatch) and, accordingly, whether treatment of the joint is recommended or not. In addition, in some embodiments, the joint analysis facility may compare the morphological measurements to historic data for other patients that share one or more demographic characteristics (e.g., age, sex, gender, race, ethnicity, occupation, exercise level, etc.) with the patient to determine relative risk of pathology, and, accordingly, whether treatment of the joint is recommended or not. In addition, in some embodiments, if the facility determines that treatment is recommended, the facility may determine a treatment to recommend. For example, the facility may determine whether the joint is a candidate for physical therapy or for surgery and provide information regarding the determined treatment recommendation.

The information that is generated by the facility regarding the joint operation may be output by the joint analysis facility for presentation to the clinician 104 via the client interface of the device 108. Once the information is presented, the clinician 104 may interact with the information, such as interacting with the computer model of the joint, viewing information about operation of the joint, or other information.

While the example of FIG. 1 was discussed in connection with the joint analysis facility operating on the device(s) 110 and there being a separate client interface on the device 108, it should be appreciated that embodiments are not so limited. In some embodiments, some or all of the functionality discussed above in connection with the device(s) 110 may be implemented on the medical imaging equipment 106 and/or on the client computing device 108. In some embodiments, functionality of the joint analysis facility may be split between medical imaging equipment 106 and client computing device 108, with no joint analysis facility functionality implemented on device(s) 110. In still other embodiments, all functionality of the joint analysis facility and the client interface may be implemented in (e.g., executed on) one or more programs executing on the client computing device 108. Embodiments are not limited to implementing the joint analysis facility in any particular manner.

Examples of ways in which the joint analysis facility may operate are described below in connection with FIG. 2. It should be appreciated that the process 200 is merely illustrative and that other processes are possible.

Prior to the start of the process 200 of FIG. 2, a clinician may identify a patient and a joint to be analyzed. Identifying the patient may include identifying demographic information for the patient, such as age, sex, gender, race, ethnicity, exercise level, or other information. In addition, a clinical exam may be performed on the patient, which may include medical imaging of the joint and/or a physical exam of the joint. The clinical exam may reveal structural and/or functional information regarding the joint, such as structure of the joint and tissues within the joint. The information regarding the joint that is obtained from the clinical exam may include information on location and/or dimensions of anatomical features of the joint, such as anatomical features of bones of the joint. The information regarding the joint that is obtained may also include functional information, such as range of motion information. This may include information on an attainable range of motion for the joint of the patient, such as an amount by which the patient is able to rotate the joint, or an amount by which the patient is able to rotate the joint without pain, or an amount by which the patient is able to rotate the joint with pain the patient considers manageable or below the patient's pain threshold. The functional information regarding the joint may in some cases also include information on translation of bones within the joint, such as an amount by which a bone or an anatomical feature of a bone translates during operation of the joint. Such translation information may be revealed through imaging during operation of the joint by the patient, such as by monitoring movement of bone during operation of the joint while medical imaging is conducted. It should be appreciated that embodiments are not limited to obtaining structural or functional information regarding a joint in any particular manner. In embodiments in which medical imaging is performed, the medical imaging may be received by the joint analysis facility directly from medical imaging equipment or from a picture archiving and communication system (PACS), as input from a clinician, or in another suitable way.

The process 200 begins in block 202, in which the joint analysis facility generates a computer model of the joint to be analyzed. In some embodiments, the computer model may be a default model of the joint, such as a sole default model or one of a set of default models that are selected based on demographic information for the patient (e.g., age, sex, gender, race, ethnicity, exercise level, or other factors). In such a case that a default model is selected, the default model may be customized based on received information regarding the joint of the patient, such as structural information derived from a clinical exam (e.g., physical exam or medical imaging). For example, anatomical features of the joint depicted in the default computer model may be customized based on determined information regarding anatomical features of the patient, such as by customizing the location and dimensions of anatomical features. In other embodiments, rather than operating with default models, received medical imagery for the patient may be used to generate the computer model in block 202. For example, two dimensional (2D) and/or three dimensional (3D) medical imagery of the joint may be processed to generate a 3D computer model of the joint. In some embodiments, techniques for building computer models from existing 2D and/or 3D images may be applied. Such techniques may include performing segmentation on medical imagery to identify different tissues depicted in the medical imagery (e.g., different bones) and create different objects in the computer model for the different tissues. To perform segmentation, object recognition techniques may be applied. Examples of ways in which computer models may be generated are described in further detail below.

In block 204, the joint analysis facility assesses morphology of the joint using the computer model of the joint. In block 204, the facility may conduct a comprehensive assessment of joint morphology, including the tissue structures of the joint. The measured morphological features may be used to assess the risk of injury or to decide on the treatment. The joint analysis facility may conduct comparative analysis between the subject's morphology and matched controls based on factors such as age, sex, gender, race, ethnicity, activity levels, or any other relevant factors. The morphological difference may be used to identify the patterns and locations of the morphological abnormalities or otherwise determine locations and/or dimensions of anatomical features of the joint and determine whether such locations and/or dimensions differ from known locations and/or dimensions of prior patients.

In block 206, the joint analysis facility obtains information on amounts of translation and/or rotation to be simulated with the computer model of the joint that has been generated. The amounts may be expressed in terms of distances to be covered, such as units of distance, or may be degrees of movement such as degrees of rotation. In some embodiments, the amounts may be expressed as ranges of motion.

The information on the amounts may be information that was input by the clinician, provided to the facility, and/or stored. In some cases, the information may be based on a clinical exam of the patient, such as a physical examination and/or medical imagery. In some cases, the amounts of translation and/or rotation may correspond to what a patient is currently able to achieve or currently exhibiting, such as an amount of rotation a patient feels able to perform without unmanageable pain or that a clinician feels from the physical exam is the patient's range of rotation. In other cases, the amounts of translation and/or rotation may correspond to what was identified from medical imagery, such as from detecting amounts of translation of a bone during operation of a joint during medical imaging.

It may be advantageous in some situations for the amounts of translation and/or rotation of block 206 to correspond to the patient's range of motion or operation of the joint. This may help ensure that the simulation that is conducted in block 206 is a simulation of movement by the patient and that the simulation corresponds to the patient's movements. In other situations, though, a clinician may wish to simulate what may occur with more or less translation or rotation than a patient can achieve, and how a patient's joint may operate with ranges of motion other than what a patient is currently able to achieve. Or the amounts may correspond to a previously-identified range of motion of a patient rather than a current range of motion. Embodiments are not limited to operating with specific amounts of translation or rotation, or specific meanings to the amounts.

Examples of amounts of translation and rotation, and ways of determining them, are discussed in more detail below.

In block 208, the joint analysis facility simulates operation of the joint by moving one or more objects of the computer model of the joint. The objects that are moved may include one or more bones of the joint. For example, during simulated operation of a human hip joint, a femur of the hip joint may be moved to simulate walking, running, jumping, or other motions. The operation that is simulated may be set based on the obtained ranges of translation and/or rotation from block 206. For example, during simulated operation, a bone may be rotated an amount that corresponds to the obtained amount of rotation and may be moved laterally an amount that corresponds to the obtained amount of translation. The object(s) of the computer model may be moved to a number of different positions, such as all anatomically possible positions, without user input to select the specific positions that objects of the computer model are moved to over the course of the simulated operation. Instead, the joint analysis facility may be configured with an amount of movement to use for each iteration of multiple iterations over the course of moving along the input amounts of rotation and translation and may move the object(s) of the computer model to a number of different positions automatically based on that configured iterative movement amount. It may be advantageous in embodiments to choose a small amount, to provide granular simulated movement and analyze operation of the joint over the course of the simulation. As should be appreciated from the foregoing, to appropriately simulate operation of a joint, the amount of translation that is obtained in block 206 may be non-zero, such that translation is simulated in block 208, aside from situations in which translation is not to be simulated such as when a patient's bone is improperly less mobile, and no translation is occurring in the patient's joint.

The morphological features measured from block 204 and/or simulation of block 208 may be used by the joint analysis facility to, in block 210, determine information on operation of the joint. The information on operation of the joint may include information on whether the joint is showing normal or pathologic operation and/or normal or pathologic morphology. Pathologic operation may include morphology and/or operation in any manner that shows signs of joint disease or maloperation. This may include size/dimension mismatches, alignment mismatches, impingement of bones during operation, instability of the joint (e.g., under coverage), or other characteristics of joint operation. These assessments may be compared to corresponding parameters measured from demographically matched normal controls to better gauge patient-specific risk and treatment options. To determine the information on operation of the joint, the joint analysis facility may determine whether positions of objects during one or more iterations of the simulating of block 208 indicate that the positions exhibit signs of pathologic operation. As one specific example, if during movement of a bone during the simulation of block 208, the joint analysis facility determines that positions of bones indicate touching or overlap of the bones, this would be a sign of impingement during operation of the joint. As another specific example, during operation of a bone of a ball-and-socket joint the joint analysis facility may determine an amount of the head of the bone (e.g., femoral head) that is covered by the socket of the joint (e.g., the acetabulum) and determine whether the coverage amount indicates instability of the joint that may result from insufficient coverage. Embodiments are not limited to any particular manner of analyzing operation of a joint, as there may be a variety of ways in which a joint can operate improperly and a variety of ways in which operation of a joint may be analyzed.

The information that is determined in block 210 may include, in some embodiments, generating graphical information regarding joint morphology and/or operation of a joint. For example, for an analysis of impingement, the joint analysis facility may generate a graphic showing locations of detected impingement in the computer model. This may be, for example, a heat map showing how much impingement has been detected during the simulating. As another example, with respect to bone coverage, the joint analysis facility can determine regions of the bone that are covered by the socket of the joint during motion and that are not and produce a heat map of coverage. While heat maps have been described, it should be appreciated that other ways of representing joint operation graphically may be used, and that embodiments are not limited to image-based graphics or to graphics at all. As another example, mathematical graphs, tables of numbers, or text reports on joint operation may be generated, examples of which are provided below.

In block 212, the joint analysis facility determines whether the information determined in blocks 204 from morphology assessment and/or 210 from the simulation of block 208 indicates that the joint is improper or operating in an improper manner. Improper operation may include pathologic morphology and/or operation. The facility may make the determination of improper operation in block 212 in a variety of ways, as there are a variety of ways in which pathologic joint morphology and/or operation may be exhibited. In some embodiments, the joint analysis facility may determine whether there has been any morphological abnormalities detected in block 204, or whether there are global or regional difference in joint or tissue morphology compared to a threshold amount and/or compared to matched controls, or other determination. Similarly, the joint analysis facility may determine whether there has been any impingements detected in block 210 through the simulation of block 208, or whether there is more than a threshold amount of joint area that suffers from joint impingement, or other determination. Similarly, for one coverage, the joint analysis facility may determine whether there are any areas of the joint for which low coverage (an indication of potential joint instability, such as dislocation risk) has been detected, or more than a threshold amount of insufficient coverage (e.g., less than a threshold amount of coverage) or more than a threshold amount of positions for which insufficient coverage has been detected.

If no improper operation has been detected, then the process 200 may end. In some embodiments, following process 200, the computer model generated in block 202 and/or the determinations of joint operation may be output by the joint analysis facility. The output may be to storage, such as to an electronic health record of the patient. As another example, the output may be to an interface of the joint analysis facility, such as to an interface operated by the clinician or a colleague of the clinician, or to the patient or another caregiver for the patient. Embodiments are not limited to any particular manner or form of output, in embodiments in which the output is made.

If, however, improper operation is detected in block 212, then in block 214 the joint analysis facility determines whether and/or how to treat the improper operation. In some embodiments, the determination of “whether” to treat may be straightforward, as in the case of improper operation the facility may determine to recommend treatment. In other embodiments, the determination of “whether” to treat may include determining whether one or more criteria are met for treatment, or for particular types of treatment, such as criteria associated with whether treatment may be successful in treating the identified improper operation or whether one treatment may be preferable. In some such embodiments, the determination of how to treat, or what treatment to recommend, may be intertwined with whether to recommend treatment. For example, a determination may be made of whether criteria for one or more treatments are met. As a specific example, the joint analysis facility may determine in block 214 whether to recommend surgery and/or physical therapy for improper joint operation. Each treatment may, in some embodiments, be associated with criteria that indicate whether the treatment is recommended for the improper operation. Physical therapy may be recommended when, for example, simulation of the joint operation indicates that with an increased amount of translation of less than a threshold amount, impingement or coverage issues can be resolved. The threshold amount of translation may be an amount that may be achievable with physical therapy. If such a criterion is met, then physical therapy may be recommended. As another example, if anatomical features of the joint have dimensions that are a cause of impingement or other joint operation difficulties, and by removing and/or reorienting a segment of tissue (e.g., bone) from the joint the joint operation difficulties can be resolved, surgery may be recommended. In some scenarios, in addition to determining whether to recommend surgery, the joint analysis facility may determine a manner in which to conduct the surgery, such as an amount of tissue to remove or adjust to reduce or eliminate impingement or other joint operation difficulties. Examples of ways in which treatment recommendations can be generated, and examples of such treatment recommendations, are provided below.

In block 216, the joint analysis facility outputs the recommendation, such as outputting to an electronic health record, to a user interface, or in other suitable manners. The process 200 then ends. As discussed above, following the process 200, the computer model and/or determined information about the joint may be output following the process 200. In some embodiments in which a recommendation is output, outputting the recommendation may include outputting information regarding morphology and/or operation of the joint, such as a heat map indicating improper operation or other information.

In some embodiments, following the process 200, the joint analysis facility may also store information regarding the computer model of the joint and/or the quantified morphology of the joint and tissues, and/or simulated operation of the joint in a data store of information regarding joints. The information may be stored in connection with information regarding the patient, such as demographic information for the patient, which may have been de-identified or anonymized, or pseudo-anonymized. The information regarding the patient may include demographic information. This data store may include information on joint operation for a number of patients across demographic groups and aid in determining normal and pathologic joint operation across different joints and across different demographic groups, to aid in future determinations of whether joint operation is normal or pathologic. For example, the determination of block 212 may be made in some embodiments in connection with whether joint morphology and/or operation shows a deviation from normal operation for the joint for one or more demographic group(s) of the patient. In some embodiments, the information in the data store may be used to generate models of normal and/or pathologic versions of a joint, such as for the joint and for one or more demographic groups. The model may reveal information on normal and/or pathologic rotation and/or translation of bones of the joint, information on anatomical features of the joint (e.g., normal locations and/or dimensions of anatomical features), or other functional or structural information regarding the joint.

### Example Implementations

Described below are examples of manners in which techniques described herein for analyzing operation of a joint, such as a ball-and-socket joint like a human hip joint, may be implemented. It should be appreciated that embodiments are not limited to operating in accordance with these examples, as other embodiments are possible. Moreover, for ease of description, some techniques are described in connection with different implementations, but those skilled in the art will appreciate that any of the techniques described herein (both below and above) may be implemented together in any suitable combination absent an explicit indication otherwise.

### Examples of Implementation in a “VirtualHip Platform”

VirtualHip is a unique software platform to assist with clinical care of patients with a range of hip disorders, including those leading to limited range of motion (e.g., femoroacetabular impingement (FAI)) and hip instability (e.g., hip dysplasia). Adolescent and young adult patients with FAI or hip instability are at a high risk of hip osteoarthritis (OA) and early need for total joint replacement. Hip preservation surgery is the primary treatment to correct the abnormal morphology to restore normal joint function and minimize the risk of hip OA. Current approaches to guide the hip preservation surgery rely on clinical examinations and 2D assessment of morphological abnormalities. However, these techniques are very surgeon dependent (e.g., better outcomes by more experienced surgeons) and often fail to identify complex morphological abnormalities, thus offer little insight into required treatment. Examples of a comprehensive simulation platform (VirtualHip) are described below to directly and non-invasively study the hip articulation based on 3D imaging (e.g., CT scan) and physical exam data and then to offer guidance on diagnosis and treatment options based on historic data.

FAI syndrome and hip dysplasia are among the most common musculoskeletal conditions and the primary causes of hip pain in the young, active patients. FAI refers to the premature symptomatic contact at the hip joint during motion, which is secondary to a morphologic variation of the proximal femur and/or acetabulum (FIG. 3A). This abnormal contact (impingement) between femur and pelvic leads to reduced range of motion and significant pain as well as labral tear and cartilage degeneration over time. On the other hand, suboptimal acetabulum development (acetabular dysplasia) will result in lack of sufficient femoral head coverage, instability, and ultimately dislocation (FIG. 3B). Both of these conditions are associated with significant short and long-term implications ranging from reduced joint function range of motion to high risk of hip osteoarthritis (OA) and need for total hip replacement earlier in life. It has also been postulated that approximately 50% of all cases of idiopathic hip OA may be linked to an underlying hip joint morphologic variation, such as FAI or dysplasia.

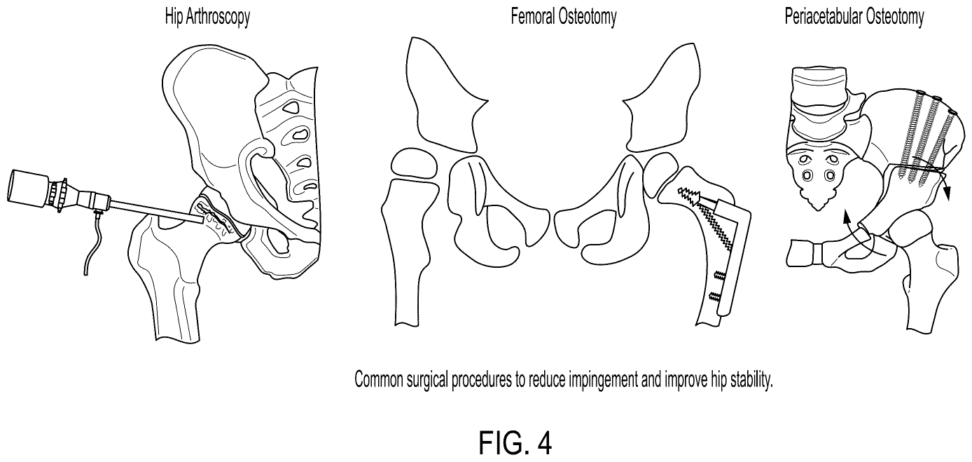

The enormous socioeconomic burden of hip OA highlights the importance of tools for proper diagnosis and treatment of hip injuries leading to lower risk of hip OA later in life. While conservative treatments (e.g., gait modification) can help temporarily relive pain, preservative hip surgery is often required to address underlying abnormal morphology (FIG. 4). Both options conventionally require clear understanding of underlying disease mechanism, impingement pattern, and pattern of acetabular deficiency. Consequently, in accordance with the clinical presentation of the patient, diagnosis of FAI and hip dysplasia are established by assessing these malformations on plain radiographs, computed tomography (CT), or magnetic resonance imaging (MRI). While some morphological abnormalities leading to FAI or hip dysplasia can be detected by looking at the radiographic images and 2D anatomical measurements, assessment of the complex 3D pathomorphology and depiction of the exact impingement or deficiency patterns cannot be accurately achieved with these methods, which can ultimately lead to inferior treatment outcomes. Several groups have shown the variability in successful correction of abnormal morphology in preservative hip surgical techniques (FIG. 4) with higher chance of residual morphological abnormality (e.g., over or under acetabular coverage) in more complex procedures such as periacetabular osteotomy (PAO). Multiple studies have also shown that these residual abnormalities, could lead to inferior outcomes (e.g., joint function), higher prevalence of revision surgery, and elevated risk of hip OA.

Conventionally, discrete 3D modeling and collision detection have been utilized as a means to predict hip impingement and acetabular deficiency, in an effort to minimize the correction errors during preservative hip surgery and to optimize treatment outcomes. Briefly, these techniques involve manually moving the 3D model of the femur (reconstructed from CT or MRI) around a fixed pelvis 3D model (reconstructed from CT or MRI) to identify positions where femur comes in direct contact to pelvis bone or regions of the femoral head not covered by the acetabulum. These simulations can be better than non-computerized analysis techniques in proper characterization and diagnosis of FAI and dysplasia and can help with surgical planning. But despite immense clinical implication, these platforms have not become mainstream, primarily due to limitations in their reliability and effectiveness. These limitations include: A) the non-physiologic assumption of fixed hip center of rotation (allowing for no translation or glide), B) over simplified simulations which do not take into account the soft tissue deformation, C) manual pipeline (i.e., model preparation and analysis) which only allows for assessment of limited hip positions, and D) lack of proper largescale normative and pathological thresholds.

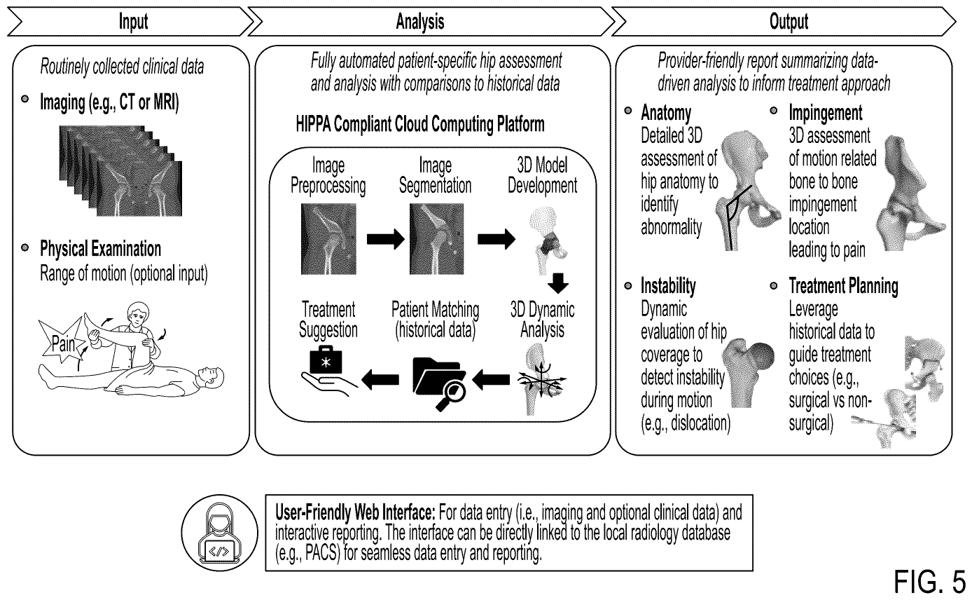

Described herein are examples of the most comprehensive hip assessment platform developed to date, called VirtualHip. This platform can evaluate hip morphology, and impingement and dysplasia (risk and patterns) under all physiologic patient ranges of motion (FIG. 5). Some implementations of the platform may benefit from one or more of the following advantages:

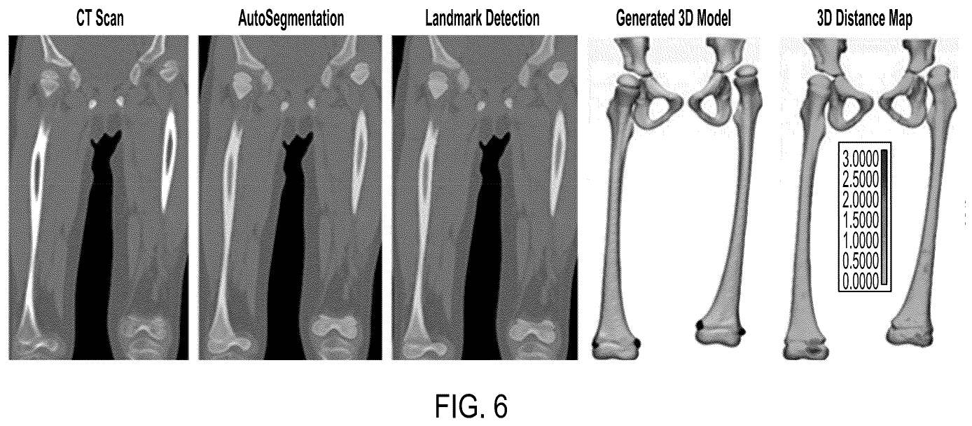

Automatic Workflow: At the current stage, VirtualHip platform includes automatic segmentation of pelvis, femur and sacrum bones followed by automatic model development and landmark detection (FIG. 6). Moreover, VirtualHip is capable automatic anatomy measurements and analysis (i.e., impingement and coverage) under a comprehensive range of rotations and translations.

Comprehensive Assessment: Instead of assessment of hip impingement and coverage under a limited range of motions, VirtualHip will analyze the hip under a wide range of physiological motions (>1 billion combinations of rotations and translations), thus assist with identifying complex impingement and instability patterns which are not detectable using traditional approaches.

Matched Analysis: Owing to its automatic workflow, the VirtualHip has processed thousands of normal and pathological hips, Thus, it can offer matched analysis (e.g., age, sex, race, activity levels, . . . ) based on historic data.

Treatment Planning: VirtualHip can identify the cases which could be treated conservatively (e.g., physical therapy) versus those who require surgical correction. For those deemed to be suitable for conservative treatment, VirtualHip can suggest correction patterns for gait modification and neuromuscular training to minimize impingement. For those needing surgical treatment, VirtualHip can assist with surgical planning by identifying regions which require to be trimmed during osteoplasty/chondroplasty to avoid impingement or bony fragment readjustment patterns (osteotomy) to improve femoral head coverage and/or to minimize impingement.

FIG. 5 describes some functionality of some implementations of VirtualHip. FIG. 5 shows inputs, such as MRI or CT imaging, dynamic ultrasound imaging, and/or physical examination, which can be used to generate an automatic model. FIG. 5 shows an automatic analysis. FIG. 5 shows current outputs of some implementations (i.e., anatomy, impingement zones (density plot), femoral head coverage (orange, the surface of the femoral head covered by acetabulum) and recommended treatment options based on historic data.

### Technical Details of Some VirtualHip Implementations

3D Model Development Module: VirtualHip can utilize an automatic end-to-end pipeline to develop 3D models of the pelvis, femur and sacrum bones from a range of clinical images (e.g., CT or MRI). The model is also capable of automatic identification of key anatomical landmarks (i.e., femoral head, femoral neck, acetabular surface, femoral epicondyles) required to define hip coordinate system, anatomical measurements and dynamic impingement and coverage analysis. The pipeline uses several pre-processing steps (e.g., z-normalization, resampling, . . . ) to prepare the medical images to be segmented by a 3D convolutional neural network (CNN), followed by several post-processing steps (e.g., island identification, over segmentation correction, distance measurement, disconnected component removal, . . . ) to eliminate any potential noise and mis-segmentations. The pipeline is extremely robust capable of analyzing heterogenous clinical images (e.g., different image qualities, acquisition parameters, field of view, . . . ) in an autonomous manner, including direct image query from the clinical database, quality control, 3D segmentation, and landmark detection. We have tested this pipeline on multiple sets of hip images and showed superior performance: mean Dice coefficient of 0.98±0.03 for 3D segmentation and 0.95±0.03 for anatomical landmark detection, including median Hausdorff distance was 0.9±1.7 mm (FIG. 6). The pipeline is developed in Python and will output the 3D models in series of formats including INP and STL among other common formats.

FIG. 6 illustrates output masks and 3D models from the automatic 3D segmentation and landmark detection pipeline. The illustrated 3D distance map indicates the regional differences between automatically developed 3D models vs manually developed ground truth 3D models.

### Morphology Module:

A generated 3D models can be imported to the morphology module, which may be programmed in Matlab, and may be capable of automatic 3D model quality control, model separation (left vs right), coordinate system definition, and measurement of a range of hip morphological features. The module is compatible with Windows, Linux, and Mac operating systems. The input of the module is 3D model of hip joint in any format (e.g., STL and INP formats) generated in the 3D Model Development Module.

### Quality Control and Laterality Labeling

This module can first check the 3D models to identify the laterality (left vs right vs bilateral) and to identify any missing sections (bones or landmarks). The module can also check from any mis-segmentations, in particular extra segmented regions through identifying the number of separate lusters (islands for each part), several volumetric and count-wise removal steps will be taken by the module to remove extra segmentations. The module can log any errors, including missing components. Next, the meta-data from the original imaging data can be used to identify scanner coordinate system. The 3D models can then be aligned to have the body superior-inferior axis in line with the Z-axis (simulating head-to-toe scanning direction). In case of bilateral models, the principal inertia axes of the pelvis model can be calculated, and the second axis can be aligned with inferior-superior (Y) direction. Furthermore, we make sure that the posterior-superior is aligned with Z, and medial-lateral is aligned with X axes. The center of the pelvis model is considered as the local origin of the coordinate system, and left/right label are assigned to models with negative/positive average X.

### Coordinate System

To correctly assess the morphological features, the 3D models should be in a coordinate system. Two different coordinate systems are implemented in the module: pelvis-based and femur-based. In both methods, a sphere is fitted to the femoral head. Also, if the epicondyle model exists, a cylinder is fitted to it. For the pelvis-based coordinate system, similar to the left/right labeling step, the models are initially aligned. The anterior superior iliac spine (ASIS) and posterior superior iliac spine (PSIS) are identified as the most anterior and posterior points of the pelvis model above the pelvic acetabulum. The center of ASISs and PSISs are calculated. The models are aligned such that the line connecting ASISs would be the Z axis, and the normal vector of the plane passes through the ASISs and PSIS center would be the Y axis. The third axis (X) would be calculated based on the cross-product of the Y and Z axes. Furthermore, the center of most anterior points of pubic tubercles (APT) is identified. If the anterior pelvic plane coordinate of the pelvis (pAPP) alignment is selected, the models are rotated around the Z axis such that the line connects pubic tubercles, and ASIS centers align with the Y axis

For the femur-based coordinate system, the normal vector of the plane passes through the center of the fitted femoral head sphere (FHS), and two ends of the axis of the fitted epicondyle cylinder (EPC) would be the X axis. The models are rotated along the X axis such that the line connects the center of the fitted cylinder, and the sphere aligns with the Y axis. The third axis (Z) would be calculated based on the cross-product of the X and Y axes.

For the pelvis-based coordinate system, both left and right pelvis models must be available. On the other hand, for the femur-based coordinate system, the femora epicondyle model must be available. For consistency, the center of the fitted sphere to the femoral head would be the origin regardless of the coordinate system. Furthermore, if the alignment based on both coordinate systems is doable (which depends on the existence of models), the Euler angles between the two coordinate systems are calculated.

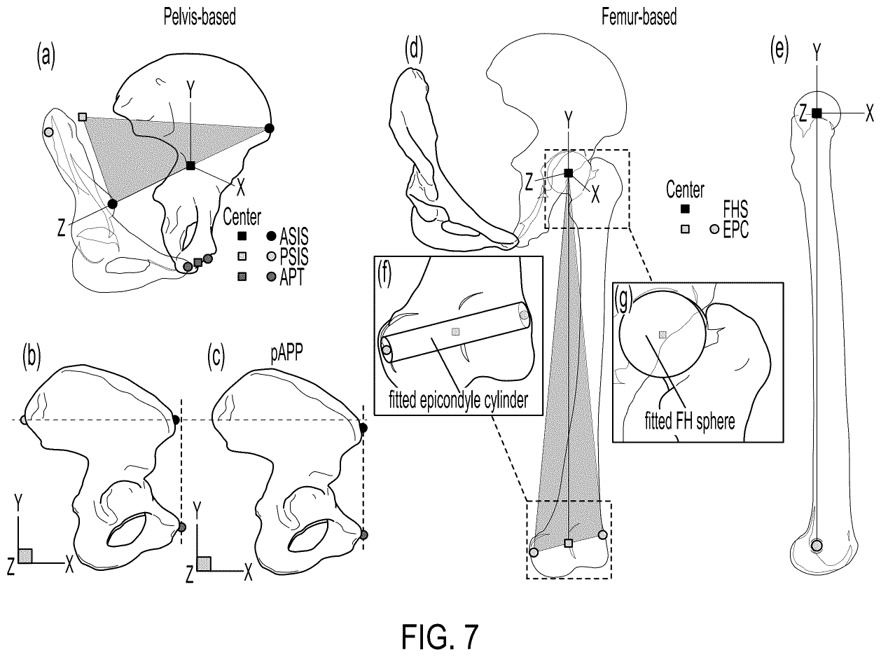

FIG. 7 illustrates pelvis- and femur-based coordinate systems. FIG. 7(a) illustrates, for pelvis-based coordinate system, pelvis models of both sides are needed. ASIS, PSIS, APT, and their centers are specified. Z axis is defined by the line connects ASIS center to right ASIS. The normal vector of the plane passes through ASISs and PSIS center (shown in gray triangle) is Y axis. FIG. 7(b) illustrates the right pelvis is shown in XY plane (sagittal view). The green and blue dashed lines are horizontal and vertical lines, respectively. The horizontal line passes through PSIS and ASIS points shows that they locate on XZ plane. FIG. 7(c) illustrates, if pAPP alignment is selected, the models are rotated around the Z axis such that the APT and ASIS centers would be the Y axis, therefore, the vertical line passes through these points. FIG. 7(d) illustrates, for femur-based coordinate systems, one femur and femoral epicondyle models are needed. The normal vector of the plane passes through EPCs and FHS center (shown in gray triangle) is X axis. FIG. 7(e) illustrates the line connecting EPC and FHS centers is Y axis. FIG. 7(f) illustrates a magnified view of the epicondyle, the fitted cylinder, and its control points. FIG. 7(g) illustrates a magnified view of the femoral head, the fitted sphere, and center of the fitted sphere.

### Femoral Head and Neck, and Pelvic Acetabulum Zones

For specifying femoral head and neck zones on the femur model, the femoral head and neck models are up-scaled based on the vertices' normal vectors. The voxelized models then are generated based on the up-scaled models. The center of femur surface elements which locates within the each voxelized model are identified to define each femoral head and neck zones on the femur 3D model.

For specifying pelvic acetabulum, the distance between vertices of all pelvis and pelvic acetabulum models are calculated. For pelvis vertices, if there is any corresponding vertex on the acetabulum model within a distance smaller than a certain threshold, those vertices of the pelvis belong to the acetabulum zone. The threshold is set based on the average of elements edge length of the pelvis and pelvic acetabulum models.

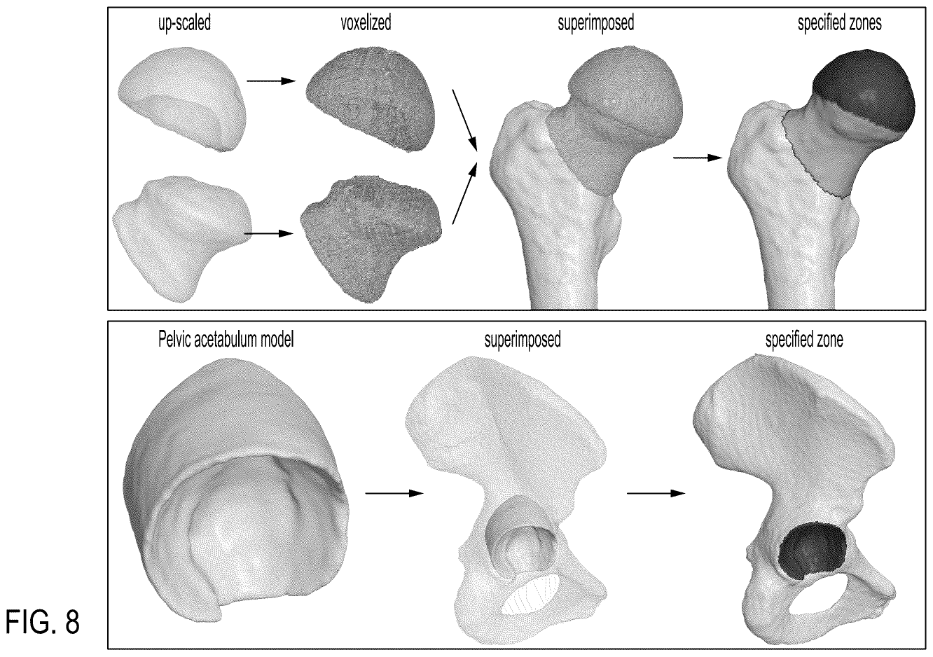

FIG. 8 specifies femoral head and neck (top row) and pelvic acetabulum on the main femur and pelvis models. For femur zones, up-scaled voxelized models of femoral head and neck are superimposed on the femur model, and nodes within the each voxelized mesh are selected. For pelvis zone, pelvic acetabulum model is superimposed on the pelvis model, and the pelvis model vertices which have a distance with pelvic acetabulum model vertices smaller than a certain threshold are selected.

### Femoral Head Axis

Since the femoral head segmentation is available, the femoral head axis is measured based on the principal inertia axes. Furthermore, a plane that separates the femoral head and neck is defined based on the axis and point on the edge of the femoral head. This plane would be physis plane.

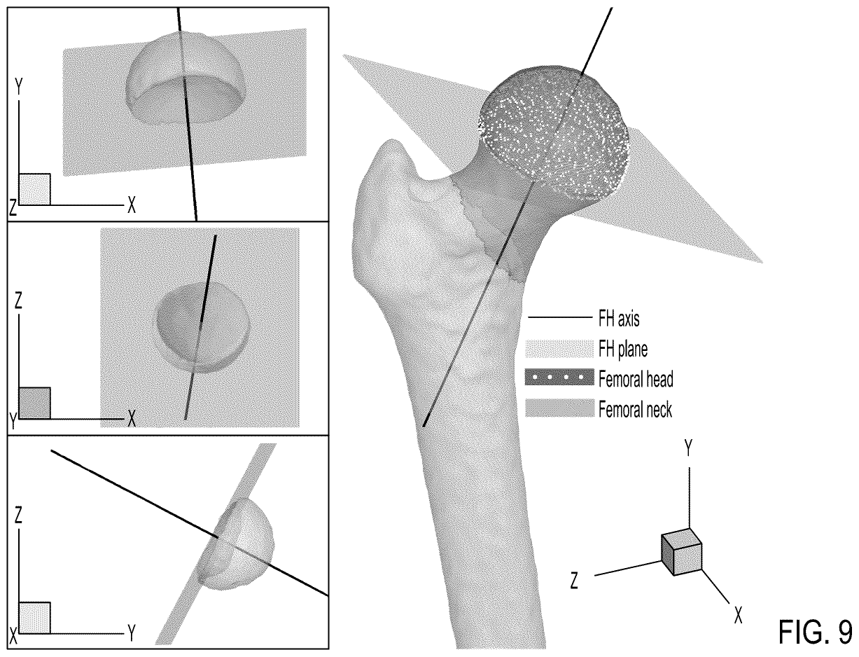

FIG. 9 illustrates determining the femoral head (FH) axis based on the inertia axes of femoral head model. The femoral head model, the femoral head axis, and the plane that separates femoral head and neck (colored in gray) are plotted in three views. This plane would be physis plane. Both femoral head and neck zones are specified on the femur model.

### Femoral Neck Axis

Since the femoral neck segmentation is available, the femoral neck axis is measured based on the top and bottom surfaces of the segmentation (femoral neck model). To find these surfaces, the distance between all vertices of the femur and femoral neck models are calculated. The side surface of the neck model is found based on the measured distances such that for vertices of neck model, if there is any corresponding node on femur within a distance smaller than a certain threshold, those vertices of the neck model belong to the side surface. The top and bottom surfaces are then found by subtracting femoral neck model surface form the side surface. The line that connects the center of bottom to top surfaces forms the neck axis. Moreover, an intersection plane is defined based on femoral neck axis and the point in the middle of two surfaces centers. The points obtained from the intersection of the intersection plane and femoral neck model are used for fitting a circle. The radius of the best fitted circle to the intersection points represents an estimation for the femoral neck radius. Femoral neck length may be defined as the distance between center of top and bottom surfaces. Furthermore, a plane that separates the femoral neck and intertrochanteric area is defined based on the average of bottom surface normal vectors and the central point of the bottom surface.

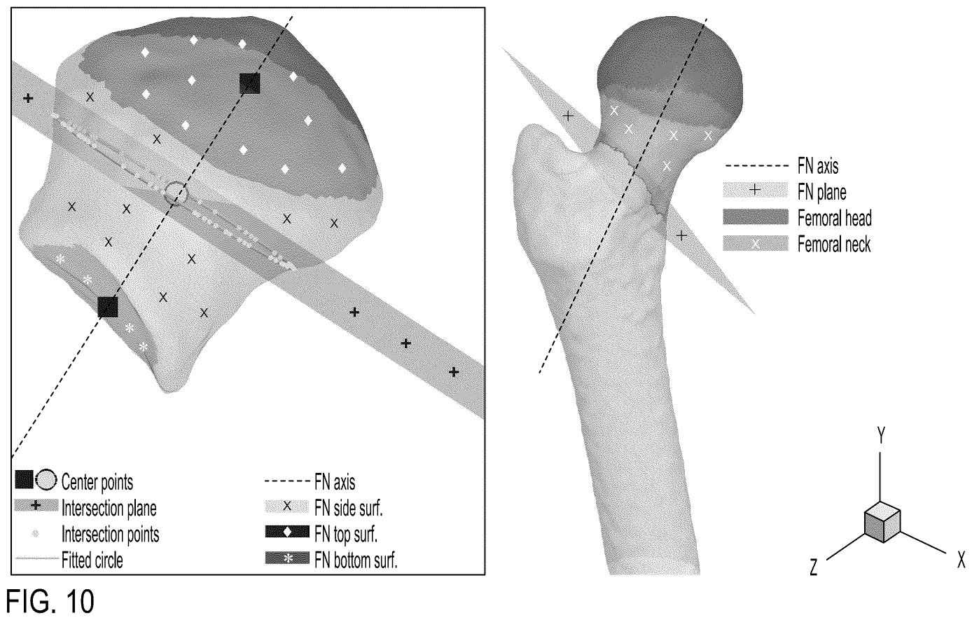

FIG. 10 illustrates determining the femoral neck (FN) axis based on the bottom and top surfaces of the femoral neck model. Since the femur model is hollow (surface model, not volumetric), similar to specifying femoral neck zone on the femur model, the side surface of the femoral neck model is specified. The remaining would be bottom and top surfaces. The femoral neck axis is defined based on the centers of bottom and top surfaces. To estimate the femoral neck diameter, an intersection plane with the normal vector of femoral neck axis is defined in the middle of two center points. The points obtained from the intersection between this plane and the femoral neck model are used to fit a circle. The radius of the best fitted circle is considered as an estimation of the femoral neck radius. The plane that separates the femoral neck and intertrochanteric area is plotted (colored in gray). Both femoral head and neck zones are specified on the femur model.

### Femoral Shaft Axis

The femoral shaft axis is initially defined the principal axis of the region of the femur below the neck and above the condyle region is calculated. Then, planes with normal vector of the principal axis and with 5 mm intervals starting from the bottom of the region are defined along the axis. The intersections of these planes with femur are found which are closed curves, and the center and radius of best fitted circles to each of these curves are obtained. Starting from the bottom, if the change in the radius of the fitted circles exceeds the 20% growth, the corresponding plane would be the plane that separates the femoral shaft and intertrochanteric area. The best linear regression to the fitted circles centers of the shaft region is the final femoral shaft axis.

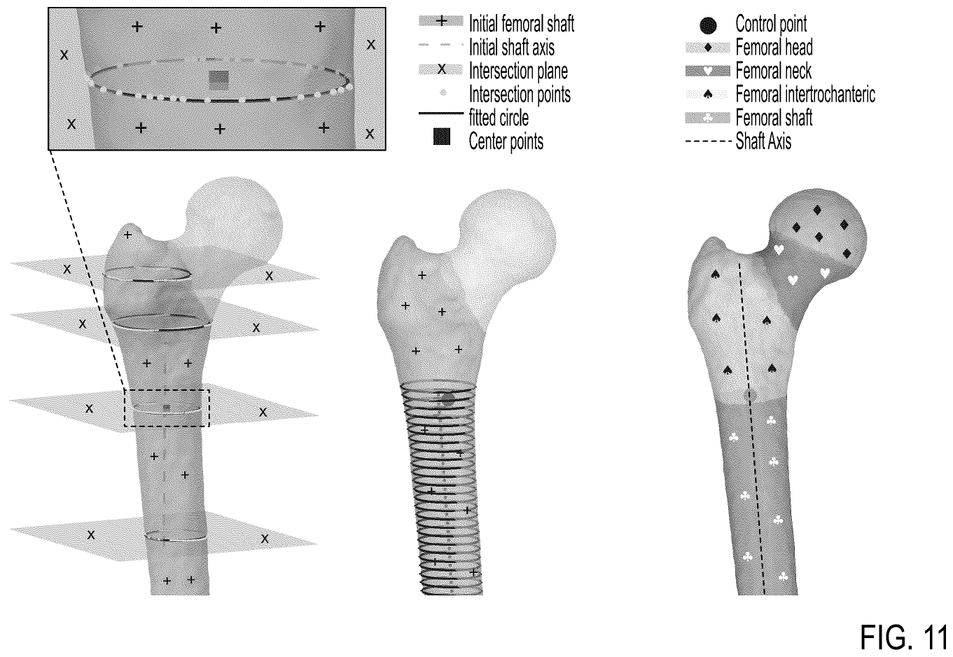

FIG. 11 illustrates determining the shaft axis. The zone on the femur that does not belong femoral head or neck is initially considered as the femoral shaft. Planes (colored in cyan) with normal vector of the initial shaft axis (obtained from principal axes) with 5 mm intervals starting from the bottom of the region are defined along the axis. The intersections of these planes with femur result in intersection point shown by cyan circles. The center and radius of the best fitted circle (shown by solid blue line) to each intersection points group is found. The control point, shown by a red circle, is a section that the radius of the fitted circle is 20% bigger that the bottom circle. This point separate femoral intertrochanteric and femoral shaft zones.

### Posterior Condyle Axis

The posterior condyle axis is defined based on the points on the medial and lateral condyles. To find those points, the condyle region is first defined by cutting the femur model below the shaft region. The condyle region is locally divided into two parts in medial-lateral (Z) direction. The most posterior point of each part is selected, and the line which connects these two points is the posterior condyle axis.

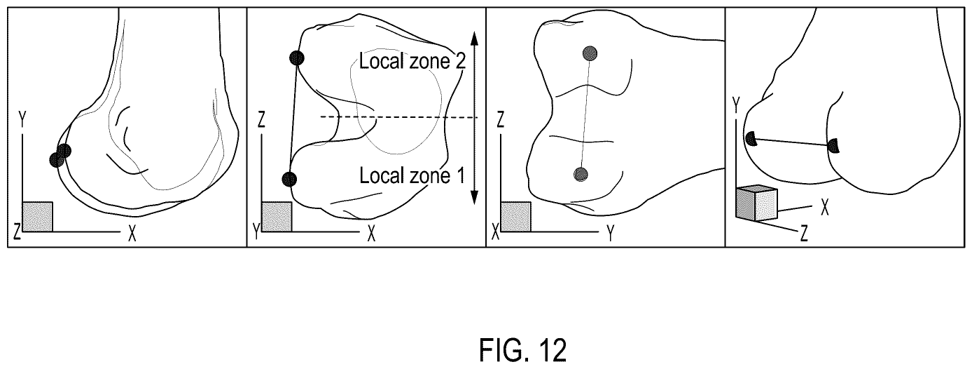

FIG. 12 illustrates determining the posterior condyle axis. The condyle is locally divided into medial and lateral zones. For each zone, the most posterior point (the point with minimum X) is selected. The line connecting these two points is the posterior condyle axis.

### Acetabular Labrum and Clock-Face

To correctly specify acetabular labrum and clock-face, it is necessarily to correctly locate the 12 o'clock. To do that, we first define the acetabulum axis based on the fitted cylinder to the side surface of the pelvic acetabulum model. A plane then is defined using the axis and a point above (on the superior in positive Y direction) of the axis. The intersection of this plane and the pelvic acetabulum model results in a closed curve, the most lateral point on the superior side of the curve is 12 o'clock. The pelvic acetabulum model is locally transferred and rotated such that the axis aligns in Z direction, the center of acetabulum surface locates at (0, 0, 0), and 12 o'clock point locates on X=0 plane with positive Y. The intersection point of the axis and the surface is also found. Other clocks from 1 to 11 o'clock are defined by 30° intervals clockwise rotations starting from 12 o'clock. The acetabular labrum is also specified based on the outer boundary of acetabular surface.

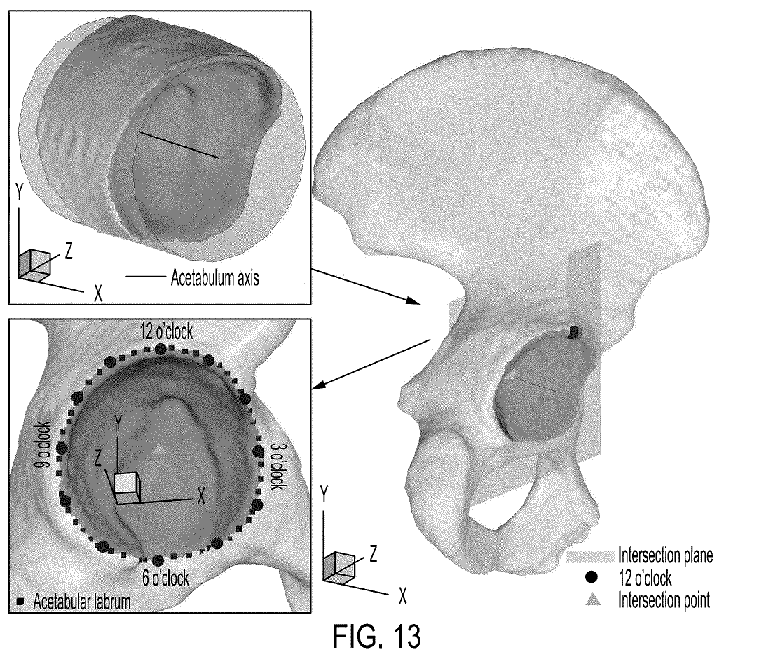

FIG. 13 illustrates specifying acetabular labrum and face-clocks. To find the acetabulum axis, a cylinder is fitted to the side surface of the acetabulum model. The axis of the cylinder would be the axis of the acetabular surface. The acetabulum model and axis are then superimposed on the pelvis model, and the intersection plane is defined based on the axis and a point above (superior) the axis midpoint. The most lateral point on the superior side of the intersection between the intersection plane and pelvic acetabulum model is selected as the 12 o'clock. Other clocks are defined by 30° intervals clockwise rotations starting from 12 o'clock. The outer boundary of the acetabular surface is selected as the acetabular labrum.

### Femoral Head Quadrants and Clock-Face

Similar to acetabular clock-face, it is necessarily to correctly locate the 12 o'clock on femoral head. To do that, we define a plane that passes through the neck axis and a point in the direction of shaft axis vector passes through a point on the neck axis. The intersection of this plane and the femoral head model results in a closed curve. The most lateral point on of the curve is 12 o'clock. To find other clocks, the femoral head model and femoral head surface are locally rotated such that the femoral head axis aligns in-Y direction (i.e., looking to the concave side of the femoral head surface) and 12 o'clock point locates on X=0 plane with positive Z. Femoral head quadrants are also defined by connecting 6 and 12 o'clock, and 3 and 9 o'clock.

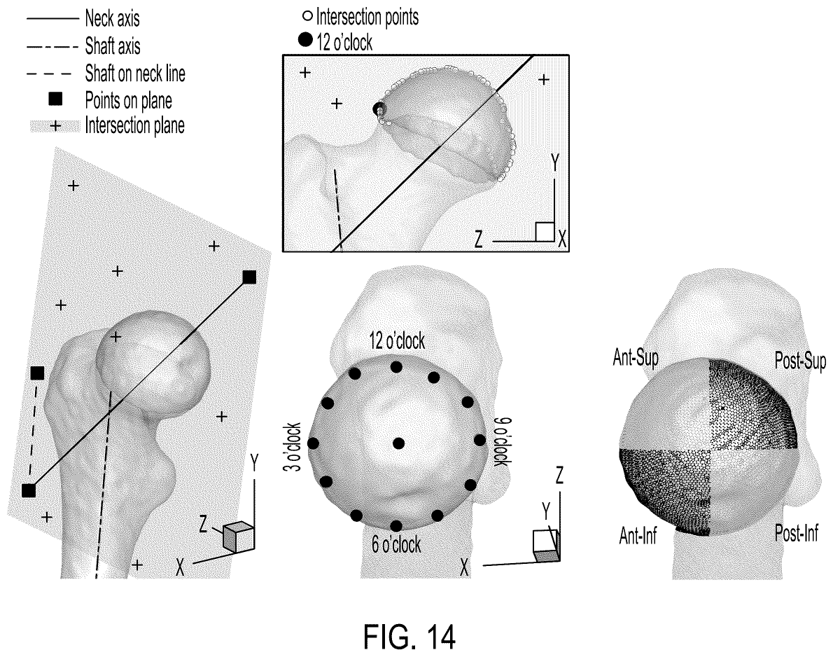

FIG. 14 illustrates determining femoral head quadrants and clock-face. An intersection plane is defined based on two endpoints of the femoral neck axis and a point on the line parallel to femoral shaft axis passing through femoral neck axis. The intersection between the plane and the femoral head is calculated, and the most lateral point is selected as the 12 o'clock. Other clocks and femoral head quadrants are determined accordingly.

### Top Surface of Sacrum

If Sacrum model is available, the proximal S1 end plate will be identified to measure pelvic parameters. To do that, since the surface of the sacrum model might be rough, the model is locally smoothed and aligned with the Y direction using the principal axes. The elements with dominant second component of normal vector are chosen. Among these elements, a central point (the center of chosen elements) on the top part is selected as a reference point. If there is no element above this point, the neighboring elements will be selected and forms a surface, which is the proximal S1 end plate. The average of the normal vectors of the selected surface elements would be the normal vector of the plane tangential to the proximal S1 end plate. If there is any element above the reference point, the search in the closest points to the central point continues until a proper reference point is selected.

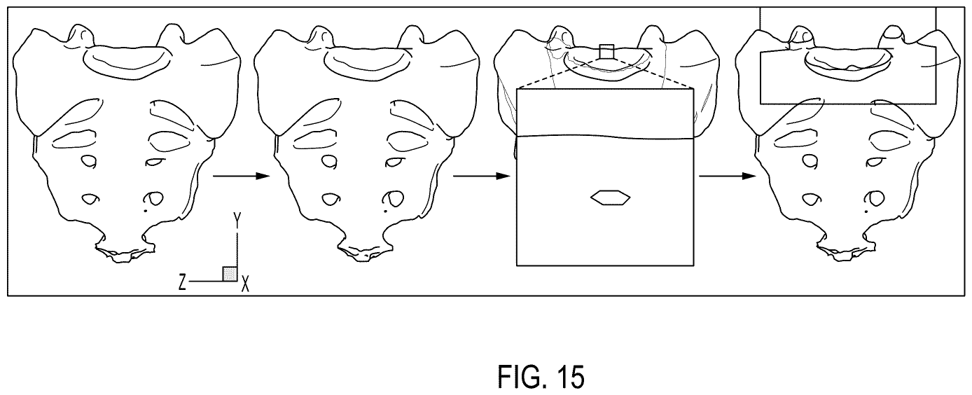

FIG. 15 illustrates finding the sacrum top plane. The sacrum model is smoothed and the top surface is found based on a point on the top surface. The sacral plane then is defined based on the average of the top surface vertices and elements normal vectors.

### Femoral Version

Femoral version will be measured in the following ways:
The angle between the femoral neck axis and posterior condyle axis in XZ plane
The angle between the femoral neck axis and posterior condyle axis in the oblique coronal plane
The angle between the line connecting the center of the femoral shaft (proximal) and center of the femoral head, and posterior condyle axis in the XZ plane
The angle between the line connecting the center of the femoral shaft (proximal) and center of the femoral head, and posterior condyle axis in the oblique coronal plane

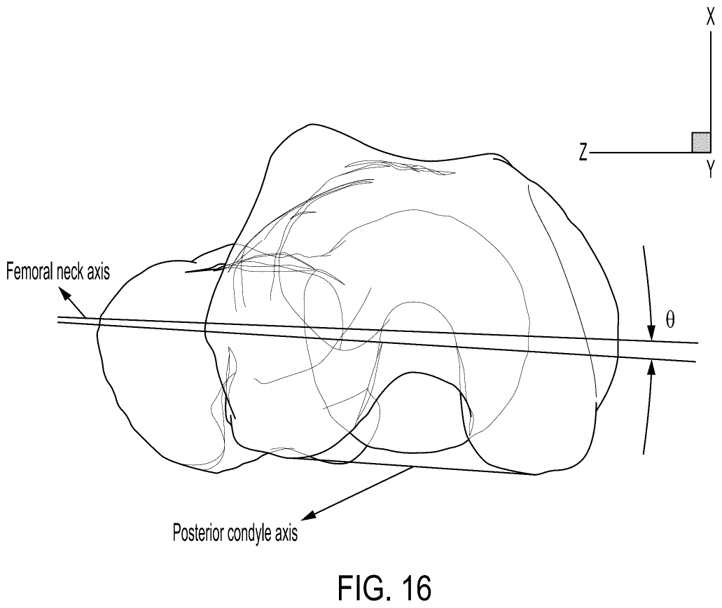

FIG. 16 illustrates femoral version measurement.

### Femoral Offset

Femoral offset is defined as the perpendicular distance from the femoral head center to the femoral shaft axis.

### Femoral Mechanical-Anatomical Angle (Valgus Cut Angle)

The femoral mechanical-anatomical angle (FMA) is defined as the angle between the femoral anatomical axis (femoral shaft axis) and the femoral mechanical axis (a line from the center of condyle to the center of femoral head) in YZ plane.

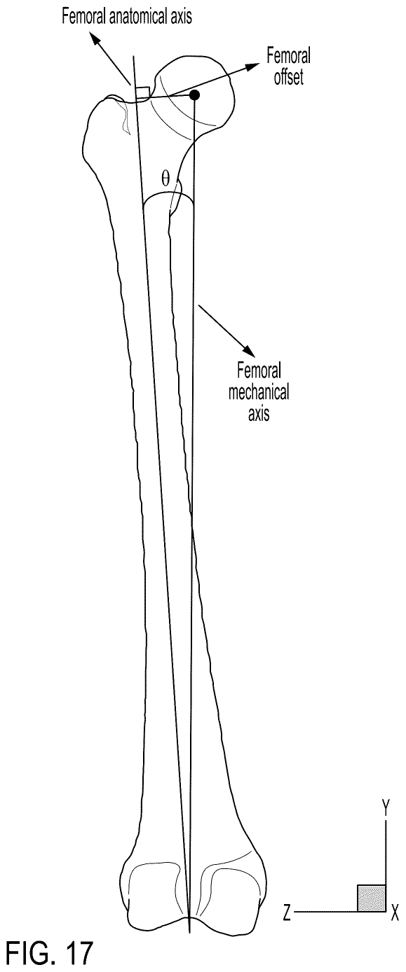

FIG. 17 illustrates femoral mechanical-anatomical angle and femoral offset measurement.

### Femoral Neck-Shaft Angle

Femoral neck-shaft angle (NSA) is defined as the angle between neck axis and shaft axis in YZ plane and in the oblique coronal view.

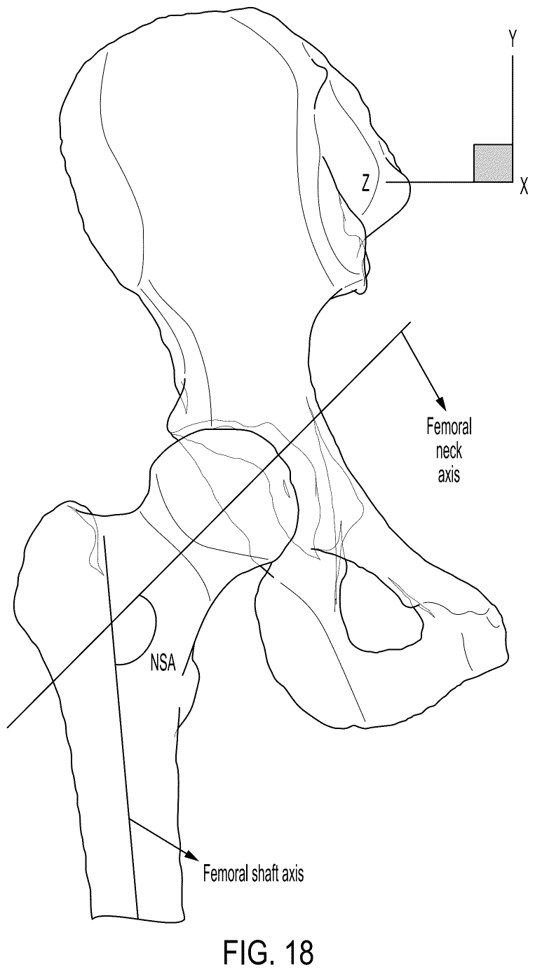

FIG. 18 illustrates femoral neck-shaft angle measurement.

### Femoral Head Neck Tilt Angle

Femoral head-neck tilt angle is defined as the angle between normal vector of the physis plane and the femoral neck axis in coronal, sagittal, and axial views.

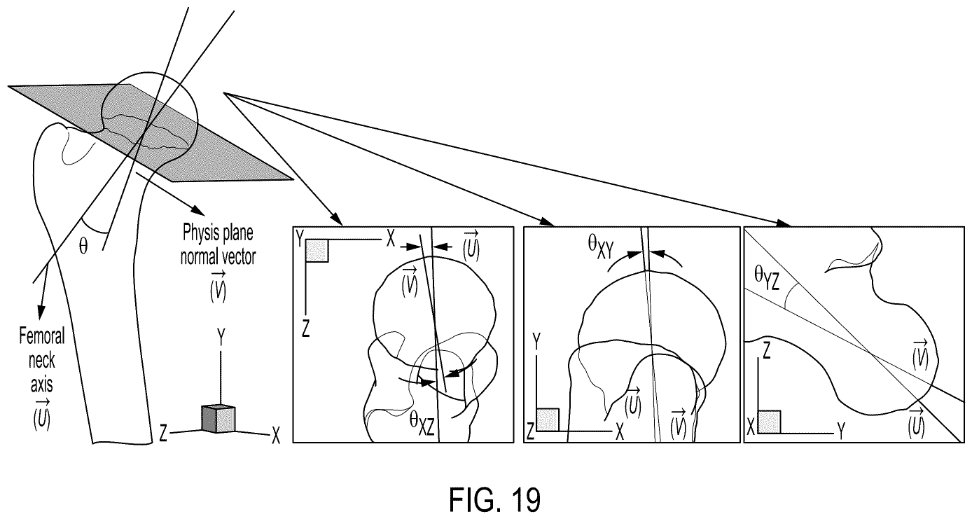

FIG. 19 illustrates femoral head-neck tilt angle measurement in coronal, sagittal, and axial views.

### Femoral Head-Neck Offset and Alpha Angle

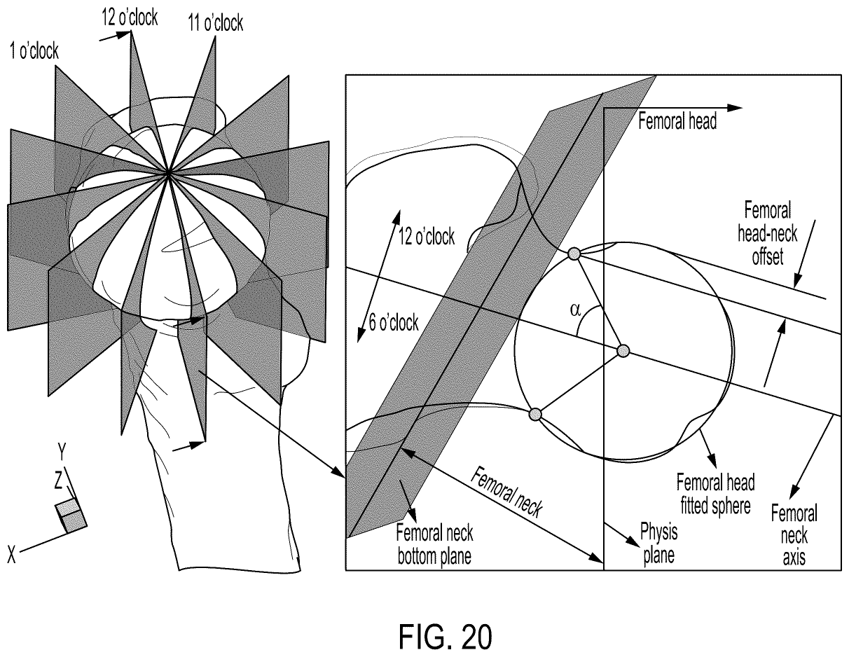

To quantify femoral head-neck offset and Alpha angle, the intersection between the radial cut plane (starting from 12 o'clock and rotating clockwise with 30° intervals around the physis plane axis) and femur is found. The oblique view of 6 and 12 o'clock is shown in FIG. 20. For each clock, the control point is defined as the first radial location on the femur that does not fit in the femoral head fitted sphere, e.g., the intersection between femur and the femoral head fitted sphere. Femoral head-neck offset then is defined as the distance between the lines parallel to the femoral neck axis (1) passing through the control point and (2) tangential to the femoral head fitted sphere. For each clock, Alpha angle is defined as the angle between the femoral neck axis and the line connecting the center of femoral head to the control point.

FIG. 20 illustrates femoral head-neck offset and Alpha angle measurement.

### Center-Edge Angle

At each clock, center-edge angle (CEA) is defined as the angle between the line connecting the center of femoral head to the acetabular clock face and its projection on the XY plane starting from femoral head center. The angle is calculated on the plane of the corresponding clock which contains both lines.

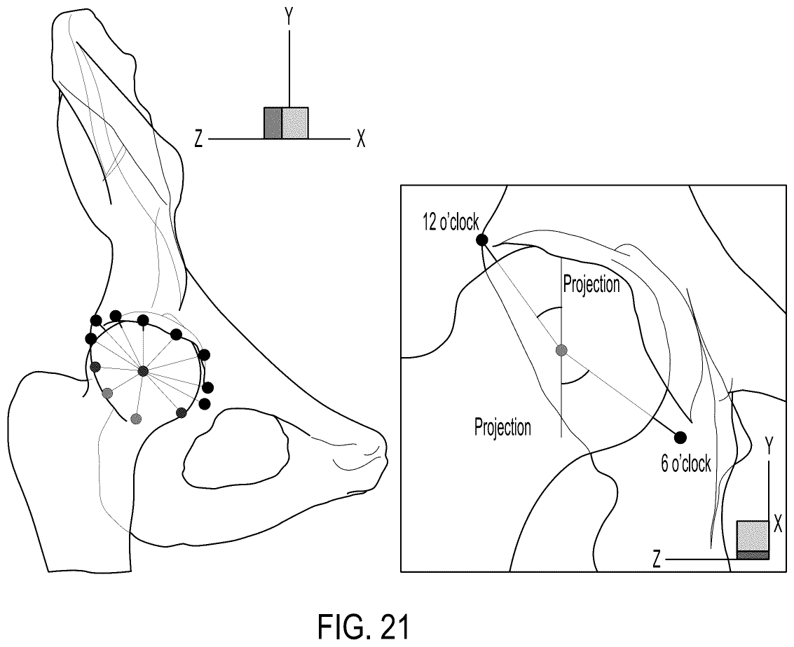

FIG. 21 illustrates CE angle measurement.

### Acetabular Version

Acetabular version is defined as the angle between a line connecting the posterior to anterior acetabular clock face and the line perpendicular to coronal plane passing through the femoral head center in axial view.

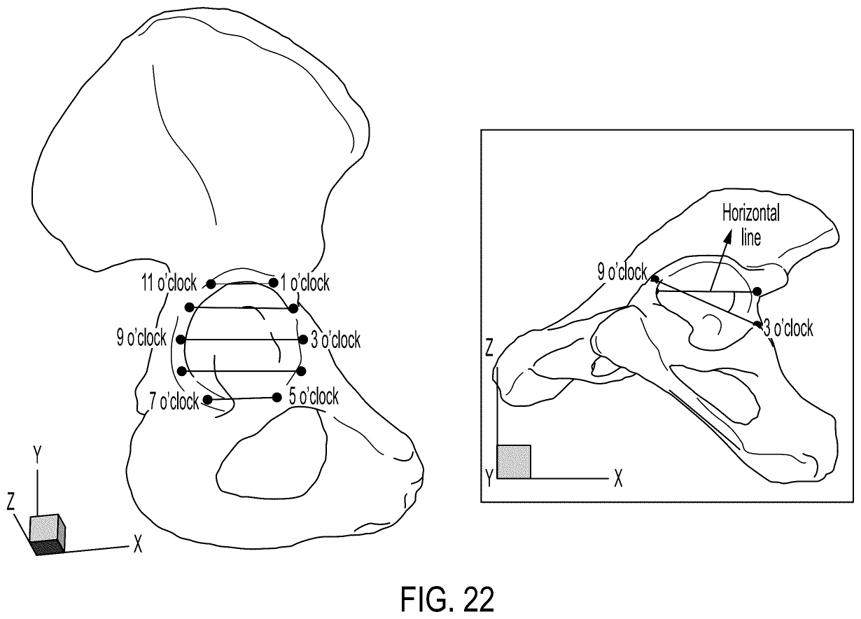

FIG. 22 illustrates acetabular version measurement.

### Acetabular Angle and Acetabular Index

To measure acetabular angle (AA) and acetabular index (AI), reference line is defined by connecting centers of the left and right acetabular surfaces. For each clock, AI then is defined as the angle between the horizontal reference line (Hilgenreiner line as the line connecting the inferior aspect of both triradiate cartilages) and the line connecting acetabular surface center to the clock point in the plane containing both lines. For each pair of clocks (1 and 7 o'clock, 2 and 8 o'clock, and so on), AA is defined as the angle between the horizontal line and the line connecting two clocks in the plane containing both lines.

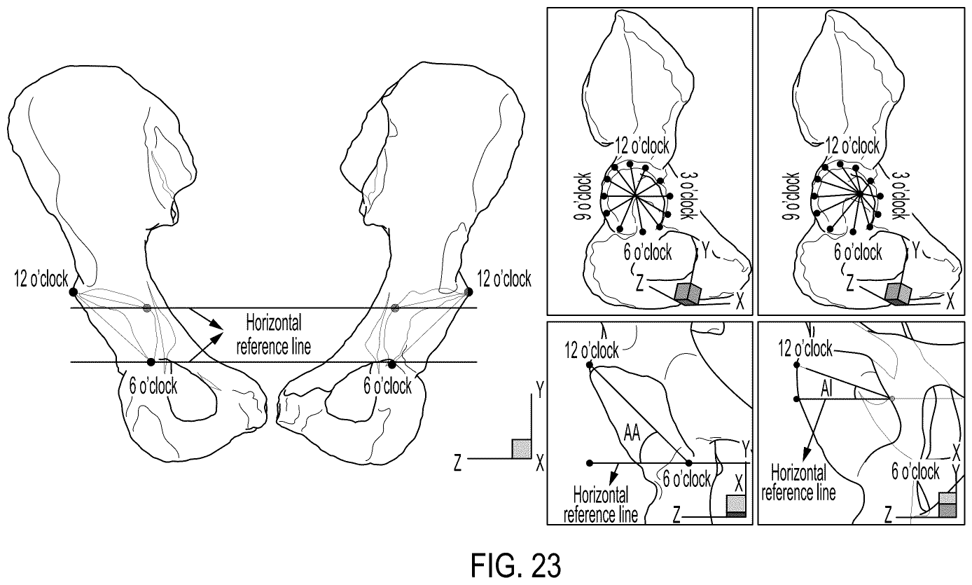

FIG. 23 illustrates acetabular angle (AA) and acetabular index (AI) measurement.

### Migration Percentage

Migration percentage is defined in the coronal view as the lateral amount of the formal head that is not covered by the acetabular surface over the lateral length of the femoral head in percentage.

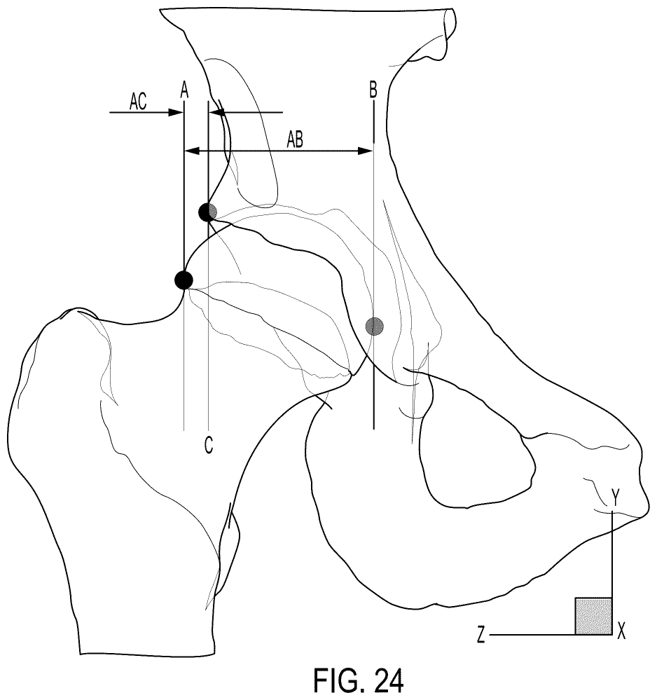

FIG. 24 illustrates migration percentage measurement which is defined as (AC/AB)×100.

### Femoral Epiphyseal Morphology

To find femoral head tubercle location and height (depth) as well as the height of the peripheral cupping, femoral head is locally rotated such that normal vector of femoral head physis plane aligns with the Z axis and 12 o'clock locates at X=0 line with positive Y. The center of the fitted sphere to the femoral head is the origin. (Z component) of inner surface vertices is normalized by the fitted sphere diameter, and the point within the 60% of the diameter with largest depth is defined as the femoral head tubercle. Also, minimum and maximum points of the femoral head in each direction is found, and the depth, height, and width of the femoral head are defined as the distances of the maximum and minimum points respectively in Z, Y, and X directions. In addition to measurements, the module will also generate 2D maps of epiphyseal morphology highlighting the position and size of the epiphyseal tubercle as well as regional size of the peripheral cupping. Such maps are extremely useful to assess the relative sizing of the epiphyseal tubercle and peripheral cupping as primary stabilizer of the head-neck junction.

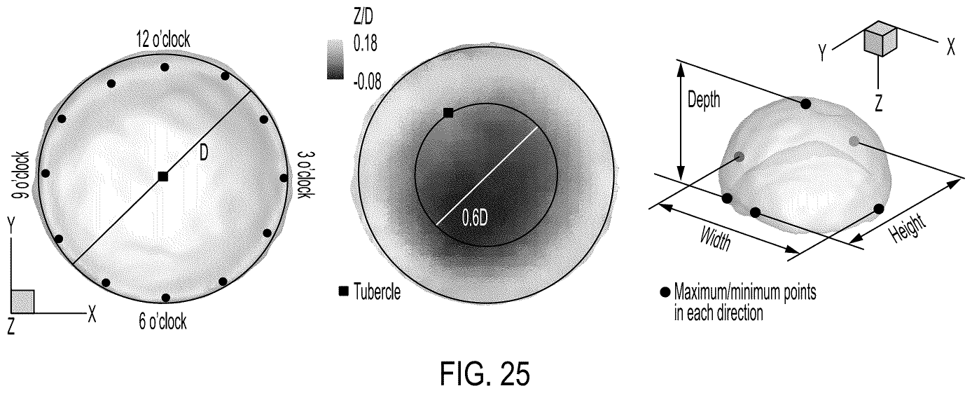

FIG. 25 illustrates femoral epiphyseal morphology and dimensions.

### Acetabular Surface Thickness Map and Acetabulum Dimensions

To find acetabular surface thickness, the acetabulum is locally rotated such that acetabular surface axis aligns with the Z axis and 12 o'clock locates at X-0 line with positive Y. The intersection point of the acetabulum axis and the acetabular surface is also the local origin. Depth (Z component) of acetabular surface vertices is normalized by the fitted sphere diameter. In addition to measurements, the module will also generate 2D maps of acetabular surface highlighting the relative size of acetabular facets (walls). Such maps are extremely useful to assess the relative coverage of the acetabulum across different regions which directly impacts the stability of the hip joint.

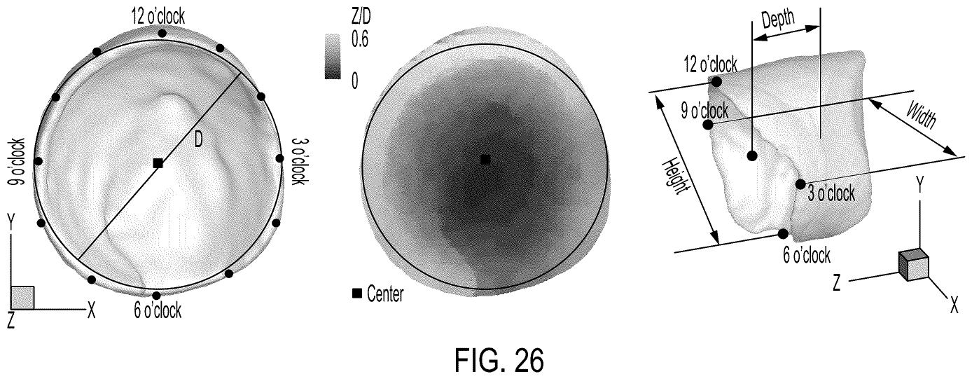

FIG. 26 illustrates acetabular surface thickness map and acetabulum dimensions.

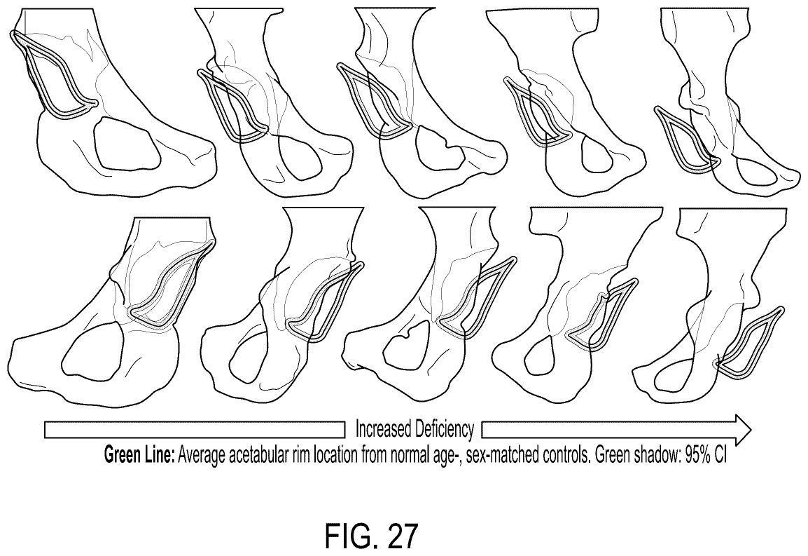

To define normal acetabular rim for each asymptomatic cohort (same age and sex), the acetabulum surfaces were locally transformed in XY plane (the surface axis aligns with positive Z direction) with intersection point locates on the origin, and 12 o'clock locates in positive Y direction. After identifying points on the acetabular rim of each surface, the points are radially classified in 72 groups (5° step). For each group, the points from all surfaces were merged, and outlier points were found and excluded from the point cloud. The mean point as well as 10%, 90% confidence intervals were calculated. A cylinder is then fitted to the point cloud. The mean point and fitted cylinder of each radial section are connected to each other to form a closed curve (mean points) and surface, respectively. In FIG. 27, the green solid line represents the average position of the acetabular rim from a large cohort of normal age- and sex-match controls. The Green shade represents the 90% confidence interval of normal acetabular rim position. In each case, the medial/lateral deviation of the acetabular rim from the normal acetabular rim shows under/over coverage of the femoral head.

FIG. 27 illustrates 3D assessment of acetabular deficiency compared to demographically matched controls

### Sacro-Pelvic Parameters

To evaluate the sacro-pelvic parameters, the proximal S1 end plate (pS1) and the femoral head center are used. The measurements are defined in sagittal view (XY plane). Pelvic incidence (PI), which represents the relative position of the pS1 to the femoral head, is defined as the angle between the line connecting the pS1 and femoral head centers, and the line in direction of pS1 normal vector passing through the pS1 center. Sacral slope (SS) is defined as the angle between the pS1 and anterior-posterior (X) direction. Pelvic tilt (PT) is defined as the angle between the line connecting the femoral head center to pS1 center and the inferior-superior (Y) direction.

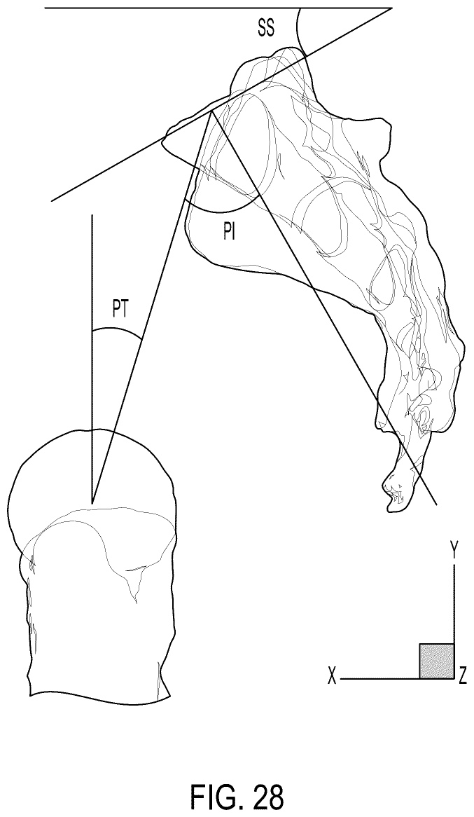

FIG. 28 illustrates sacro-Pelvic parameters measurement.

### Joint Space Width:

The distance between the acetabulum lunate surface and the femoral head surface determines the joint space width. To identify the lunate surface, the elements medial to the intersection point (of the acetabulum axis and surface), as well as elements between 5 and 7 o'clock are removed. Radial cuts are performed and for each cut, the point with the critical point of curve (resulted from the intersection of the radial cut plane and acetabulum) is identified. The average of the critical points, 6 o'clock, and the intersection point, and the axis of acetabulum are used to define a plane. The elements medial to this plane are also removed. For each node of the lunate surface, the minimum distance with the femoral head surface is calculated as the joint space width.

The median values of the joint space width are calculated across the following regions:
Overall: the whole lunate surface
Anterior: the zone between 2 and 5 o'clock
Superior: the zone between 10 and 2 o'clock
Posterior: the zone between 7 and 10 o'clock

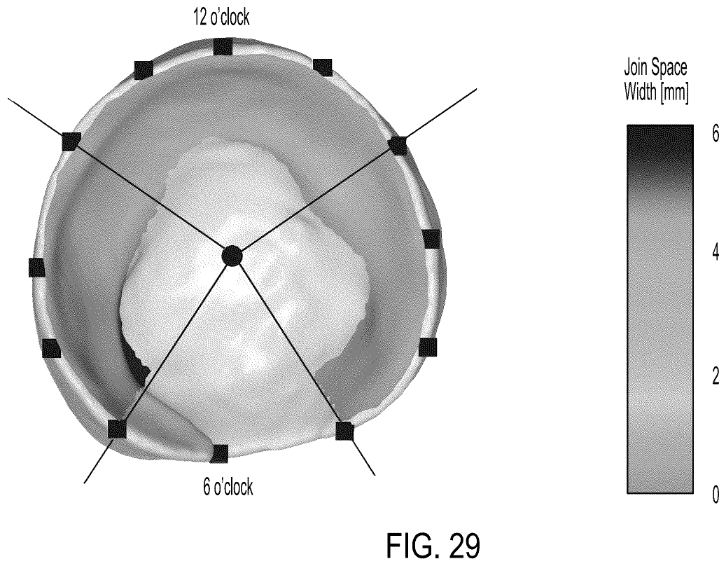

FIG. 29 illustrates joint space width measurement.

We have validated these measurements against manual measurements in 120 normal and pathologic hips and have shown strong agreements between manual measurements and those done by the VirtualHip, with measurement error less than 2 mm and 5 degrees, which are below error ranges reported among human examiners.

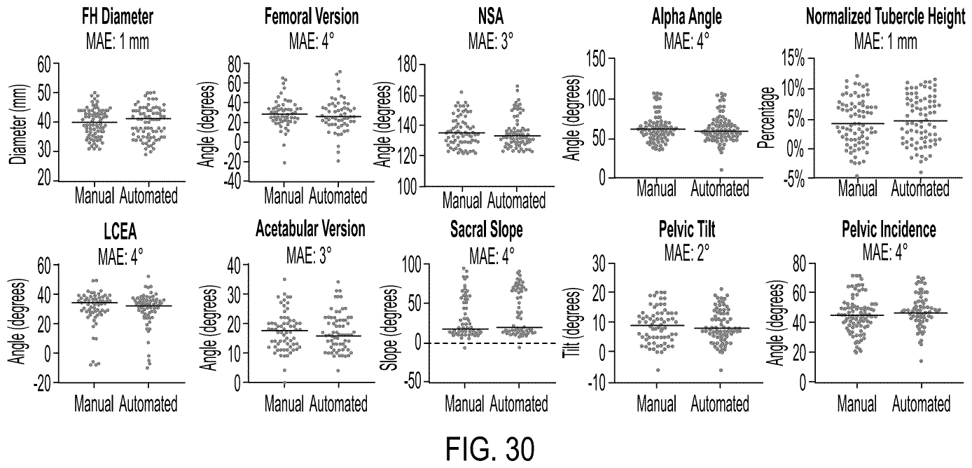

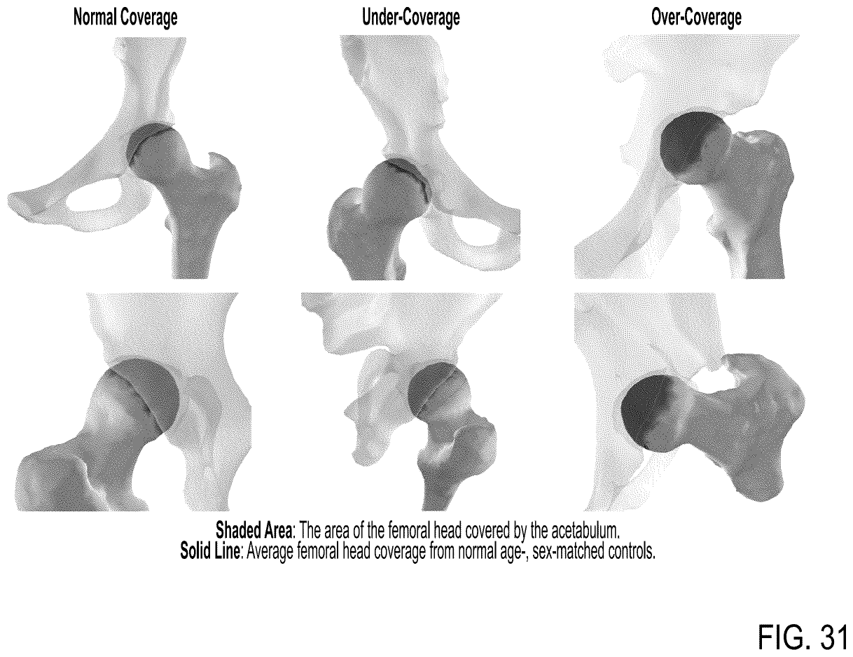

FIG. 30 illustrates the comparisons between experienced human examiner (manual) and VirtualHip (automated) in measuring key morphological features of the hip joint.

Most importantly, the automatic measurements done by VirtualHip were significantly more consistent (test vs retest) vs those done manually by the human examiner. In contrast to the existing normal/pathologic thresholds, which have been mainly obtained from 2D x-ray measurements done limited number of subjects, VirtualHip offers detailed 3D thresholds (adjusted for multiple demographic factors e.g., age, sex, race, . . . ) owing to its autonomous nature to process large amount of historic data. Thus, VirtualHip offers a more accurate insight into hip morphological abnormality which can directly impact the diagnosis and treatment planning.

### Comprehensive Hip Impingement (Collision Detection) Assessment Module

The collision between the femur and pelvis is detected based on triangle-triangle intersection. The femur and pelvis models are formed based on fine triangular surface mesh. The collision simulation is based on 6-DOF (3 rotations and 3 translations). In addition to generic range of motion, the patient specific range of motion can be imported by the user from the clinical examination or dynamic ultrasound. The translations allow to account for changes hip center of rotation due to glide (non-spherical contact) and potential soft-tissue deformation. In the full physiological range of motion (i.e., flexion/extension, abduction/adduction, internal/external rotation, and translation (glide) in medial/lateral, inferior/superior, and anterior/posterior directions), the femur model is transferred to the new position using fine intervals. At each position of femur, if any element of femur model has intersection with any element of pelvis model, those elements of both models are marked as the collision. The module can also classify the collisions into intra-articular and extra-articular depending on the region of collision. The algorithm would also use the distances and collisions to identify contact between the bones to avoid any penetration. The module can report the collision across the whole joint or within each region as automatically defined by the morphology module (e.g., clockface regions). Since both rotation and translation intervals are fine, the possibility of models' penetration without collision does not. Considering physiological range of motions and fine intervals, more than 1 billion of possible positions are tested for collision. We have shown that assumption of fixed hip center of rotation and lack of accounting for hip translation as done in the existing simulation packages results in significant errors in collision detection.

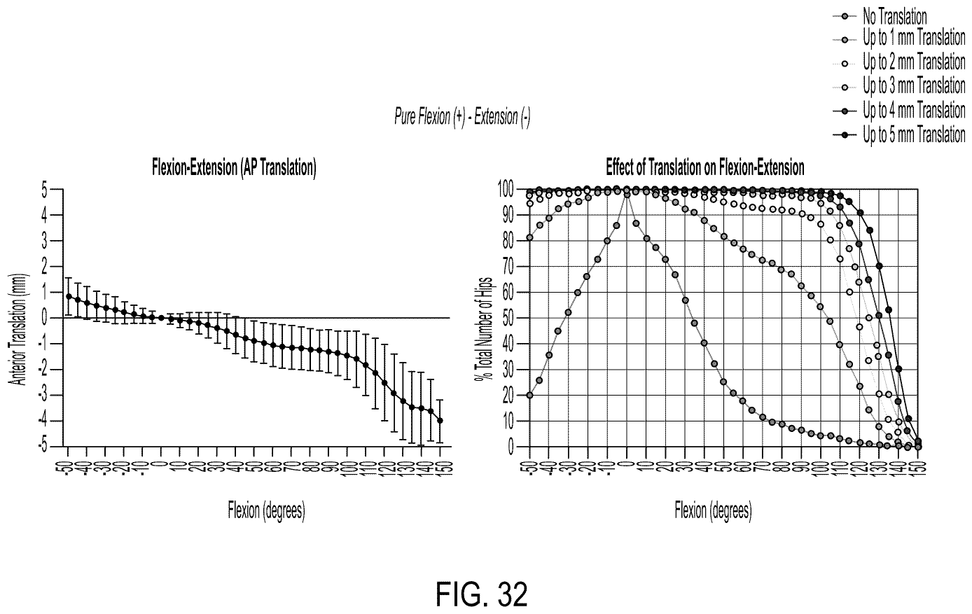

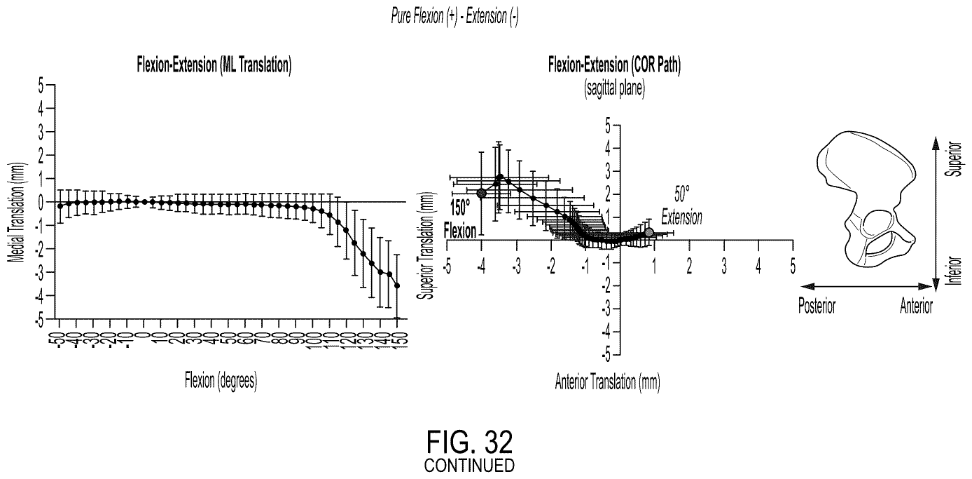

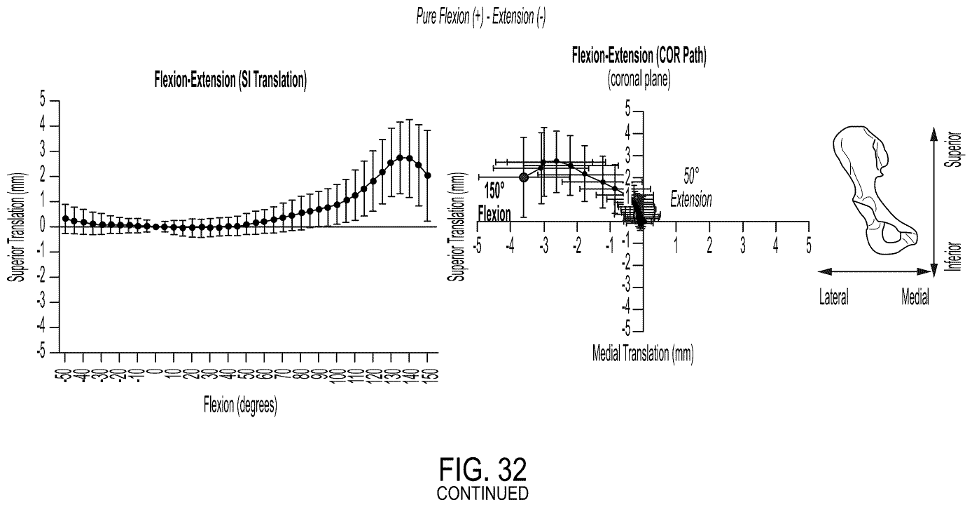

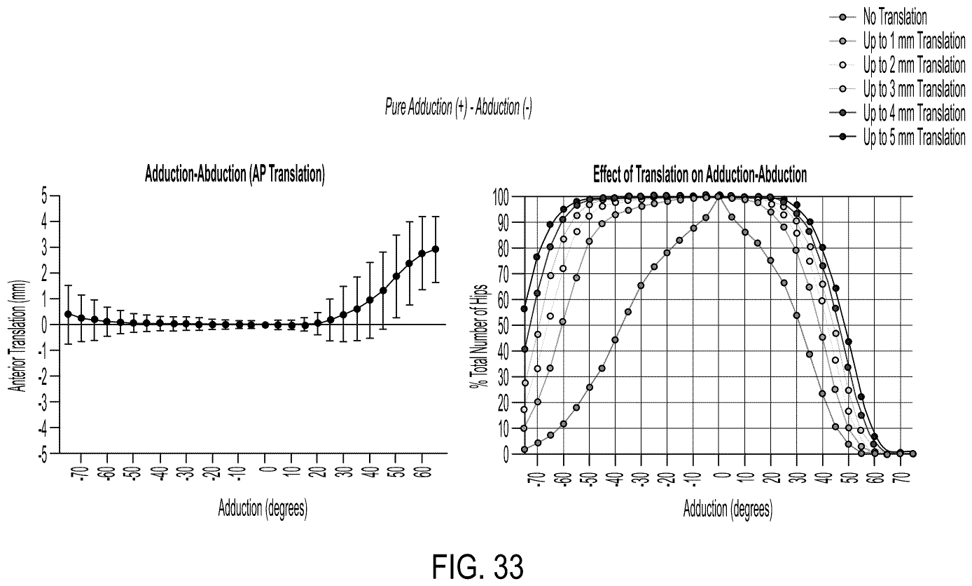

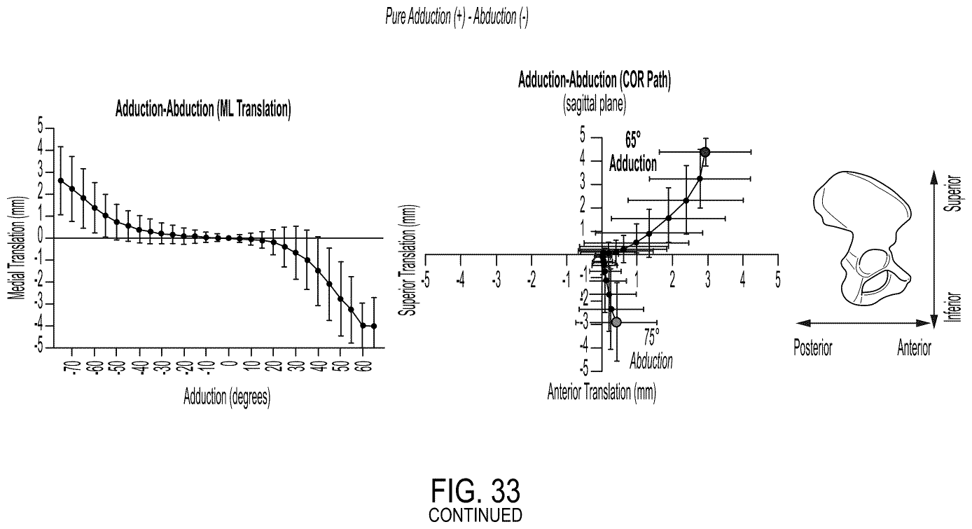

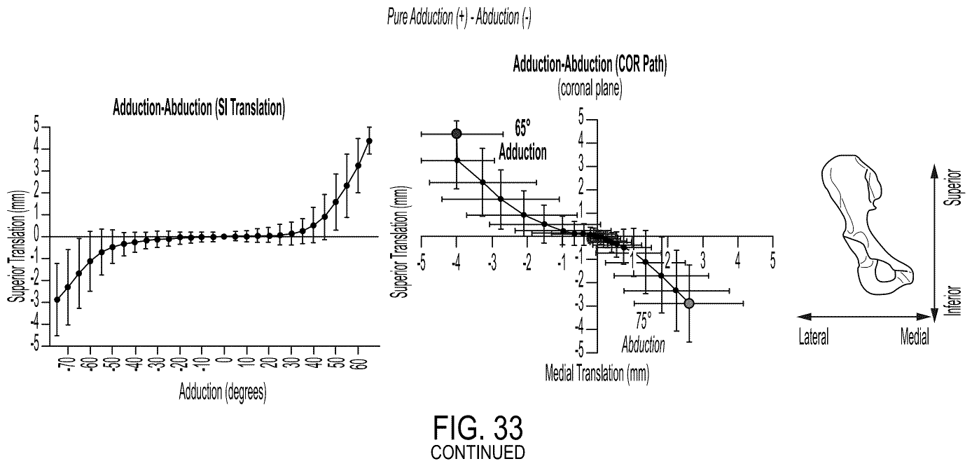

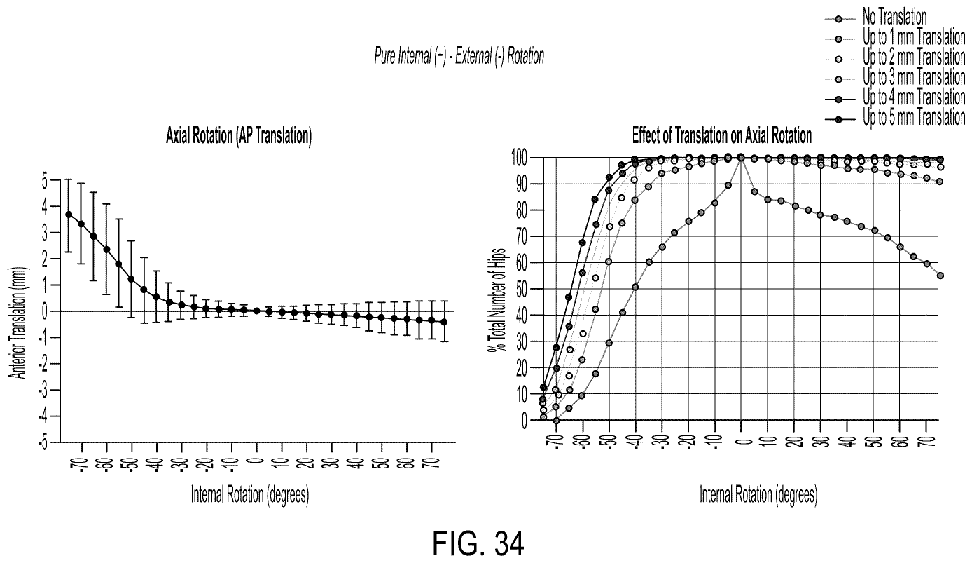

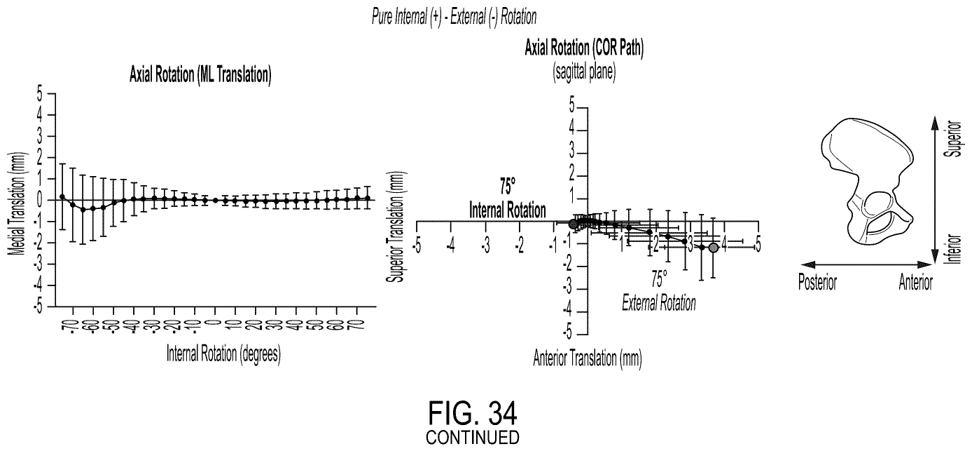

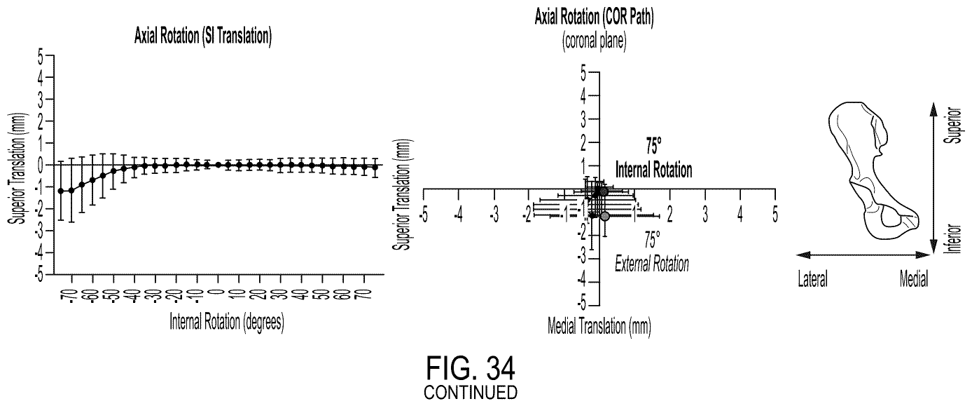

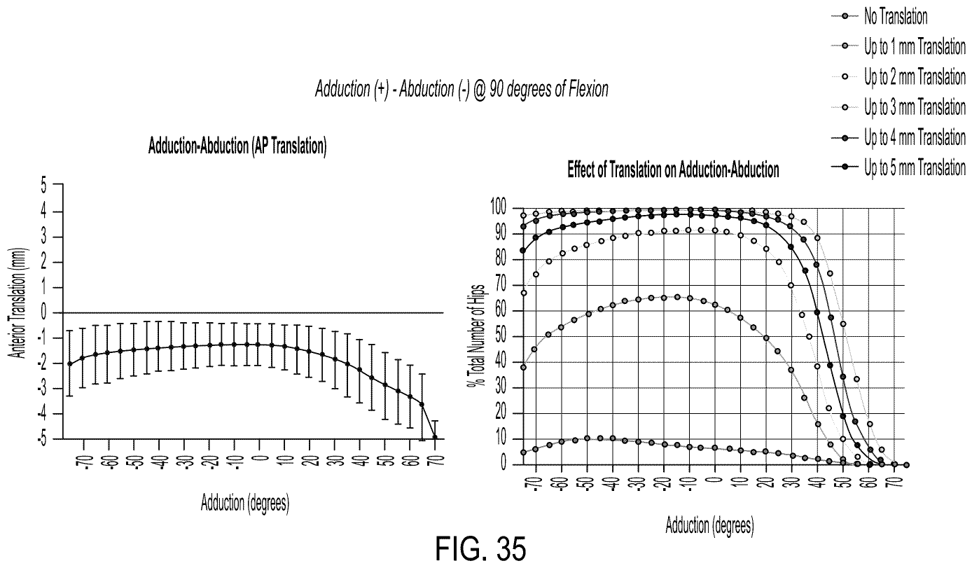

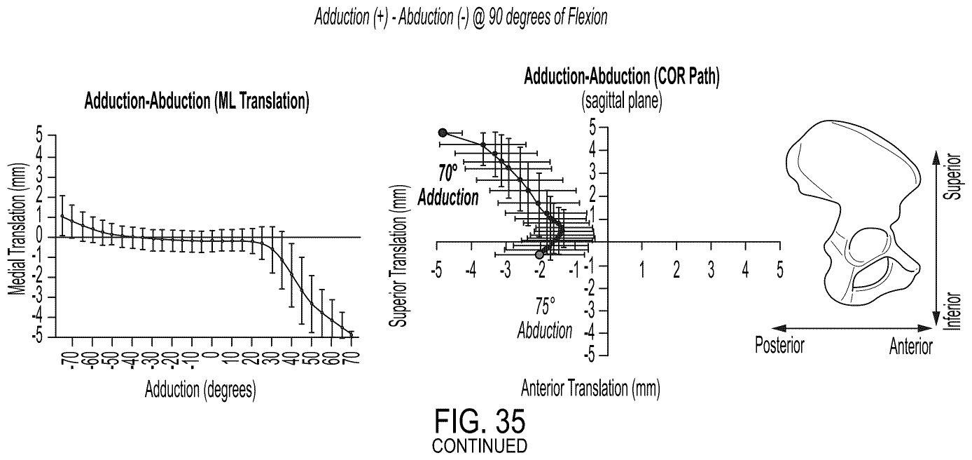

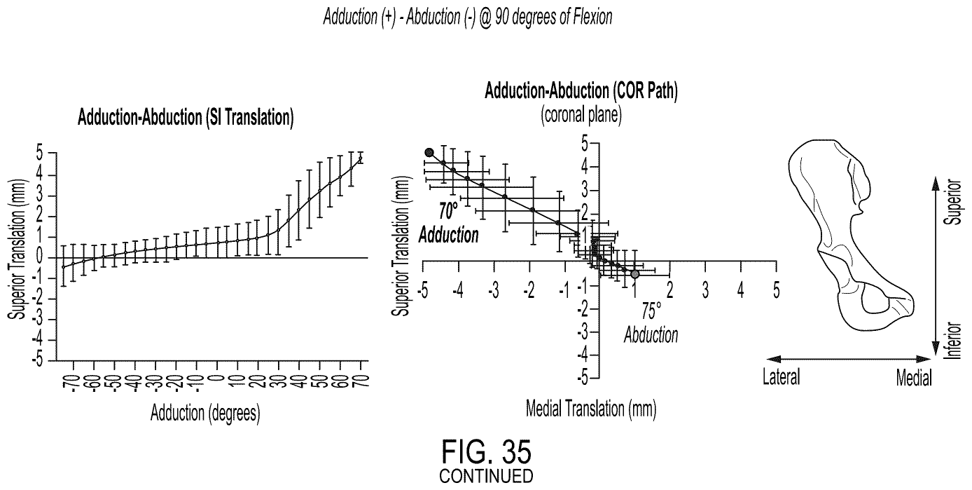

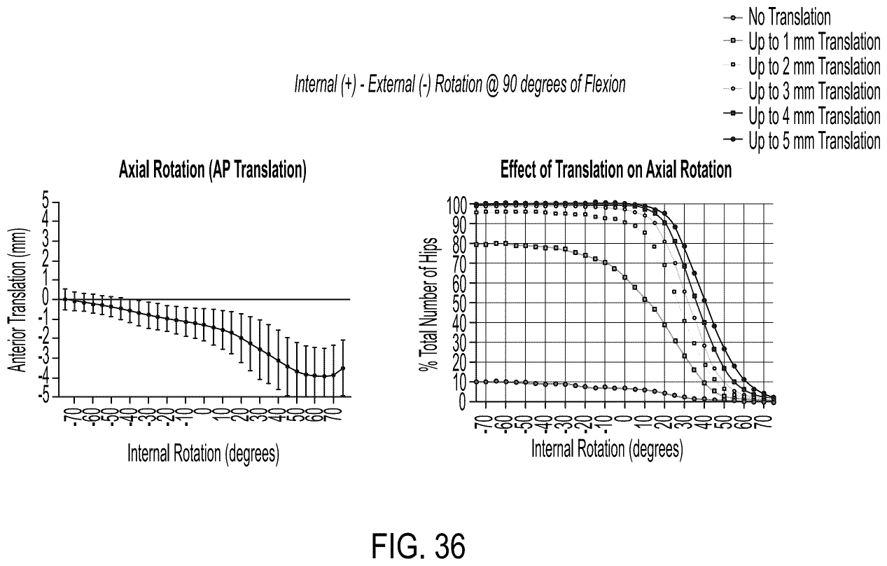

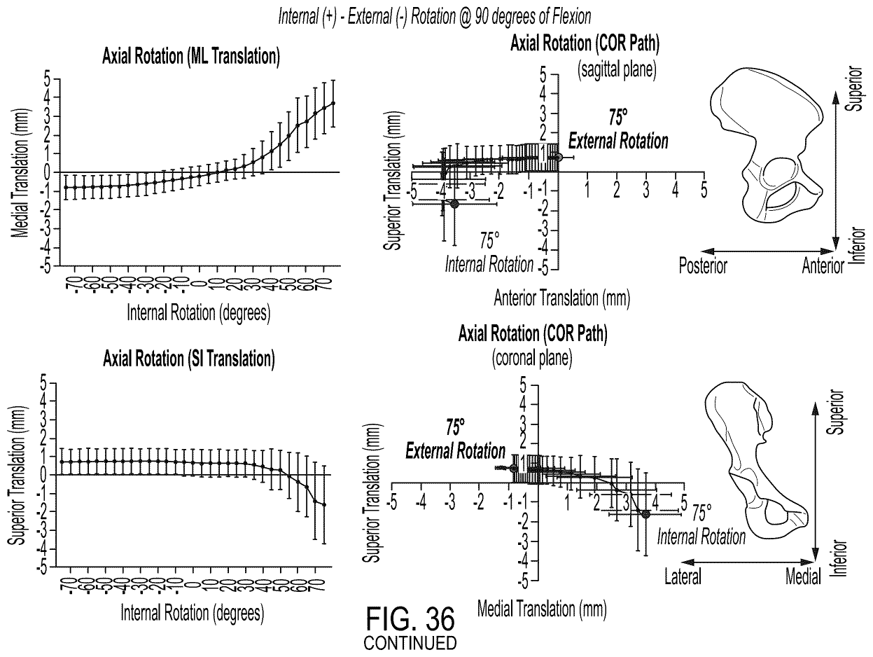

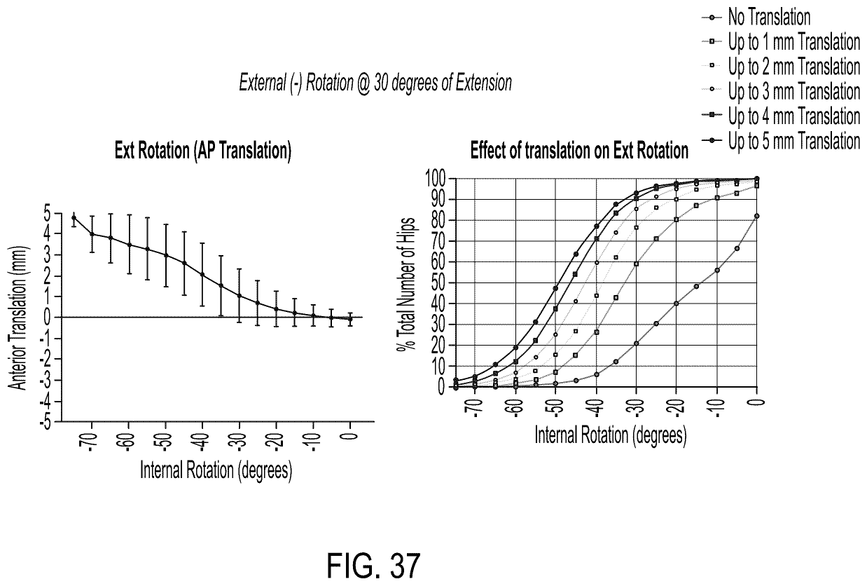

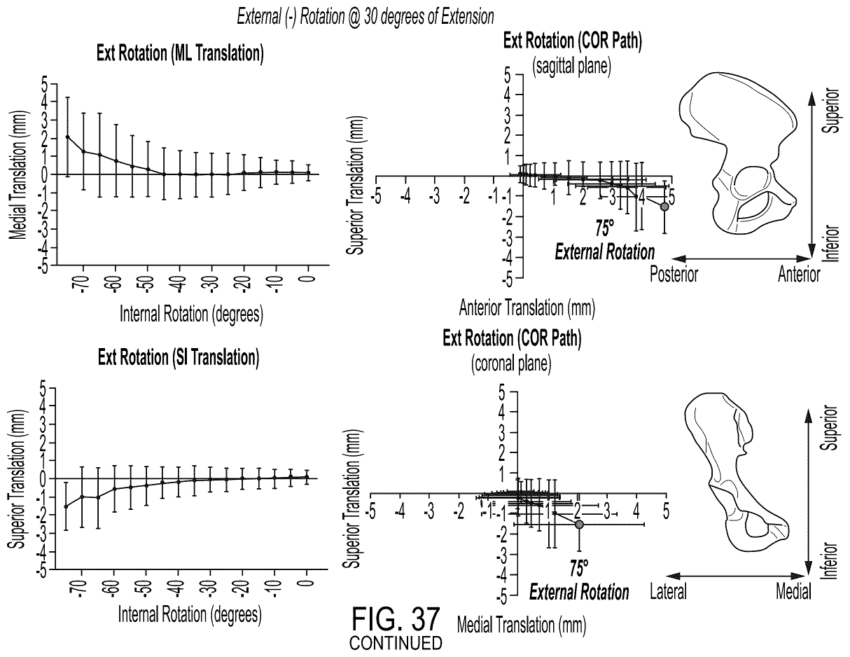

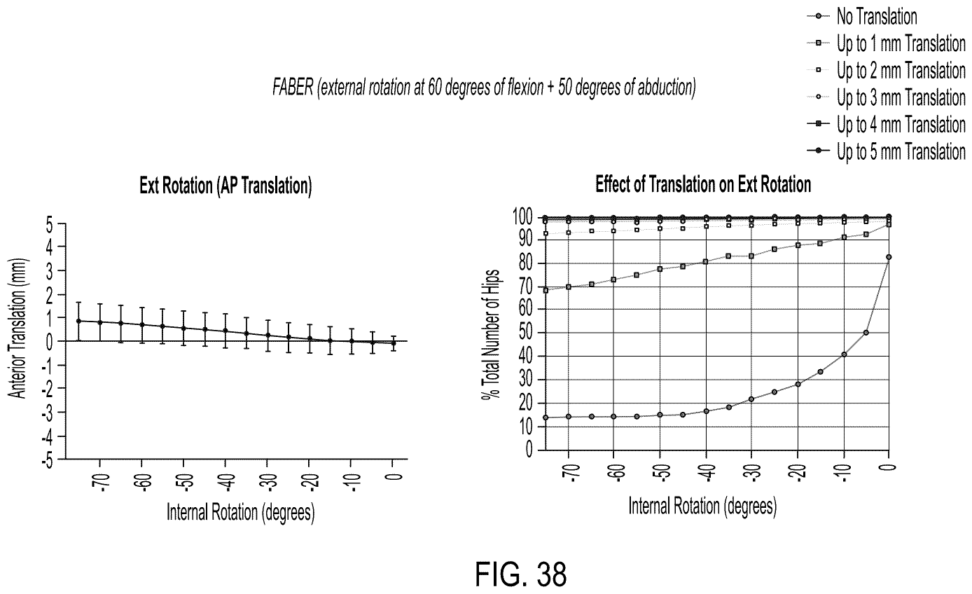

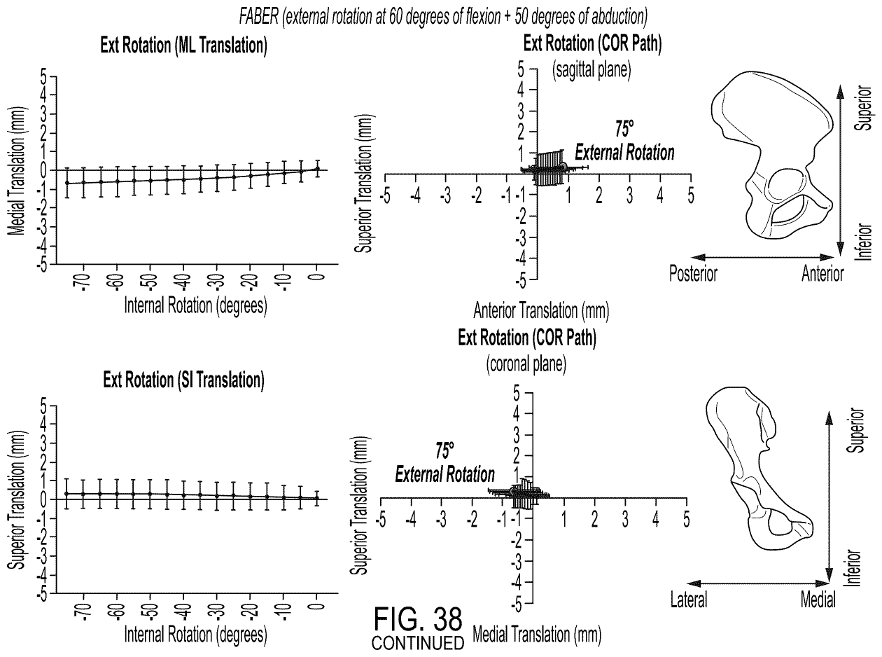

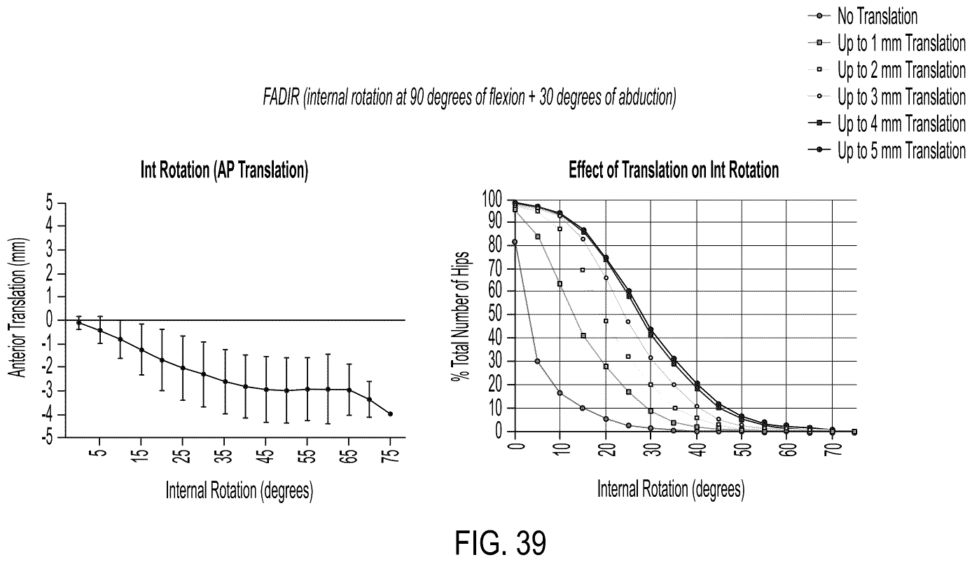

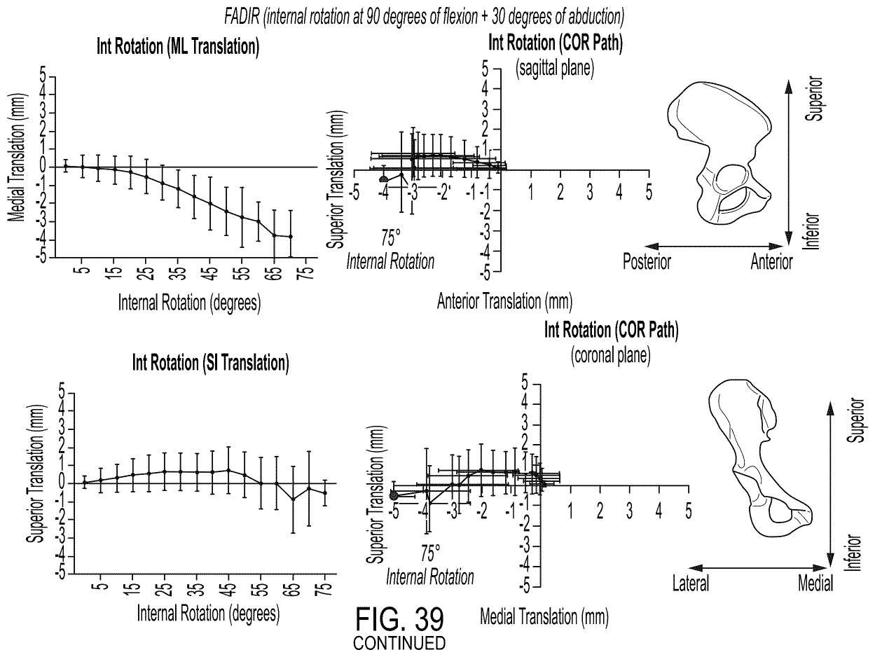

As should be appreciated from the foregoing, the human hip joint is often thought of as a ball and socket joint with a fixed-center of rotation and no translation. This traditional view has been the basis for several simulation platforms to study evaluate hip impingement and instability, in an effort to optimize treatment planning and associated outcomes. There has been a growing number of in vivo imaging and cadaveric studies, suggesting potential hip translations. A recent MRI study have shown an average translation of 2 mm, we up to 7 mm translations in certain positions, among subjects with asymptomatic hips. Such, substantial magnitudes of translation can completely change hip contact and impingement patterns with downstream effects on the treatment plans to reduce impingement or increase hip stability through additional coverage. However, we know little about how hip translation may influence hip impingement. Here we used a validated simulation platform, capable of simulating hip translation during impingement analysis, to study the effect of varying degrees of hip translation (i.e., laxity) on hip impingement risk and impingement free range of motion. Research was conducted to test the theory that an increase in translation would result in increased impingement free range of motion and lower risk of impingement. The results of the research are shown in FIG. 32-40, and the method of the research is shown below.

In a first study, using imaging data from subjects with no documented bone and joint pathology (n=1,222 hips, 611 subjects, age: 30.4±8.8; 67% males) we have mapped the hip COR trajectory under common movements and shown how increased hip translation may influence impingement free range of motion. The effect of COR translation on achieved impingement-free range of motion along with COR trajectories are shown in FIGS. 32-39. Hip flexion was associated with COR posterior (by 4.0±0.9 mm), lateral (only after 100°, by 3.6±1.4 mm), and superior (only after 100°, by 2.0±1.8 mm) translations (P<0.001). Hip extension was only associated with anterior COR translation (by 0.8±0.7 mm, P<0.001). Hip adduction was associated with anterior (by 2.9±1.3 mm), lateral (by 4.0±1.3 mm), and superior (by 4.4±0.6 mm) translations (P<0.01). Hip abduction was only associated with medial (by 2.6±1.5 mm) and inferior (by 2.9±1.7 mm) translations (P<0.02). There were minimal COR translations during hip internal rotations (P>0.05). Hip external rotation was associated with anterior (by 3.7±1.4 mm) and inferior (by 1.2±1.3 mm) translations (P<0.05).

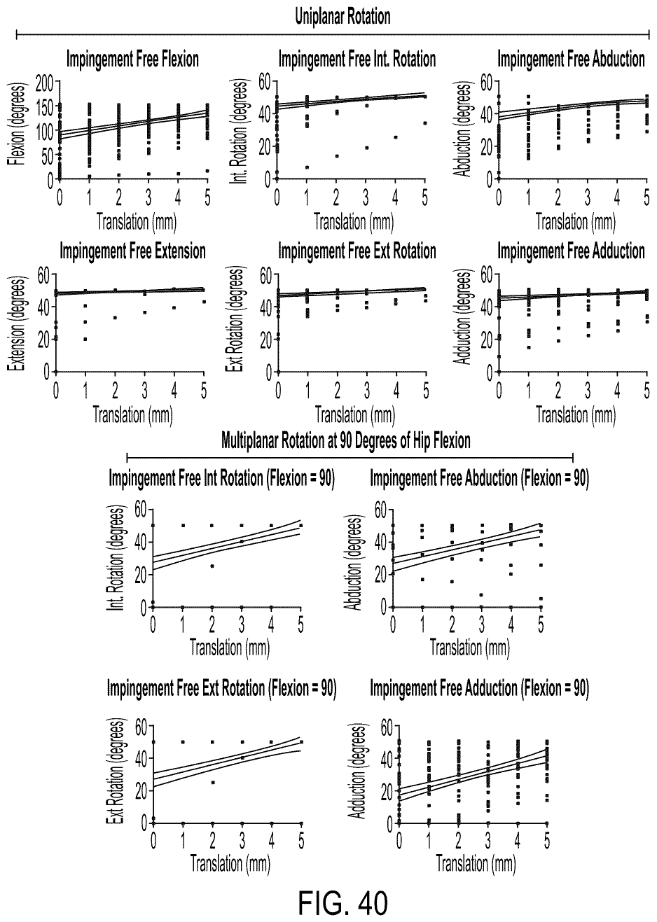

In another study, using imaging data from 51 hips (12 asymptomatic, 18 with FAI, 23 with SCFE) from 39 subjects (age range, 15-47 years old; 42% female) we have shown that during simulated uniplanar rotations, increased translation range from 0 to 5 mm was associated with increased impingement free flexion (R2=0.16, P<. 001), extension (R2=0.04, P<. 001), internal rotation (R2=0.10, P<. 001), external rotation (R2=0.07, P<. 001), abduction (R2=0.11, P<. 001), and adduction (R2-0.04, P<. 001)-FIG. 40. At 90 degrees of hip flexion, increased translation range from 0 to 5 mm was associated with increased impingement free internal rotation (R2=0.12, P<. 001), external rotation (R2=0.12, P<. 001), abduction (R2-0.12, P<. 001), and adduction (R2-0.16, P<. 001)-FIG. 40. Similar trends were observed in subgroup analysis of SCFE and FAI hips. Each mm of hip translation reduced the risk of impingement by 29% under flexion (P<. 001), by 40% under extension (P=. 003), by 58% under internal rotation (at zero flexion, P<. 001), by 44% under external rotation (at zero flexion, P<. 001), by 21% under abduction (at zero flexion, P=. 002), by 34% under adduction (at zero flexion, p<. 001), by 21% under internal rotation (at 90 deg of flexion, P=. 004), by 40% under external rotation (at 90 deg of flexion, P<. 001), by 36% under abduction (at 90 deg of flexion, P<. 001), and by 29% under adduction (at 90 deg of flexion, P<. 001).

Our studies suggest that previously reported hips translations in both asymptomatic and pathologic hips can directly impact hip impingent risk and impingement free range of motion as shown in FIG. 32-40. Considering the fact that hip is not a perfect ball and socket joint with an aspherical femoral head, translation is required to accommodate physiologic rotation (e.g., glide mechanism) without bony impingement between femur and pelvis. The current findings highlight the importance of hips translation when studying hip impingement and casts doubts on the clinical validity of the simulation platforms which rely on fixed center of rotation and do not take into account hip translation. These preliminary observations also highlight the need for comprehensive assessment of normal and pathologic hip translation along with their role in biomechanics, injury risk and response to treatment. Such information can directly impact our treatment approaches, including the choice of conservative (e.g., movement correction through physical therapy and gait training) versus surgical correction of abnormal morphology (e.g., osteotomy). A clear understanding of the role of hip translation in hip function and health will improve clinical care through better diagnosis and personalized treatment planning, which can ultimately lead to improved treatment outcomes, in particular lower risk of osteoarthritis.

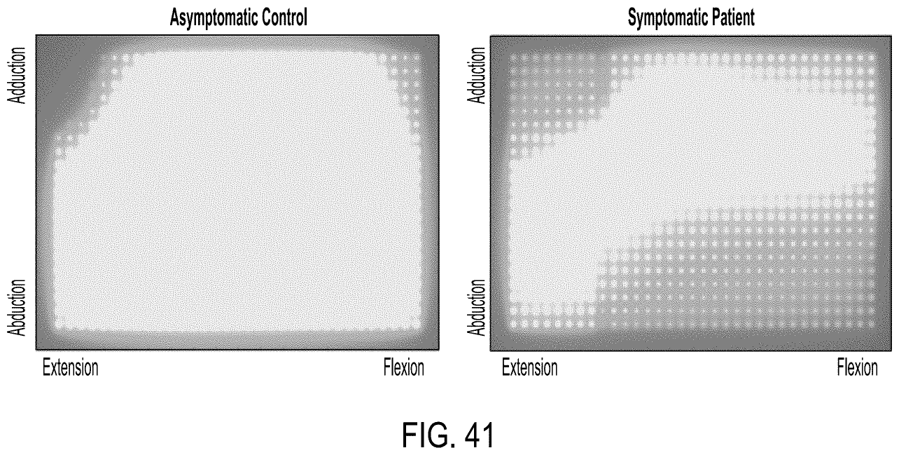

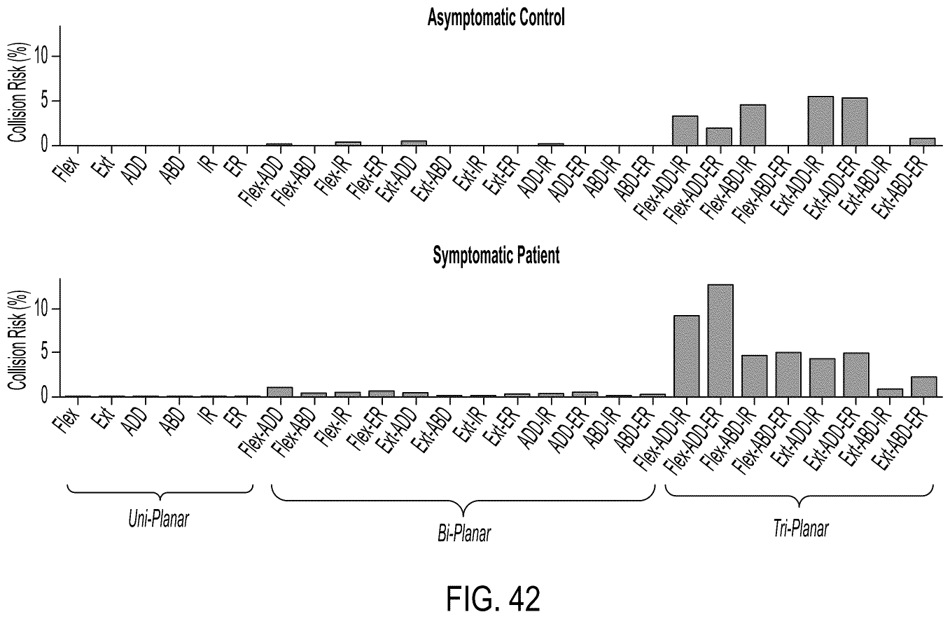

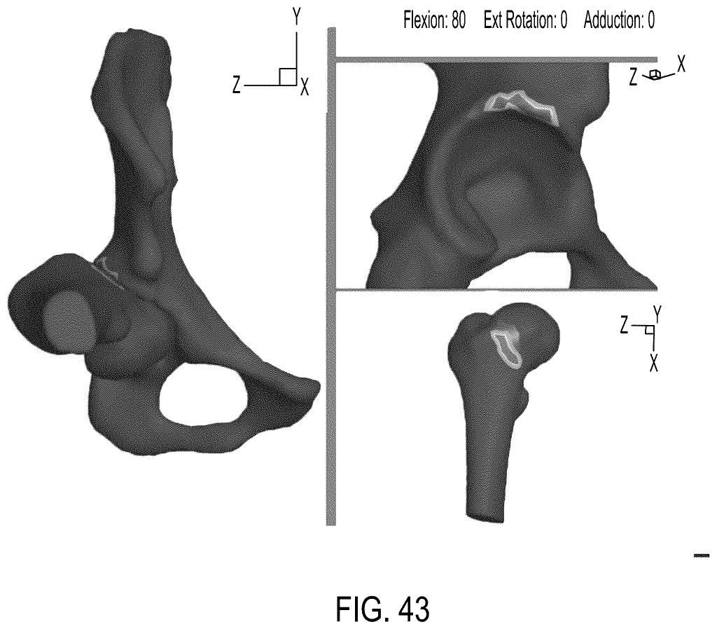

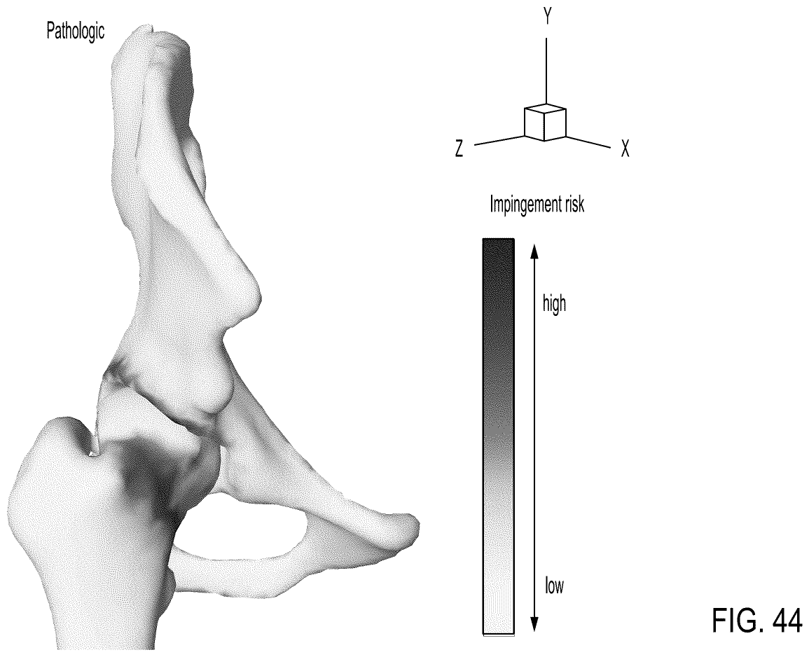

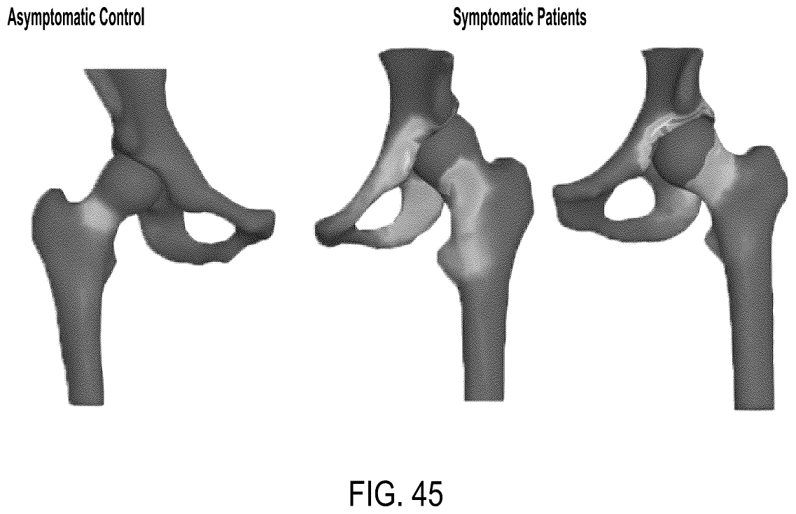

The collision (impingement) detection module result in comprehensive results including ranges of impingement free rotations (FIG. 41), motion related impingement risk (FIG. 42), positional impingement maps (FIGS. 43-44), and global impingement maps (FIG. 45), which show the location and severity of the impingement on the pelvis and femur, across the whole tested range of motion. In oppose to the typical motion specific collision heatmaps, the global heatmap will offer a comprehensive assessment of the overall impingement risk. As shown in the figures, these results can clearly distinguish between normal and pathologic hips, thus facilitating the diagnosis and treatment planning. The results can be also compared to matched historic data to offer tailored assessments to assist diagnosis and treatment planning. Owing to its autonomous nature to process large amount of historic data, VirtualHip has a large body of analyzed cases (tens of thousands) which can be leveraged for adjusted risk assessments and treatment planning for sub populations (e.g., age, sex, race, sports), which is not available in any other existing platforms.

FIG. 41 illustrates density plots indicating the impingement free range of motion (yellow zones) across the tested range of motion. The denser yellow region is indicative of less impingement. As shown in the figure, the asymptomatic hips have a wider impingement free range of motion compared to a symptomatic hip.

FIG. 42 illustrates impingement risk assessments per mode of motion which helps with identifying the motions (activities) leading to more impingement. As shown in the figure, the asymptomatic hips lower risk of impingements compared to a symptomatic hip.

FIGS. 43 and 44 illustrate positional impingement maps which highlight the regions of impingement (red zones) across femur and pelvis for a single simulated position.

FIG. 45 illustrates global impingement maps highlighting the regions of impingement within the tested range of motion. The hotter colors indicated high risk of impingement. As shown in the figure, the asymptomatic hips lower risk of impingements compared to asymptomatic hips.

### Dynamic Femoral Head Coverage Assessment Module.

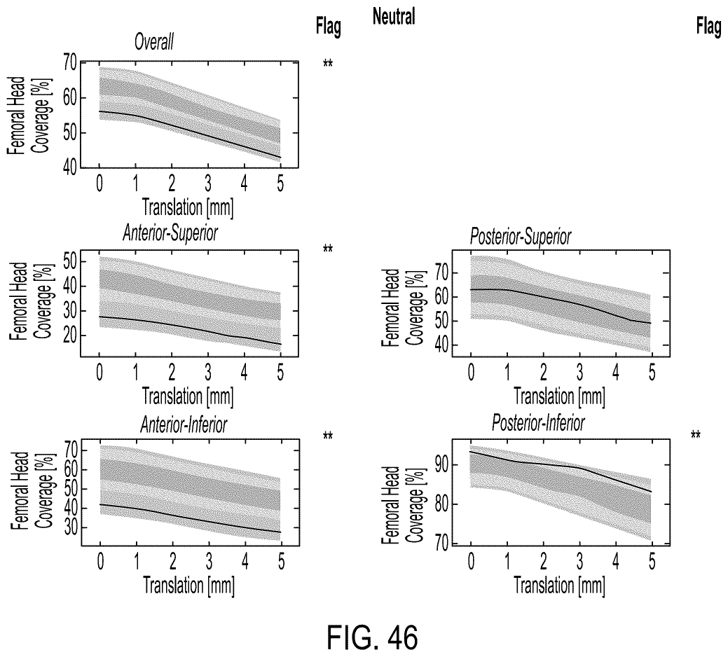

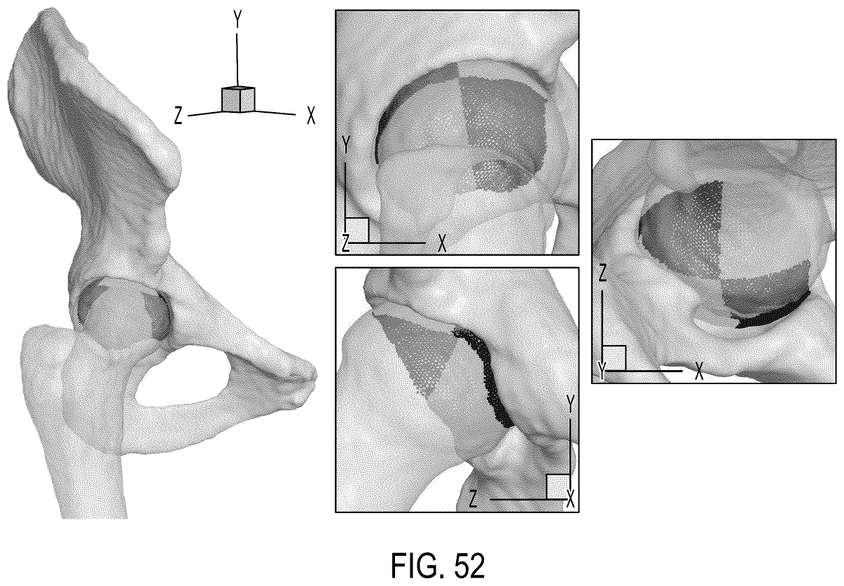

Femoral head coverage (the area of the femoral head covered by acetabulum) is an important surrogate for stability of the hip with shallow coverage leading to instability and potential hip dislocation. On the other hand the over coverage may lead to impingement. As a result, identifying the true coverage across the range of motion is extremely informative for diagnosis and treatment planning. VirtualHip uses ray tracing to identify the elements/regions of the femoral head (automatically identified by the model generation module) covered by the acetabulum automatically identified by the model generation module) across each position. To find the femoral head coverage, i.e., covered area of femoral head by the acetabular surface, for each femoral head element, a ray in the direction of the element normal vector passing from the element center is released. If the ray has intersection with any elements of the acetabular surface, that element of the femoral head is considered as the covered element. Since we already divided the femoral head surface to quadrants, the total covered area as well as covered area of each quadrant is also measured. Similar to the collision detection module, femoral head simulation is based on 6-DOF (3 rotations and 3 translations). In addition to generic range of motion, the patient specific range of motion can be imported by the user from the clinical examination or dynamic ultrasound. process collision detection, since the relative position of femur and pelvis is available in the full range of motion, the femoral head coverage is calculated for all motions. The output of the analysis are areas of coverage and percentage of the areas of coverage both across the whole femoral head and within specific regions (e.g., quadrants). The measurements can also be presented as changes over motion with different translations (FIGS. 46-51) or graphically shown on 3D (FIG. 52). The results can be also compared to matched historic data to offer tailored assessments to assist diagnosis and treatment planning. Owing to its autonomous nature to process large amount of historic data, VirtualHip has a large body of analyzed cases (tens of thousands) which can be leveraged for adjusted risk assessments and treatment planning for sub populations (e.g., age, sex, race, sports), which is not available in any other existing platforms (FIGS. 46-51).

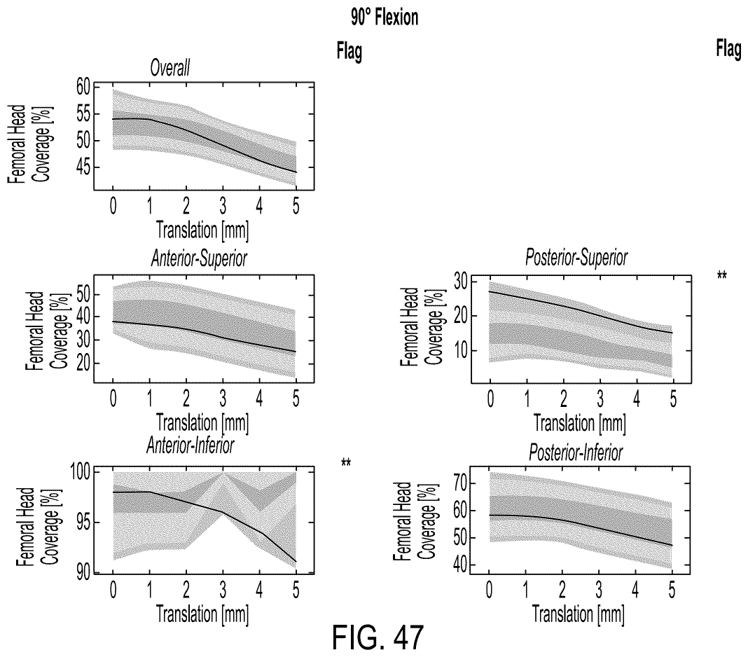

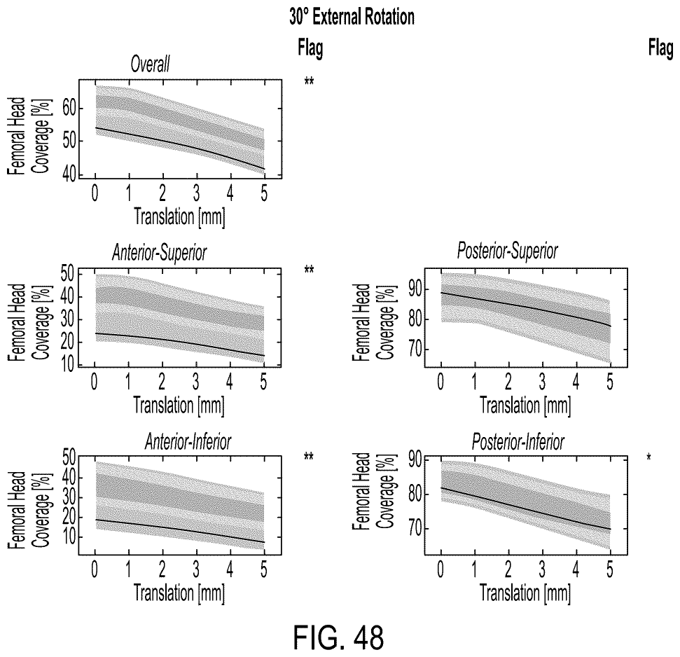

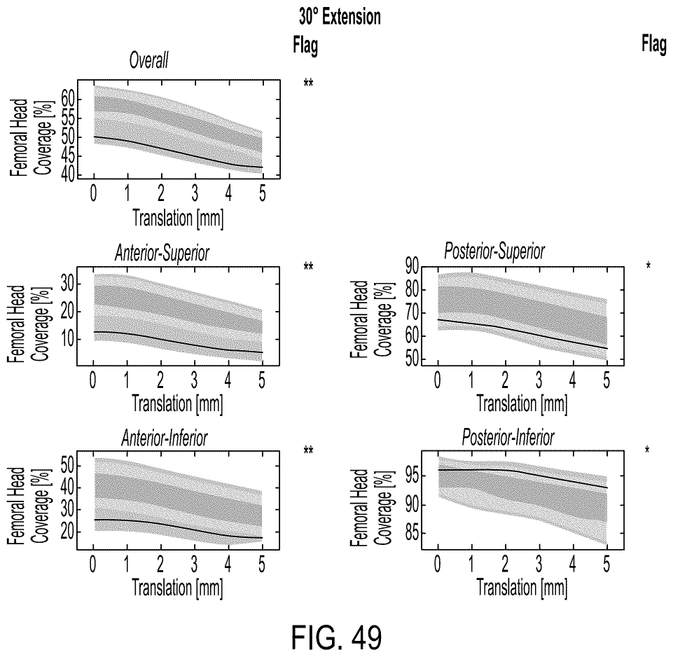

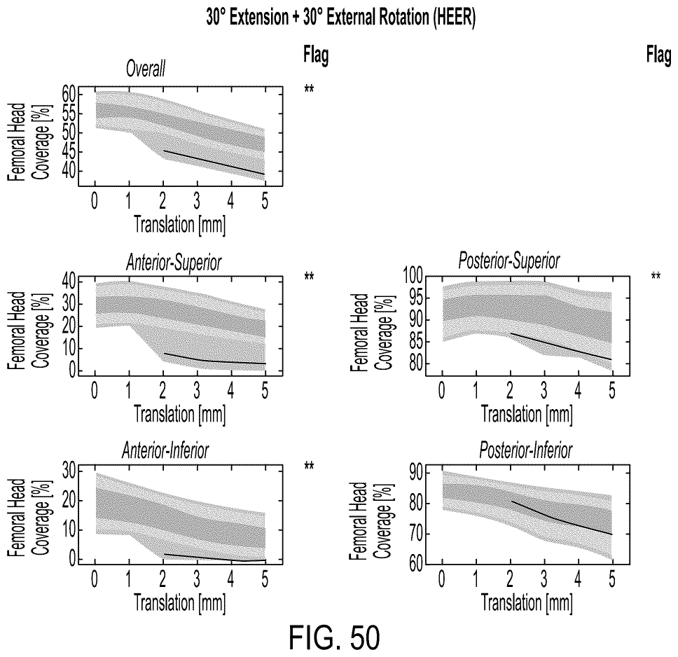

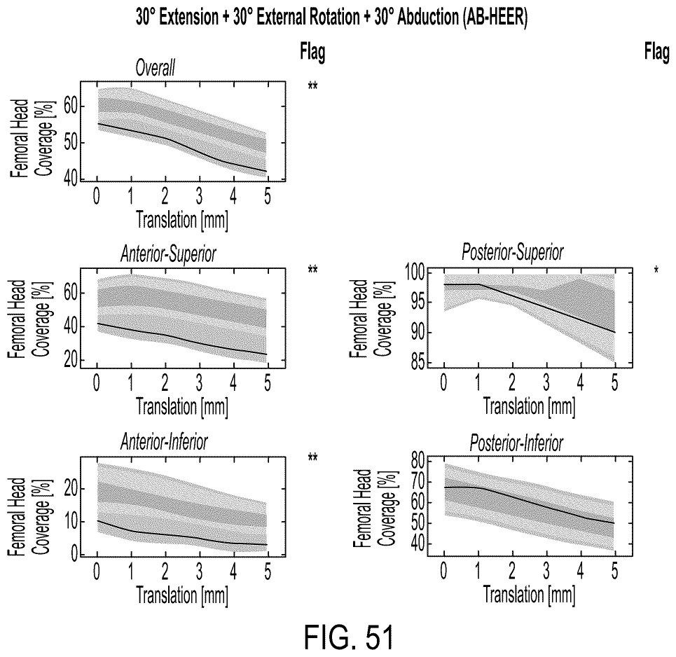

FIGS. 46-52 illustrates changes in femoral head coverage as percentage of overall area during simulated range of motion with different translations and in comparisons to demographically matched controls. FIG. 46 illustrates changes in femoral head coverage as percentage of overall area by increased hip translation during the neutral position compared to demographically matched controls. FIG. 47 illustrates changes in femoral head coverage as percentage of overall area by increased hip translation during hip flexion compared to demographically matched controls. FIG. 48 illustrates changes in femoral head coverage as percentage of overall area by increased hip translation during hip extension compared to demographically matched controls. FIG. 49 illustrates changes in femoral head coverage as percentage of overall area by increased hip translation during hip external rotation compared to demographically matched controls. FIG. 50 illustrates changes in femoral head coverage as percentage of overall area by increased hip translation during hip external rotation and extension compared to demographically matched controls. FIG. 51 illustrates changes in femoral head coverage as percentage of overall area by increased hip translation during hip external rotation+extension and abduction compared to demographically matched controls. In FIGS. 46-51, green is normal range, yellow is borderline abnormal range, and pink is abnormal range.

FIG. 52 illustrates a 3D representation of the regional femoral head coverage during a specific motion. Each color, indicate a region of the quadrants covered by the acetabulum.

### Personalized Treatment Planning Module

Due to the large amount of historic data obtained from analyzing tens of thousands of cases (normal and pathologic hips) and comprehensive assessment of hip anatomy, impingement and coverage, including incorporation of hip translation, VirtualHip is capable of offering guidance for treatment planning. These include suggestions for selecting between conservative (e.g., physical therapy) and surgical treatment, as well as the type of surgery (e.g., osteotomy, osteoplasty), assistance with surgical planning (e.g., the amount of bone removal and fragment reorientation). We use optimization algorithms to develop those solutions based on the clinical input. For example, VirtualHip can identify the amount and direction of the translation needed to move the hip without impingement, based on the comprehensive assessment of impingement under a wide 6-DOF range of motion. If the identified range of translation is deemed achievable (based on historic data and clinical input), the subject can be then considered for conservative treatment (e.g., physical therapy and gait training) to adjust the neuromuscular performance for correcting hip motion to avoid impingement. The same evaluation can be done to minimize hip instability and risk of dislocation by changing the optimization target from impingement to femoral head coverage. In the case, desired coverage can be set by the clinical care team or based on adjusted matched historic data to assess weather translation can be used to achieve the desired coverage during the tested range of motion. VirtualHip can also match the patients to those analyzed and treated before to make recommendations based on historic data. If the analysis (e.g., translation adjustment or matching) or the clinical input was to go ahead with surgical intervention instead of conservative treatment, VirtualHip can use the comprehensive assessments of hip morphology, impingement and coverage to identify the regions required to be trimmed (and the extent of removal) for chondroplasty or osteoplasty, or to predict the amount of rotation for bony fragments on the femur (e.g., derotational osteotomy) or pelvis (e.g., periacetabular osteotomy) to achieve desired outcome (e.g., lower impingement and/or improved coverage). To do such analysis, VirtualHip leverages its large historic database and comprehensive analysis by asking the clinician for input to select the optimization target (e.g., no impingement at a certain position or increase coverage at a certain position). These are significantly important features which can directly influence the success and outcome of hip preservation treatment and are not currently available in a generalizable and personalized manner. We have developed a web-based reporting interface to get the input from the clinical care team to start the process, which will be done on local servers or on the cloud. Once the results are ready the clinicians will be notified to review the results by logging into the web-based interface, where they can see interactive reports for the morphology, impingement, femoral head coverage and treatment planning, including comparison to historic data.

### Comprehensive Reporting to Help a Large Audience

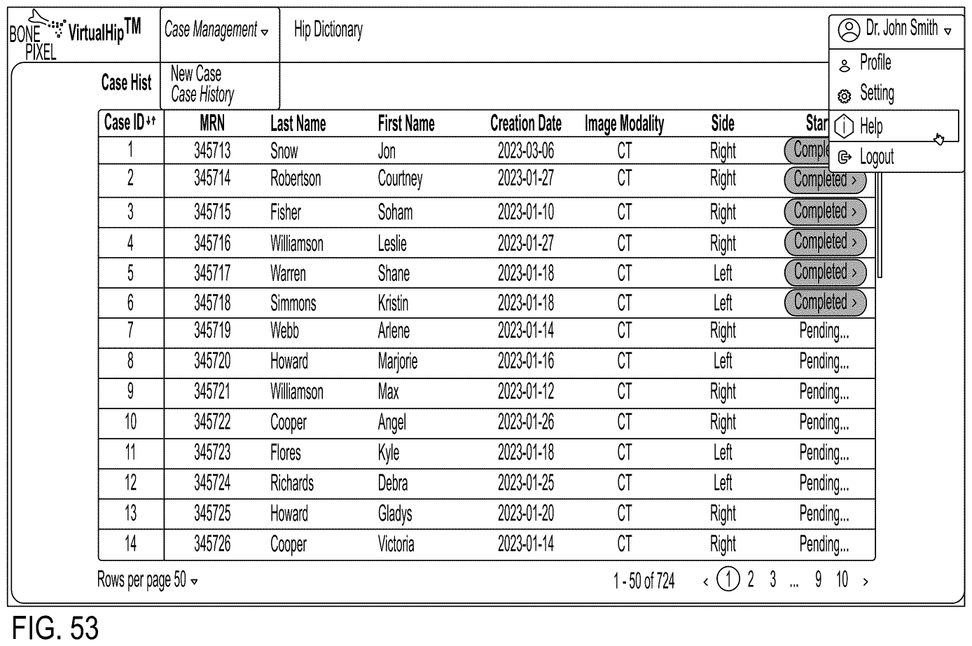

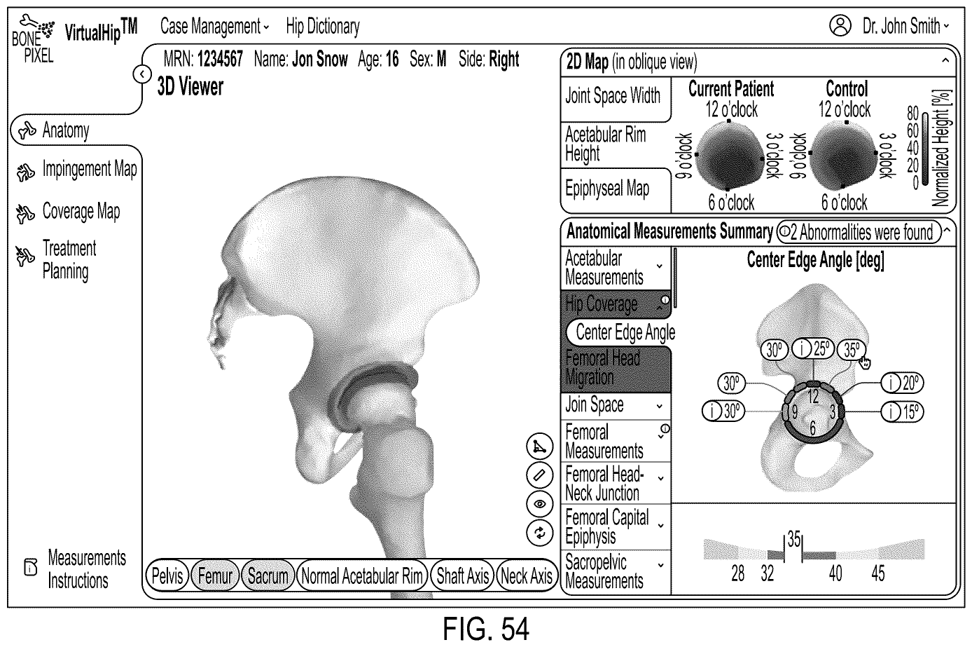

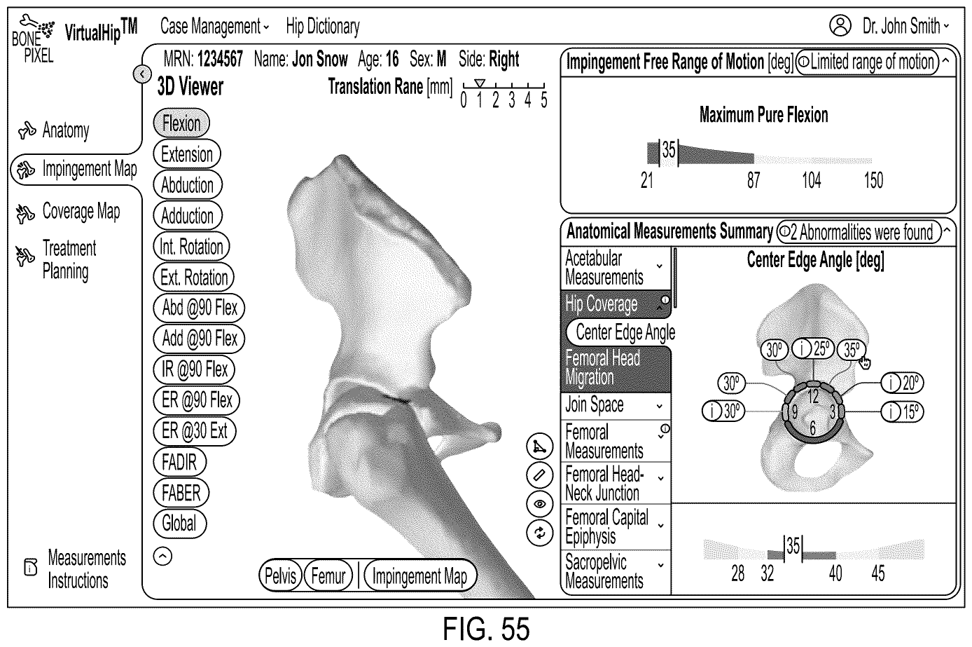

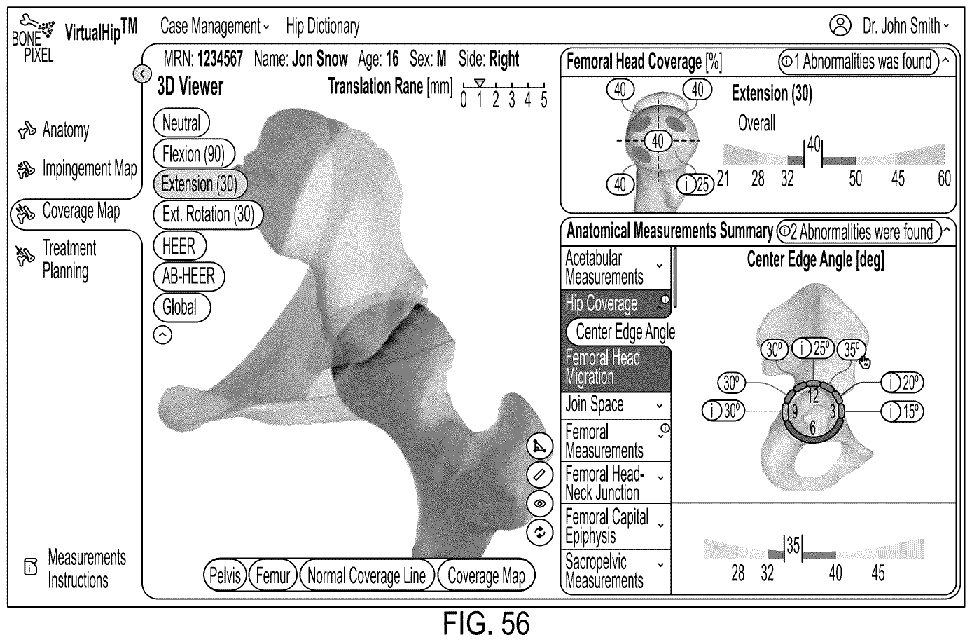

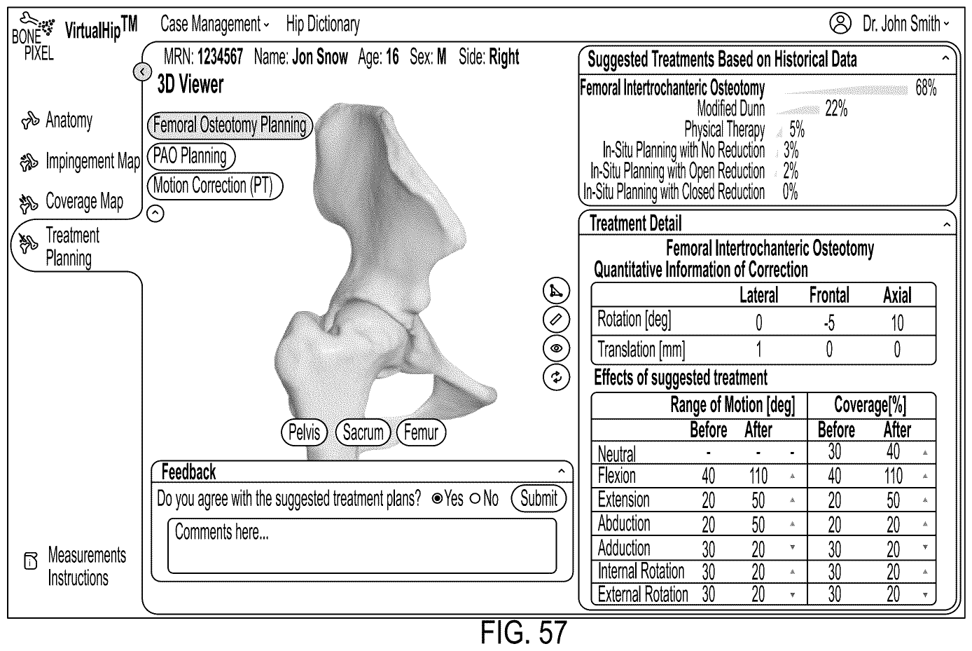

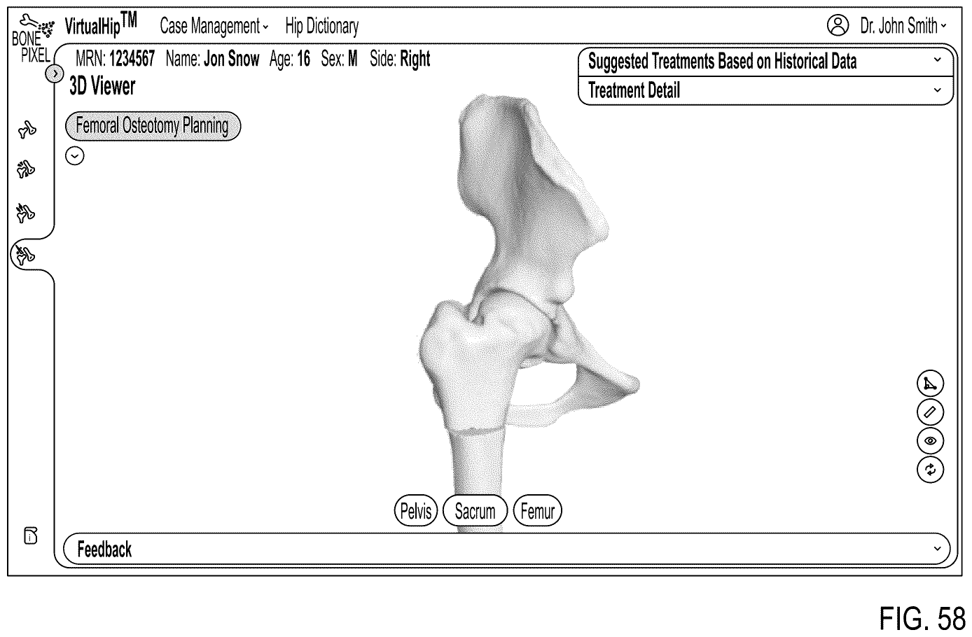

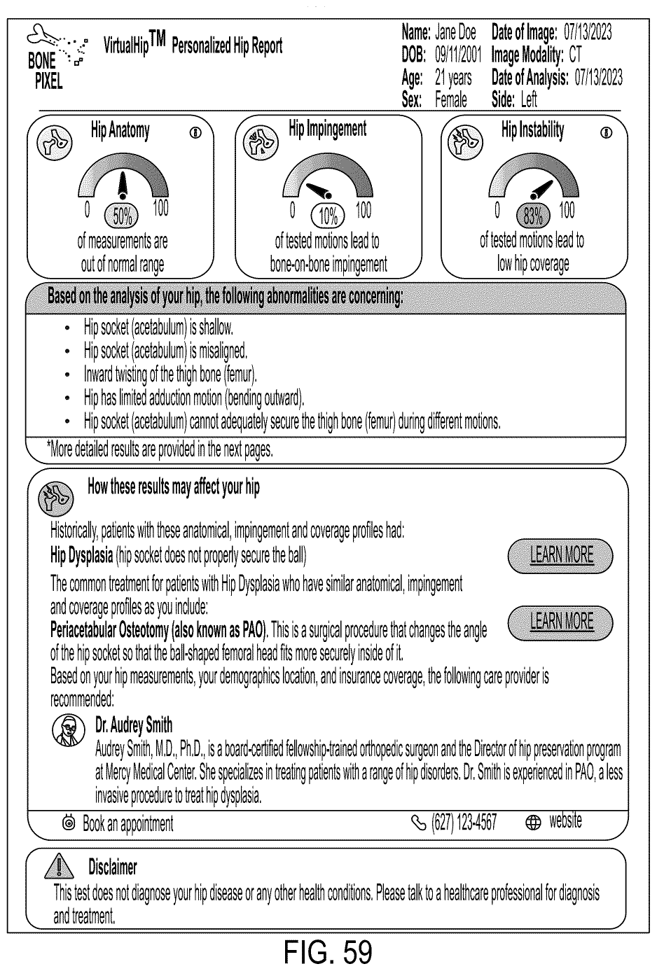

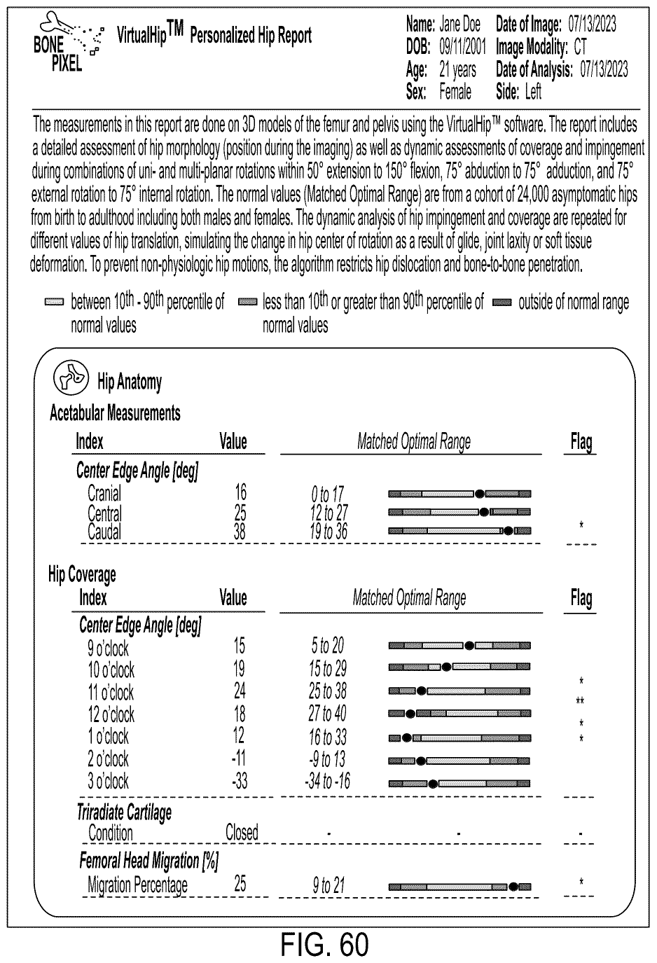

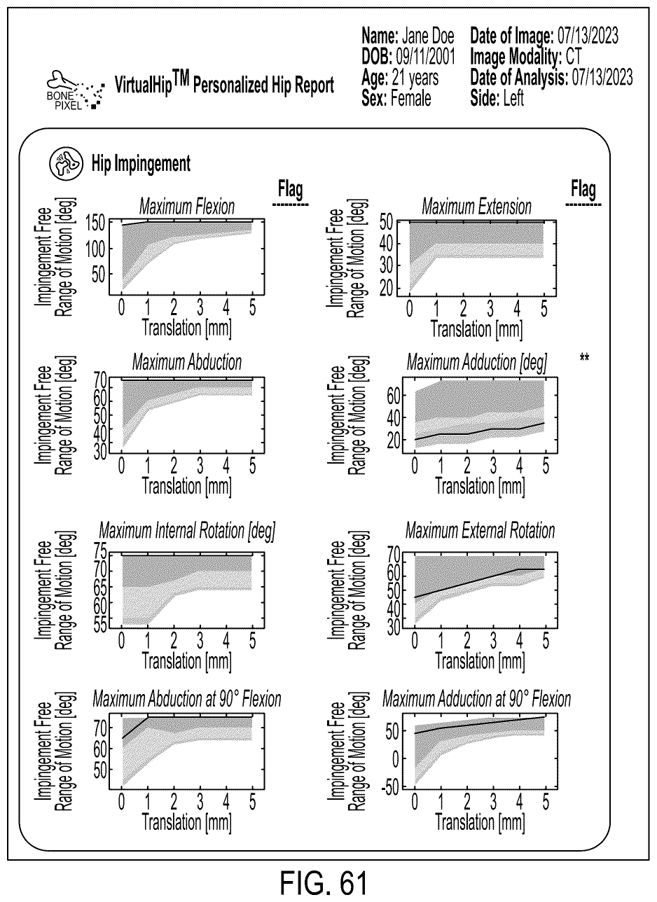

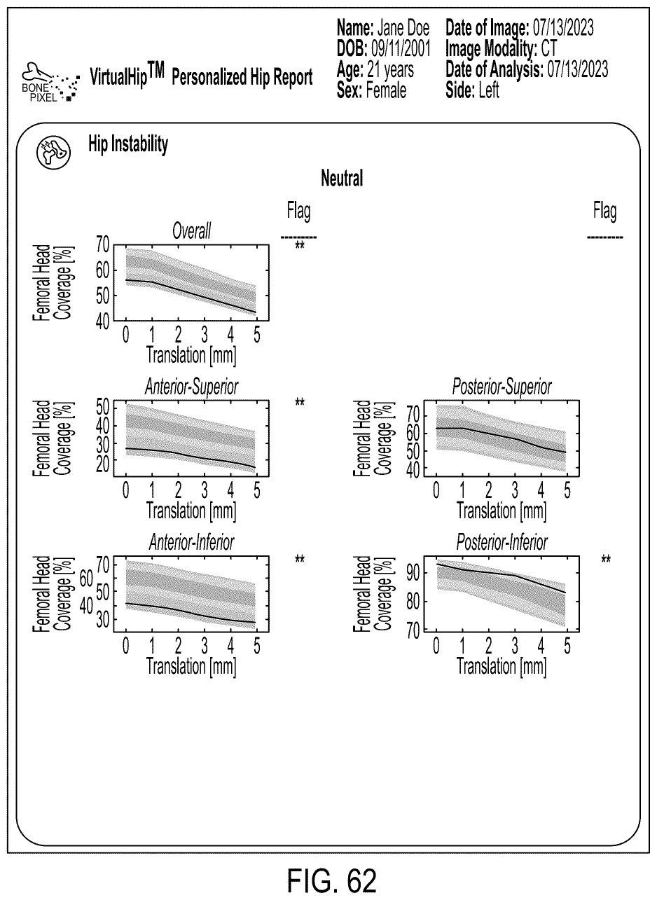

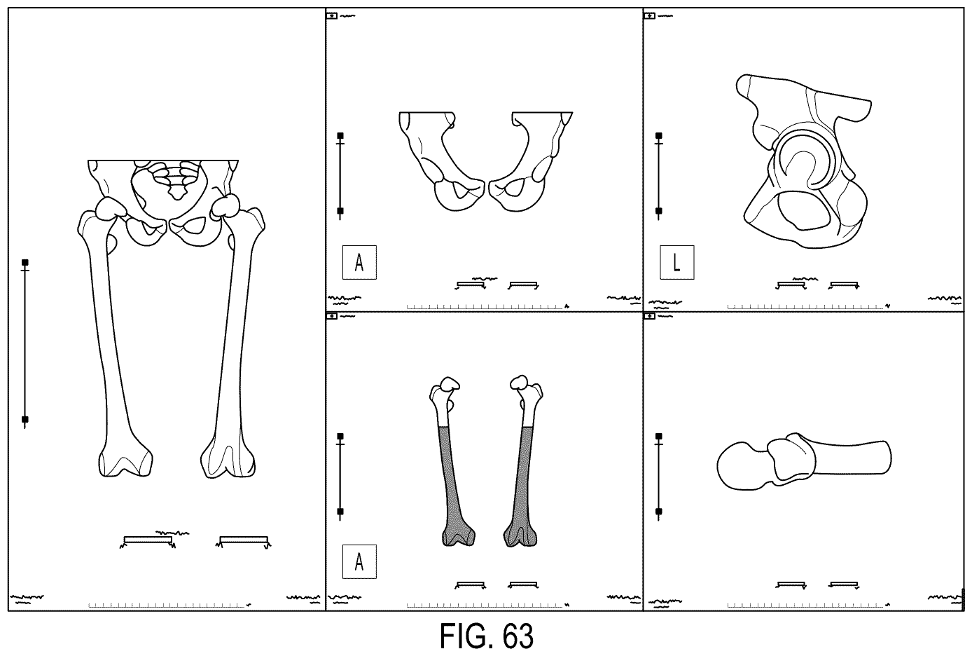

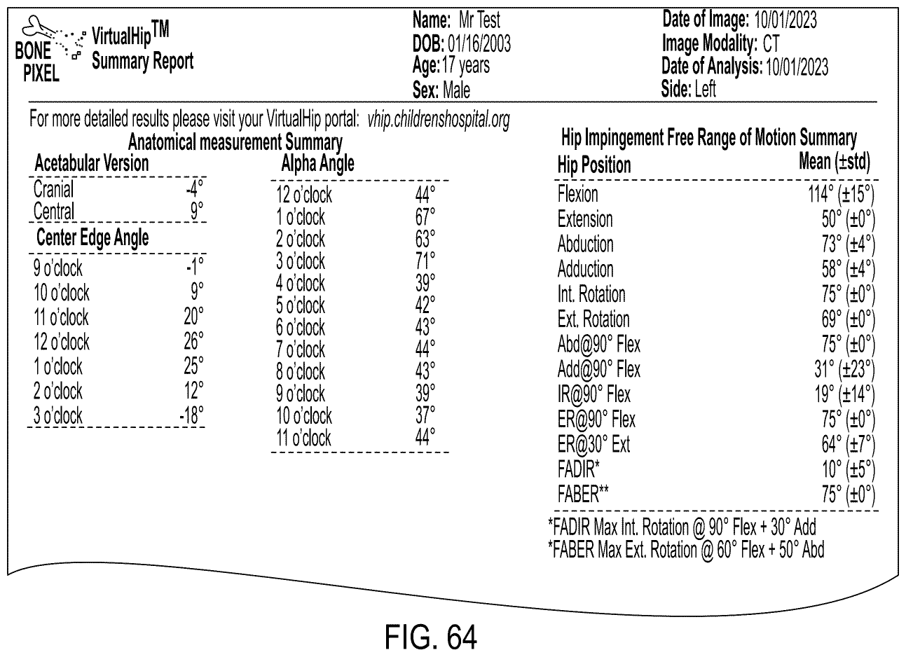

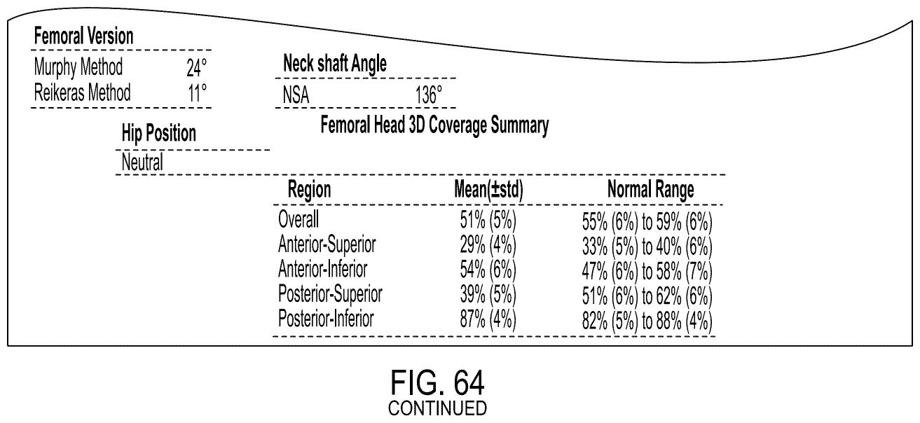

Owing to its unique largescale database and automated pipeline, VirtualHip generates a set of comprehensive and easy to use reports including interactive web based reporting portal (FIGS. 53-58) and static reports (e.g., PDF) (FIGS. 59-64) which can be stored within the electronic health records (EHR) for a wide range of audience including patients, surgeons and radiologists. These reports can include 3D models of the joint, including its tissues, quantitative information on joint morphology and dynamic function (e.g., impingement and instability) along with demographically matched growth charts (percentiles), and personalized risk assessments and treatment suggestions.

FIGS. 53-64 illustrate examples of user interfaces for VirtualHip, with which some implementations of the platform may operate.

Additional Research into Hip Translation

As should be appreciated from the foregoing, the human hip joint is often thought about as a ball and socket joint with a fixed-center of rotation and no translation. This traditional view has been the basis for several simulation platforms to study evaluate hip impingement and instability, in an effort to optimize treatment planning and associated outcomes. There has been a growing number of in vivo imaging and cadaveric studies, suggesting potential hip translations. However, majority of those studies are in pathologic hips and/or under limited ranges of motion. Thus, very little is known on magnitude of those translations under single- and multi-planar physiologic rotations and in normal (asymptomatic) hips. Further, it is unclear if those translations are influenced by the extent of hip rotation as well as hip morphology. The presence of translation and coupled motion can completely change hip contact and impingement patterns. Despite this importance, the modern medical system knows little about how hip translation and rotation are related in normal and pathologic hips.

A research study was conducted to determine whether the femoral head exhibits significant translations in asymptomatic hips, whether femoral head translations correlate to femoral rotations, and whether a range of femoral head translations correlate to hip morphology. A cross-sectional study was conducted in which eleven subjects (23-47 years; 64% female) with asymptomatic hips underwent hip MRI under a range of physiologic positions (10 positions per subject). MRIs were used to generate 3D hip models to quantify femoral six-degrees of freedom motion of the femur (i.e., 3 rotations and 3 translations). Femoral head diameter, acetabular diameter, lateral center edge angle (LCEA), alpha angle, femoral anteversion, acetabular version and inclination, and neck-shaft angle were measured from MRI.

The study showed that femoral head translated anteriorly by 2±1 mm (maximum 5 mm, P<0.001), posteriorly by 1±1 mm (maximum 6 mm, P<0.001), superiorly by 2±2 mm (maximum 7 mm, P<0.001), inferiorly by 2±2 mm (maximum 6 mm, P<0.001), laterally by 1±1 mm (maximum 4 mm, P<0.001), and medially by 2±1 mm (maximum 5 mm, P<0.001). Femoral flexion was associated with posterior translation of the femoral head (P=. 038). Femoral abduction was associated with medial translation of the femoral head (P=. 042). Higher femoral anteversion and smaller alpha angle were associated with higher range of femoral head translation in the anterior-posterior direction (P<. 04). Smaller femoral anteversion, higher acetabular inclination, smaller lateral center edge angle, and lower neck-shaft angle were associated with higher range of femoral head translation in the superior-inferior direction (P≤0.03).

These findings support femoral head translation of up to 7 mm during physiologic ranges of rotations in normal asymptomatic hips, which are potentially related to hip rotations and morphology. These translations can significantly influence femur's articulation inside the acetabulum, with direct impact on hip stability and impingement. Further investigations are warranted to understand the normal and pathologic hip translation and their impact on hip health.

The study confirms the presence of hip translation in asymptomatic hips. Although, on average, these translations are within 1-2 mm, the study revealed instances where asymptomatic hips can translate up to 7 mm. Such substantial magnitude of translation cast doubt on the predictions of simulations platforms based on fixed hip center of rotation and no translation. Those translations may also affect the treatment plans to reduce impingement or increase hip stability through additional coverage. Moreover, the findings, highlight potential relationships between certain hip rotations and translations, as well as between hip morphology and translation. Overall, the current study confirms the existence of hip translation in asymptomatic hips, and highlight the need for further largescale efforts to study normal and pathologic hip translations and the factors influencing them (e.g., hip rotation and morphology).

### Further Discussion

While the hip is conventionally thought of as a ball and socket joint with purely rotational motion around a fixed center, a growing number of studies have shown significant femoral head translation in both cadaveric models and in vivo. Previous cadaveric analyses suggested that damage to soft tissue components of the acetabulum increases femoral head translation. Several in vivo studies analyzed femoral head translation and rotation in specific athletic movements to investigate their influence on the development of hip disease. Femoracetabular impingement syndrome (FAI) is of particular interest to this new hip joint theory because of FAI's association with early onset hip pain and secondary osteoarthritis in athletically active young adults. Extensive biomechanical studies were conducted to investigate the physiologic movements that cause impingement symptoms amongst these hip morphologies, revealing repetitive low impact loading during flexion and internal rotation as a primary cause. Elucidating the translational and rotational profile of the femoral head within the acetabular socket during these physiological movements would provide new insights regarding the etiology of FAI and other hip diseases.

While investigations examining the biomechanical profile of the hip joint grow in number, methods for analyzing hip translation vary significantly across the literature. Techniques include dynamic fluoroscopy, motion capture devices, musculoskeletal models, dynamic ultrasound, roentgen stereophotogrammetric analysis, plain x-ray analysis, and 3D segmentation from computed tomography (CT), and magnetic resonance imaging (MRI). Many studies have utilized these tools to compare diseased hips to native controls. But, no study to date has established how in-vivo hip translation and rotation manifest in asymptomatic native hips undergoing a variety of physiologic movements.

This research assessed detailed motion of the hip joint in six-degrees of freedom across a range of physiologically relevant movements using MRI and three-dimensional analysis in a cohort of volunteers with asymptomatic hips. The study hypothesized that: 1) femoral head exhibits significant translations in asymptomatic hips, 2) femoral head translations correlate to femoral rotations, and 3) range of femoral head translations correlate to hip morphology.

### Methods

### Subjects

Following IRB approval, 11 healthy volunteers with asymptomatic hips were recruited to participate in this study. Subjects included if they were 18-50 years old with a body mass index of lower or equal to 30 Kg/m². Subjects were excluded if they were pregnant or had a history of growth-related disorders, diseases of bone and connective tissue, neuromuscular diseases, prior hip surgery, any history of hip degeneration and arthritis, and self-reported restricted joint range of motion of the hip and knee. Eligible subjects were consented prior to participating in the study.

### MR Imaging

Following enrollment, subjects underwent MR imaging using a coronal T1-weighted VIBE Dixon sequence which was optimized to have a large field of view (FoV), high spatial resolution and short acquisition time (repetition time/echo time [TR/TE]=4/1.23, 420×420 FOV, 1.1-mm slice thickness, 320×256 matrix). The imaging was done in a Siemens 3T Skyra scanner using a 18-channel flex body coil. Dixon techniques present many advantages compared with other fat suppression techniques including: 1) the robustness of fat signal suppression, 2) the possibility to combine these techniques with all types of sequences (gradient echo, spin echo) and different weightings (T1-, T2-, proton density-, intermediate-weighted sequences), and 3) the availability of images both with and without fat suppression from one single acquisition. The generation of these sequences are helpful to accurately delineate the boundaries of the anatomical structures during segmentation.

Subjects were scanned under 10 positions (Table 1). All the positions were passive and guided by a trained member of the team. Once the limbs were in position, they were fixed by foam wedges and straps to avoid motion artifacts. The neutral scan covered the complete femurs and pelvis, while the other scans solely covered the pelvis and proximal femurs. The average scan time, including positioning, was 52±11 minutes. MR images were reviewed by an experienced fellowship trained pediatric musculoskeletal radiologist, to evaluate all subjects for occult hip pathology.

TABLE 1
Studied positions.

| | Body | Studied Leg | Studied Hip | Contralateral | Contralateral |
|---|---|---|---|---|---|
| Position | Position | Position | Position | Leg Position | Hip Position |
| Neutral | supine | straight | neutral | straight | neutral |
| Mid Flexion | supine | flexed | flexed (~30°) | straight | neutral |
| Max Flexion | supine | flexed | flexed ^{a} | straight | neutral |
| Internal Rotation | supine | straight | internally rotated ^{a} | straight | neutral |
| Internal Rotation + | supine | flexed | flexed (~30°) + | straight | neutral |
| Mid Flexion | | | internally rotated ^{a} | | |
| Internal Rotation + | supine | flexed | flexed ^{a} + internally | straight | neutral |
| Max Flexion | | | rotated ^{a} | | |
| Adduction | Supine | straight ^{b} | adducted ^{a} | straight | neutral |
| FABER ^{c} | supine | flexed | FABER (mimicking | straight | neutral |
| | | | the clinical exam) | | |
| Extension | quadruped | straight | extended ^{a} | flexed | flexed |
| Lateral Abduction | lateral | straight | abducted ^{a} | straight | neutral |
| | decubitus | | | | |

^{a} To the maximum possible level based on patient comfort and ability to fit inside the magnet
^{b} Crossing over the contralateral leg
^{c} Flexion Abduction External Rotation

### Image Processing

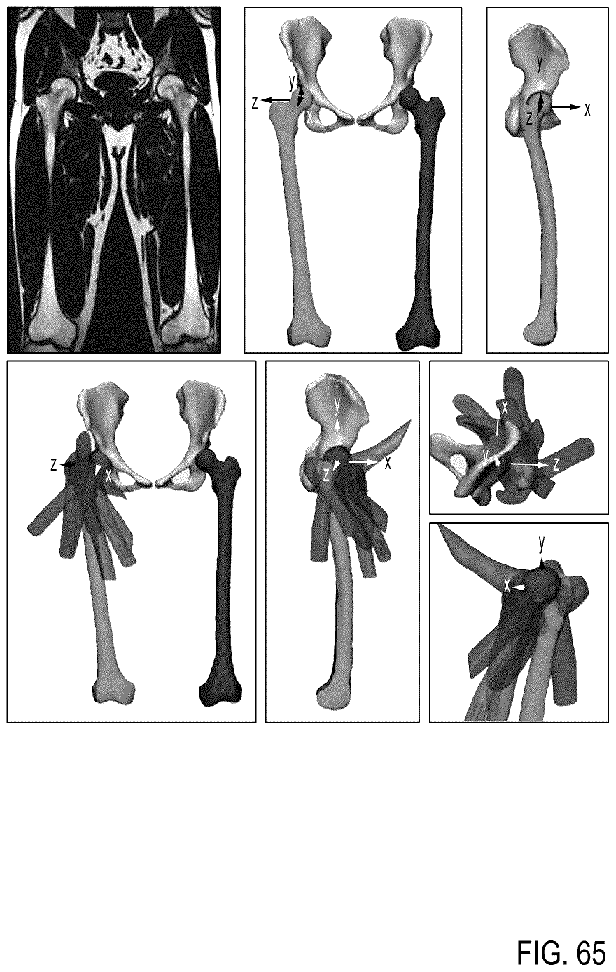

An orthopedic surgeon manually segmented the pelvis and femurs for all scans and patients using a commercial image processing software (Mimics, Materialise, Belgium). The segmentations were then reviewed by two independent investigators, as shown in FIG. 65. The segmented masks were then used to reconstruct 3D geometries for each bone (FIG. 65). The reconstructed geometries, were then imported to 3-matic software (Materialise, Belgium) to conduct the measurements. The reconstructed pelvis and femurs from the neutral scan were translated into an anatomical coordinate system based on the International Society of Biomechanics (ISB) recommended coordinate system. A best-fit sphere was used to find the center of the femoral head which was used as the origin of the femur coordinate system (FIG. 65). For each subject, the reconstructed 3D models of the hip from all other motions were then superimposed on the neutral models by registering the pelvises to the neutral pelvis using a N-point registration algorithm (FIG. 65). The superimposed femurs were then used to measure femoral translations (in mm) and rotations (in degrees) relative to the neutral femur. To further assess the extent of femoral head translation independent of patient size, we normalized the translations to the diameter of the acetabulum, measured in 3D using the best-fit sphere, and reported them as percentage of acetabulum diameter. Neutral MRIs were also used to measure femoral anteversion, neck-shaft angle, alpha angle, lateral center edge angle and acetabular inclination following established techniques. Segmentation and analysis were performed on deidentified MRIs to minimize bias.

More particularly, FIG. 65 illustrates (top row) Development of 3D models from MRI and definition of anatomical coordinate system (+X: Anterior, +Y: Superior, +Z: Lateral). FIG. 65 also illustrates (bottom row) Model registration to calculate the relative rotation and translation of the femur with respect to the neutral position. The transparent femora are the segmented femora from the different positions, which have been registered to the neutral position by matching the pelvis models.

### Statistical Analysis

Descriptive statistics and graphs were used to characterize the femoral head translation. To test our first hypothesis, the average femoral head translations in each direction (i.e., anterior, posterior, inferior, superior, medial, and lateral) across all tested positions were calculated and tested to see if they are significantly different from 0 (no translation) using t-tests. To test the second hypothesis, bivariate linear regression was used to assess the associations between translation (continuous dependent variable) and rotation (continuous independent variable). Separate analysis was done for each pair of translations and uni-planar rotations (e.g., anteroposterior translation vs flexion-extension). Next a multivariate linear regression was used to assess the relationships between translation (continuous dependent variable) and multi-planar rotation (continuous independent variables). For this analysis, the all three rotations (i.e., flexion-extension, abduction-adduction, and internal-external rotations) were entered into the model as independent variables. The rotations with a p-value>0.1 were eliminated using a backward stepwise procedure. To test the second hypothesis, all the rotations and translations for all the positions were pooled (n=99). To test the third hypothesis, Pearson correlation was used to assess the relationships between hip anatomy and range of femoral head translations. For this analysis, the range of translation for each direction was calculated (e.g., anteroposterior translation range was calculated as maximum anterior translation-maximum posterior translation across all tested positions for each subject). This led to a sample size of 11 per Pearson correlation tests. All P-values were two sided and considered statistically significant at α=0.05. The analyses were done using SPSS (v27, IBM, Armonk, NY, USA).

### Results

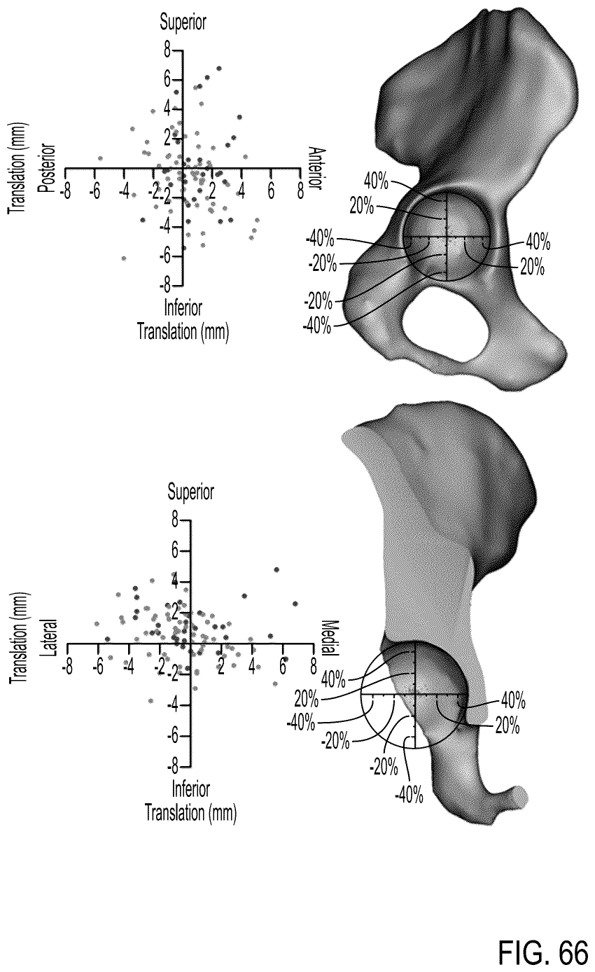

Out of 11 recruited subjects, there were 4 males and 7 females. The baseline characteristics and anatomical indices for the subjects are presented in Table 2. The achieved range of rotations and translations across all subjects are presented in Table 3. On average, femoral head translated anteriorly by 2±1 mm, posteriorly by 1±1 mm, superiorly by 2±2 mm, inferiorly by 2±2 mm, laterally by 1±1 mm, and medially by 2±1 mm. These translations were all statistically significant compared to no translation (0 mm; P<0.001 for all comparisons). The distribution of the femoral head translation in the sagittal and coronal planes are presented in FIG. 66.

TABLE 2
The baseline characteristics and anatomical indices

| Index | Mean ± SD | Range |
|---|---|---|
| Age | 30 ± 7 | 23-47 |
| Mass (Kg) | 70.5 ± 14.1 | 54.4-92.7 |
| Height (m) | 1.7 ± 0.1 | 1.5-1.9 |
| BMI (Kg/m²) | 24.8 ± 2.4 | 20.8-28.5 |
| Femoral Head Diameter (mm) | 44 ± 5 | 37-51 |
| Acetabular Diameter (mm) | 54 ± 5 | 48-62 |
| Femoral Anteversion (degrees) | 7.8 ± 7.1 | −3.5-17.6 |
| Acetabular Version (degrees) | 19.4 ± 6.7 | 9.5-27.1 |
| Acetabular Inclination (degrees) | 48.1 ± 4.4 | 39.3-56.9 |
| Lateral Center Edge Angle (degrees) | 33.3 ± 5.6 | 26.5-45.8 |
| Neck-Shaft Angle (degrees) | 135.1 ± 3.8 | 129.8-141.2 |
| Alpha Angle (degrees) | 46.5 ± 12.8 | 28.9-69.0 |

TABLE 3
Achieved range of motion across all the subjects.
The femoral head translations are presented in
mm and as percentage of acetabulum diameter.

| Motion | Range |
|---|---|
| Extension-Flexion | Up to 25 degrees (Extension)- |
| | Up to 112 degrees (Flexion) |
| Adduction-Abduction | Up to 55 degrees (Adduction)- |
| | Up to 53 degrees (Abduction) |
| External Rotation - | Up to 56 (External Rotation)- |
| Internal Rotation | Up to 52 degrees (internal rotation) |
| Posterior- | Up to 6 mm or 11% (posterior)- |
| Anterior Translation | up to 5 mm or 10% (anterior) |
| Inferior- | Up to 6 mm or 12% (inferior)- |
| Superior Translation | up to 7 mm or 11% (superior) |
| Medial- | Up to 5 mm or 9% (medial)- |
| Lateral Translation | up to 4 mm or 7% (lateral) |

FIG. 66 illustrates the distribution of the femoral head translation in the sagittal (top row) and coronal (bottom row) planes. The center of the coordinate system is located at the center of the femoral head. The blue dots are males and red dots are females. The normalized translations (% of acetabular diameter) are presented inside the acetabulum model.

The bivariate regression coefficients for associations between femoral rotations and translations are presented in Table 4. Among all tested associations, only those between femoral flexion-extension and anterior-posterior translation, and between abduction-adduction and lateral-medial translations were statistically significant. In general, femoral flexion was associated with posterior translation of the femoral head (P=0.038). Similarly, femoral abduction was associated with medial translation of the femoral head (P=0.042). The multivariate analysis resulted in the same findings, with no additional rotational predictors remaining included in the models.

TABLE 4
Regression coefficient (β [95% CI], R²) for
associations between femoral rotations and femoral
head translations. The significant correlations are bold.

| | Posterior (−)- | Inferior (−)- | Medial (−)- |
|---|---|---|---|
| | Anterior (+) | Superior (+) | Lateral (+) |
| | Translation | Translation | Translation |
| Extension (−), | β = −0.02 | β = 0.004 | β = −0.002 |
| Flexion (+) | (−0.03-0.00) | (−0.02-0.02) | (−0.01-0.01) |
| | R² = 0.04 | R² = 0.00 | R² = 0.004 |
| | P = .038 | P = .702 | P = .724 |
| Adduction (−), | β = −0.02 | β = −0.01 | β = −0.02 |
| Abduction (+) | (−0.01-0.04) | (−0.04-0.02) | (−0.04-0.00) |
| | R² = 0.02 | R² = 0.00 | R² = 0.04 |
| | P = .145 | P = .541 | P = .042 |
| External | β = 0.01 | β = −0.003 | β = −0.01 |
| Rotation (−), | (−0.01-0.02) | (−0.01-0.02) | (−0.02-0.01) |
| Internal | R² = 0.01 | R² = 0.01 | R² = 0.01 |
| Rotation (+) | P = .303 | P = .750 | P = .353 |

The correlation coefficients for associations between range of femoral head translations and hip anatomy are presented in Table 5. In general, higher femoral anteversion and smaller alpha angle were associated with higher range of femoral head translation in the anterior-posterior direction (P<0.04). Smaller femoral anteversion, higher acetabular inclination, smaller lateral center edge angle, and lower neck-shaft angle were associated with higher range of femoral head translation in the superior-inferior direction (P≤0.03). There were no significant associations between quantified hip anatomical features and range of femoral head translation in the lateral-medial direction (P>0.1).

TABLE 5
Pearson correlation coefficient (r [95% CI]) for
associations between femoral head range of translations
and hip anatomy. The significant correlations are bold.

| | Posterior- | Inferior- | Medial- |
|---|---|---|---|
| | Anterior | Superior | Lateral |
| | Translation | Translation | Translation |
| | Range | Range | Range |
| Femoral Head | r = −0.41 | r = −0.05 | r = −0.11 |
| Diameter | (−0.81-0.25) | (−0.63-0.56) | (−0.67-0.52) |
| | P = .212 | P = 877 | P = .748 |
| Acetabular | r = −0.51 | r = −0.10 | r = 0.02 |
| Diameter | (−0.85-0.13) | (−0.66-0.53) | (−0.59-0.61) |
| | P = .107 | P = .769 | P = .954 |
| Femoral | r = 0.67 | r = −0.74 | r = −0.51 |
| Anteversion | (0.12-0.91) | (−0.93-−0.26) | (−0.85-0.12) |
| | P = .024 | P = .009 | P = .105 |
| Acetabular | r = 0.25 | r = −0.07 | r = −0.36 |
| Version | (−0.41-0.74) | (−0.64-0.55) | (−0.31-0.79) |
| | P = .459 | P = 0.830 | P = .278 |
| Acetabular | r = −0.18 | r = 0.66 | r = 0.14 |
| Inclination | (−0.70-0.47) | (0.09-0.90) | (−0.49-0.69) |
| | P = .594 | P = .028 | P = .672 |
| Lateral Center | r = −0.22 | r = −0.67 | r = −0.43 |
| Edge Angle | (−0.73-0.43) | (−0.91-−0.12) | (−0.82-0.23) |
| | P = .509 | P = .024 | P = .188 |
| Neck-Shaft | r = −0.06 | r = −0.65 | r = −0.26 |
| Angle | (−0.63-0.56) | (−0.89-−0.08) | (−0.74-0.40) |
| | P = .869 | P = .030 | P = .437 |
| Alpha Angle | r = −0.64 | r = −0.14 | r = −0.22 |
| | (−0.89-0.06) | (−0.68-0.50) | (−0.72-0.44) |
| | P = .034 | P = .689 | P = .515 |

### Discussion

These results reveal significant translational motion in all three anatomical planes in native asymptomatic hips, supporting our first hypothesis. The observed associations between hip flexion and posterior translation, and between hip abduction and medial translation partially supported our second hypothesis. Finally, the observed associations between femoral anteversion, alpha angle, lateral center edge angle, acetabular inclination and neck shaft angle to range of femoral head translation in the anterior-posterior and superior-interior directions partially supported our third hypothesis.

While the research did not indicate an association between femoral rotations and femoral head translations in the superior-inferior direction, there were significant associations between flexion and posterior translation and between abduction and medial translation of the femoral head. These observations are in part consistent with prior reports of posterior, medial and inferior translations during FABER exam. While these agreements support potential links between hip rotations and translations, the fact that flexion and abduction rotations where only able to explain 4% (R²=0.04) of the variations seen in the posterior and medial translations, respectively, highlights the complexity of the interactions between hip rotations and translations. Further studies on larger cohorts are essential to better investigate these relationships with adequate statistical power to adjust for potential confounders.

Notably, the results indicated significant associations between some of the key morphological features of the hip, known to be involved in FAI and hip instability, and range of femoral head translations only in the anterior-posterior and superior-inferior directions. With regards to the anterior-posterior translation, we saw increased femoral anteversion correlating to increased translation. This is consistent with the effect of femoral anteversion on increased hip instability, in particular in the anterior direction. We also saw decreased anterior-posterior translation with increasing alpha angle, which could be due to limited space available for a more aspherical femoral head to translate inside the acetabulum. With regards to the superior-inferior translation, we saw decreased translations with increased femoral anteversion and neck-shaft angle. Decreased neck-shaft angle has been suggested to increase joint stability and shear stresses across the acetabulum. Such effects can result in higher friction between the femoral head and acetabulum, further minimizing the extent of femoral head translation. On the acetabular side, we found that increased acetabular inclination and decreased lateral center edge angle correlated with increased femoral head translation in the superior-inferior direction. Both increased acetabular inclination and decreased center edge angle have consistently shown to result in hip instability. A more vertical and shallower acetabulum will be less effective in limiting femoral heads translation, in particular in the superior-inferior direction. While, increased acetabular inclination and decreased lateral center edge angle are hallmarks of hip dysplasia and instability, the current findings, suggest that even in a normal asymptomatic hip, there is a certain degree of superior-inferior translation, which should be considered when evaluating hip instability and impingement.

There may be limitations to application of the current results. First, the data are collected on a small number of subjects with asymptomatic hips. Future large-scale cohorts on subjects with normal, FAI and unstable (e.g., dysplasia) hips are required to further investigate the normal and pathologic hip translations and their associations with hip rotations and anatomy. Second, the motions were all passive, which may result in different motion patterns than those seen during active movements. The choice of passive movement was made to replicate clinical exam conditions and to accommodate limited space and relatively long period of postural pause during MRI. The muscle activations could have resulted in additional joint stability, thus leading to different translations. Finally, all the motions were non-weightbearing, which may have influenced the observed translations.

In conclusion, the current findings support femoral head translation of up to 7 mm during physiologic ranges of rotations in normal asymptomatic hips. Data also highlight hip morphology and extent of hip rotation as significant contributors to femoral head translation. These translations can significantly influence femur's articulation inside the acetabulum, with direct impact on hip stability and impingement. Our results highlight the importance of further investigations to understand the normal and pathologic hip translation along with the need for tools to quantify and simulate hip translation during the clinical examination of the hip.

### Examples of Computer-Implemented Embodiments

Techniques operating according to the principles described herein may be implemented in any suitable manner. Included in the discussion above are a series of flow charts showing the steps and acts of various processes that analyze operation of joints. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one of ordinary skill in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of software. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

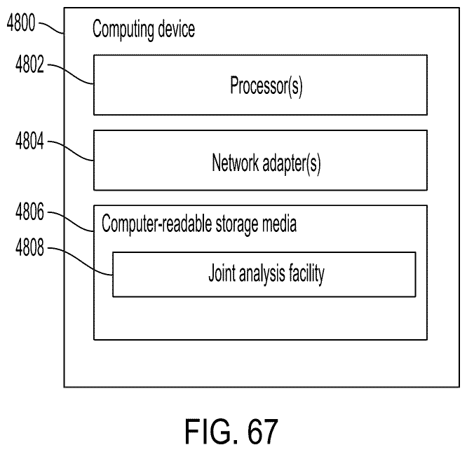

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 4806 of FIG. 67 described below (i.e., as a portion of a computing device 4800) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 1, or one or more computing devices (or one or more processors of one or more computing devices) may be programmed to execute the computer-executable instructions. A computing device or processor may be programmed to execute instructions when the instructions are stored in a manner accessible to the computing device/processor, such as in a local memory (e.g., an on-chip cache or instruction register, a computer-readable storage medium accessible via a bus, a computer-readable storage medium accessible via one or more networks and accessible by the device/processor, etc.). Functional facilities that comprise these computer-executable instructions may be integrated with and direct the operation of a single multi-purpose programmable digital computer apparatus, a coordinated system of two or more multi-purpose computer apparatuses sharing processing power and jointly carrying out the techniques described herein, a single computer apparatus or coordinated system of computer apparatuses (co-located or geographically distributed) dedicated to executing the techniques described herein, one or more Field-Programmable Gate Arrays (FPGAs) for carrying out the techniques described herein, or any other suitable system.

FIG. 67 illustrates one exemplary implementation of a computing device in the form of a computing device 4800 that may be used in a system implementing the techniques described herein, although others are possible. It should be appreciated that FIG. 67 is intended neither to be a depiction of necessary components for a computing device to operate in accordance with the principles described herein, nor a comprehensive depiction.

Computing device 4800 may comprise at least one processor 4802, a network adapter 4804, and computer-readable storage media 4806. Computing device 4800 may be, for example, a desktop or laptop personal computer, a personal digital assistant (PDA), a smart mobile phone, a server, a wireless access point or other networking element, or any other suitable computing device. Network adapter 4804 may be any suitable hardware and/or software to enable the computing device 4800 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 4806 may be adapted to store data to be processed and/or instructions to be executed by processor 4802. Processor 4802 enables processing of data and execution of instructions. The data and instructions may be stored on the computer-readable storage media 4806 and may, for example, enable communication between components of the computing device 4800.

The data and instructions stored on computer-readable storage media 4806 may comprise computer-executable instructions implementing techniques which operate according to the principles described herein. In the example of FIG. 67, computer-readable storage media 4806 stores computer-executable instructions implementing various facilities and storing various information as described above. Computer-readable storage media 4806 may store joint analysis facility 4808, which may implement any one or any combination of the techniques described above.

While not illustrated in FIG. 67, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure, and are intended to be within the spirit and scope of the principles described herein. Accordingly, the foregoing description and drawings are by way of example only.

What is claimed is:

1. A method comprising:
evaluating morphology and/or operation of a joint of an animal using a computer model of the joint, the joint comprising a first bone and a second bone that move relative to one another during use of the joint by the animal, the computer model of the joint comprising a first model of the first bone and a second model of the second bone, the evaluating the morphology and/or operation of the joint comprising:
determining the morphology of tissue of the joint, wherein determining the morphology comprises size/dimensions, orientations, relative positions, and/or alignments of one or more anatomical features of the first bone and/or the second bone;
determining a first amount of translation movement of the first bone to simulate;
determining a second amount of rotation movement of the first bone to simulate;
simulating movement of the first bone of the joint across a plurality of positions with respect to the second bone, the simulating of the first bone across the plurality of positions comprising simulating translation movement of the first bone in accordance with the first amount and simulating rotation movement of the first bone in accordance with the second amount; and
determining, based on the simulating of the movement of the first bone of the joint, information about operation of the joint; and
in response to determining that the morphology of the joint is improper and/or the information about operation of the joint indicates improper operation,
determining a recommendation on whether and/or how to treat the joint based on the information about the morphology and/or operation of the joint; and
outputting the recommendation on whether and/or how to treat the joint.

2. The method of claim 1, wherein determining information about operation of the joint comprises determining, based on the simulating, whether and/or where the first bone and second bone change position and/or impinge during operation of the joint.

3. The method of claim 2, wherein:
the method further comprises generating a heat map of morphology and/or impingement of the first bone and second bone in the joint; and
outputting the recommendation comprises outputting the heat map of morphology and/or impingement.

4. The method of claim 3, wherein:
the joint of the animal is a human hip joint; and
determining the recommendation based on the information about the morphology and/or operation of the joint comprises determining whether to recommend surgery or physical therapy to treat the improper operation of the joint.

5. The method of claim 4, further comprising:
obtaining information regarding locations of anatomical features of the first bone and the second bone within the human hip joint of a patient; and
generating the computer model of the human hip joint of the patient based on the locations of the anatomical features.

6. The method of claim 5, wherein:
determining information about operation of the joint comprises determining a translation amount for the human hip joint of the patient to satisfy at least one impingement and/or coverage criterion;
determining the recommendation on whether and/or how to treat the joint based on the information about the morphology and/or operation of the joint comprises:
determining whether the translation amount satisfies a physical therapy criterion; and
in response to determining that the translation amount satisfies the physical therapy criterion, determining that physical therapy should be recommended for the human hip joint.

7. The method of claim 6, wherein determining the recommendation on whether and/or how to treat the joint based on the information about the morphology and/or operation of the joint comprises identifying a portion of the first bone and/or the second bone to recommend adjusting in surgery.

8. The method of claim 1, wherein:
the joint of the animal is a human hip joint; and
determining information about operation of the joint comprises determining, based on the simulating, a measurement of femoral head coverage for the human hip joint.

9. The method of claim 1, further comprising:
prompting a user for the first amount of translation movement to simulate and the second amount of rotation movement to simulate.

10. The method of claim 1, further comprising:
analyzing medical imagery of the joint to generate the computer model of the joint, wherein analyzing the medical imagery comprises segmenting the medical imagery into the first bone and the second bone.

11. The method of claim 10, further comprising:
determining, from the medical imagery, locations of anatomical features of the first bone and the second bone within the human hip joint of a patient; and
generating the computer model of the human hip joint of the patient based on the locations of the anatomical features.

12. The method of claim 11, further comprising:
compiling, based on a plurality of medical images of the joint for different patients, models of normal and/or pathologic versions of the joint.

13. The method of claim 12, wherein the compiling models of normal and/or pathologic versions of the joint comprises compiling models for each of a plurality of demographic groups.

14. At least one computer-readable storage medium having encoded thereon executable instructions that, when executed by at least one processor, cause the at least one processor to carry out a method comprising:
evaluating morphology and/or operation of a joint of an animal using a computer model of the joint, the joint comprising a first bone and a second bone that move relative to one another during use of the joint by the animal, the computer model of the joint comprising a first model of the first bone and a second model of the second bone, the evaluating the morphology and/or operation of the joint comprising:
determining the morphology of tissue of the joint, wherein determining the morphology comprises size/dimensions, orientations, relative positions, and/or alignments of one or more anatomical features of the first bone and/or the second bone;
determining a first amount of translation movement of the first bone to simulate;
determining a second amount of rotation movement of the first bone to simulate;
simulating movement of the first bone of the joint across a plurality of positions with respect to the second bone, the simulating of the first bone across the plurality of positions comprising simulating translation movement of the first bone in accordance with the first amount and simulating rotation movement of the first bone in accordance with the second amount; and
determining, based on the simulating of the movement of the first bone of the joint, information about operation of the joint.

15. The at least one computer-readable storage medium of claim 14, wherein determining information about operation of the joint comprises determining, based on the simulating, whether and where the first bone and second bone change position and/or impinge during operation of the joint.

16. The at least one computer-readable storage medium of claim 14, wherein:
the joint of the animal is a human hip joint; and
the method further comprises determining, based on the information about the morphology and/or operation of the joint, whether to recommend surgery or physical therapy to treat the improper operation of the joint.

17. The at least one computer-readable storage medium of claim 16, wherein:
determining information about operation of the joint comprises determining a translation amount for the human hip joint of the patient to satisfy at least one impingement and/or coverage criterion;
determining whether to recommend surgery or physical therapy comprises:
determining whether the translation amount satisfies a physical therapy criterion; and
in response to determining that the translation amount satisfies the physical therapy criterion, determining that physical therapy should be recommended for the human hip joint.

18. An apparatus comprising:
at least one processor; and
at least one computer-readable storage medium having encoded thereon executable instructions that, when executed by the at least one processor, cause the at least one processor to carry out a method comprising:
evaluating morphology and/or operation of a joint of an animal using a computer model of the joint, the joint comprising a first bone and a second bone that move relative to one another during use of the joint by the animal, the computer model of the joint comprising a first model of the first bone and a second model of the second bone, the evaluating the morphology and/or operation of the joint comprising:
determining the morphology of tissues of the joint, wherein determining the morphology comprises size/dimensions, orientations, relative positions, and/or alignments of one or more anatomical features of the first bone and/or the second bone;
determining a first amount of translation movement of the first bone to simulate;
determining a second amount of rotation movement of the first bone to simulate;
simulating movement of the first bone of the joint across a plurality of positions with respect to the second bone, the simulating of the first bone across the plurality of positions comprising simulating translation movement of the first bone in accordance with the first amount and simulating rotation movement of the first bone in accordance with the second amount; and
determining, based on the simulating of the movement of the first bone of the joint, information about operation of the joint; and
in response to determining that the morphology of the joint is improper and/or the information operation of the joint indicates improper operation,
determining a recommendation on whether and/or how to treat the joint based on the information about the morphology and/or operation of the joint; and
outputting the recommendation on whether and/or how to treat the joint.

19. The apparatus of claim 18, wherein determining the recommendation based on the information about the morphology and/or operation of the joint comprises determining whether to recommend surgery or physical therapy to treat the improper operation of the joint.

20. The apparatus of claim 18, wherein the method further comprises:
determining, from medical imagery of the joint, locations of anatomical features of the first bone and the second bone within the joint; and
generating the computer model of the joint based on the locations of the anatomical features.
